(19) United States
(12) Patent Application Publication
(10) Pub. No.: US 2024/0367097 A1
(43) Pub. Date: Nov. 7, 2024

(54) AIR POLISHER PHOTONIC TARGET CHAMBER AND SYSTEM USING SAME

(71) Applicant: Violett Inc., Gig Harbor, WA (US)

(72) Inventor: Glen J. Bertini, Fox Island, WA (US)

(21) Appl. No.: 18/652,467

(22) Filed: May 1, 2024

Related U.S. Application Data
(60) Provisional application No. 63/463,520, filed on May 2, 2023.

(51) Int. Cl. B01D 53/32; A61L 9/18; B01D 46/00; B60H 3/06; F24F 8/10

(52) U.S. Cl. CPC B01D 53/32; A61L 9/18; B01D 46/00; A61L 2209/12; A61L 2209/14; B01D 2257/91; B01D 2258/06; B01D 2279/65; B60H 3/06; F24F 8/10

(57) ABSTRACT

An air polisher photonic target chamber for cleaning an airflow having entrained targets with photons, having an input aperture positioned to receive the airflow into the target chamber, an output aperture positioned to exhaust the airflow from the target chamber, with an internal target chamber airflow extending between the input and output apertures, a target chamber interior having a target chamber reflectance, at least one discrete unshadowed portion with an area having a homogenous material reflectance, and at least one discrete shadowed portion with an area having a homogenous reflectance and a homogenous shadow, where the areas together comprise the total interior area of the target chamber, and a chamber structure causing the chamber to have a relative improvement in the variance from ideal.

### BACKGROUND

### Field of the Invention

The present invention includes two classes of air polishers referred to interchangeably as photonic target chambers or target chambers and isentropic compression-expansion (ICE) machines. Both classes reduce the concentration of contaminants including pathogens in air. The two classes may be combined to enjoy certain synergies.

### Description of the Related Art

Definitions and acronyms which will be useful to an understanding of the present inventions are included alphabetically in the following three sections, namely Generic Definitions, ICE Definitions, and Photonic Definitions. Generic definitions are useful for both device classes, ICE definitions are useful to an understanding of an ICE machine, and photonic definitions are useful to an understanding of photonic target chambers.

Example devices, methods, and systems are described herein. It should be understood the words “example,” “exemplary,” “illustrative” and the like are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the figures, can be arranged, substituted, combined, separated, and designed in a wide variety of different configurations, all of which are explicitly contemplated herein.

### BRIEF DESCRIPTION OF THE SEVERAL VIEWS OF THE DRAWING(S)

Fig. APS-01A is a schematic drawing of an air processing system (APS) in accordance with an embodiment of the present disclosure.

Fig. APS-01B is a schematic drawing of one embodiment of a sensor module in an APS.

Fig. APS-02 is a schematic drawing of an exemplary APS and a partially enclosed space (PES).

Fig. APS-03 is a schematic drawing of three APS units connected in series.

Fig. APS-04 is a schematic drawing of three APS units connected in parallel.

Fig. PHO-01 is a graphical illustration of photonic efficiency as a function of photonic persistence at three different viral target concentrations.

Fig. PHO-02 is a log-log plot of unit performance as a function of unit reflectance for a 0.2-meter average photon path from 10% reflectance approaching 100%.

Fig. PHO-03 provides fractions of Lambertian (left y-axis) and specular (right y-axis) reflection as a function of AOI for reflectors that exhibit a combination of Lambertian and specular reflections.

Fig. PHO-04 is an overlay of two cross-sectional views of an exemplary three-target-chambered trapdoor device to produce cleaner air.

Fig. PHO-05A is a sectional elevation view of a single closed target chamber of an exemplary three-target-chambered trapdoor device to produce cleaner air.

Fig. PHO-05B is a sectional elevation view of a single open target chamber of an exemplary three-target-chambered trapdoor device to produce cleaner air.

Fig. PHO-05C is an exemplary design of stationary bulkhead designed to accommodate three target chambers.

Fig. PHO-06 is a sectional view of a positive 4-toothed sawtooth cylindroid target chamber polisher.

Fig. PHO-07 is a sectional view of a positive 4-toothed sawtooth cylindroid target chamber polisher illustrating a virtual shadow.

Fig. PHO-08 is a partial section view of a negative sawtooth cylindroid target chamber polisher.

Fig. PHO-09 is a sectional view of an exemplary embodiment of sawtooth cylindroid target chamber polisher including two partial cylindroids.

Fig. PHO-10A is a sectional view of a hexagonal prismatic photon vortex target chamber polisher.

Fig. PHO-10B is a three-dimensional wire-frame view of a hexagonal prismatic photon vortex target chamber polisher and an exemplary partial photon trajectory.

Fig. PHO-11A is a sectional view of PV target chamber polisher and a partial photon trajectory (i.e., about 3 reflections).

Fig. PHO-11B is a sectional view of PV target chamber polisher and an extended photon trajectory (i.e., about 61 reflections).

Fig. PHO-12 is a graphical view of values in Table 2 illustrating the trade-off between chord length and virtual shadow diameter.

Fig. PHO-13 is a PV target chamber polisher where a photon source and its associated optics do not produce a collimated beam.

Fig. PHO-14 is an illustration of Lambert's cosine law.

Fig. PHO-15 is a sectional view of an optical cavity or focal cone illuminated from a photon source located outside the focal cone.

Fig. PHO-16 is a sectional view of a right member of a pair of PRC mirrors together with a photon source and two non-imaging elements.

Fig. PHO-17A is a sectional view of a non-limiting example of photon rich cavity illustrating two pairs of initial edge rays from a photon source.

Fig. PHO-17B is a sectional view of a non-limiting example of photon rich cavity illustrating two pairs of edge rays after their first reflections.

Fig. PHO-17C is a sectional view of a non-limiting example of photon rich cavity illustrating the photon rich zones between two pairs of edge rays after repeated reflections.

Fig. PHO-18A is a sectional view of an exemplary concentrating baffle design for perpendicular flow of air from a duct across a conical PRC.

Fig. PHO-18B is a mixed sectional-isometric view of an exemplary vortex inducer design for perpendicular flow of air from a duct across a conical PRC.

Fig. PHO-18C is a sectional view of four virtual ducts designed for perpendicular flow of air from a duct across a conical PRC.

Fig. PHO-19A is a sectional view of an exemplary annular cylindroid photonotron.

Fig. PHO-19B is a sectional view of an exemplary annular cylindroid photonotron illustrating axial members to support an inner cylindroid.

Fig. PHO-20A is an axial cross-sectional view of a non-annular cylindroid photonotron.

Fig. PHO-20B is a longitudinal cross-sectional view of a non-annular cylindroid photonotron illustrating fluidic and photonic access.

Fig. PHO-21A is a simplified sectional view of a first toroidal photonotron.

Fig. PHO-21B is a perspective view of a first toroidal photonotron.

Fig. PHO-21C is a sectional view of a first toroidal photonotron bulkhead.

Fig. PHO-22A is a first cut-away perspective view of a second toroidal photonotron with an alternate means to introduce and withdraw air.

Fig. PHO-22B is a second cut-away perspective view of a second toroidal photonotron with an alternate means to introduce and withdraw air.

Fig. PHO-23 is a sectional view of a guardrail photon trap and a backstop photon trap.

Fig. PHO-24 is a summary of data from U.S. Pat. No. 10,800,672 illustrating UV transmittance and reflection as a function of thicknesses of a proprietary PTFE formulation.

Fig. PHO-25 is a collection of exemplary photon paths on a hybrid composite diffuse reflector.

Fig. ICE-01A is a perspective view of a 4-cylinder integrated cycle ICE machine with the crankshaft at a first angular position.

Fig. ICE-01B is a perspective view of a 4-cylinder integrated cycle ICE machine with the crankshaft at a second angular position, 180-degrees out of phase with the position in Fig. ICE-01A.

Fig. ICE-02 is a graph illustrating the thermodynamic relationships between static compression-expansion ratio and the temperature and pressure of air together with entrained T1K and T3K as a function of a static compression/expansion ratio from 1 to 30 for an ICE machine.

Fig. ICE-03 is a graph illustrating an exemplary temperature and pressure profile of an ICE machine covering 0.06 seconds.

Fig. ICE-04 is a flowchart illustrating cycles of an ICE machine.

Fig. ICE-05 is a graph illustrating the relationship between isentropic energy and isentropic temperatures in comparison to actual energy and temperatures.

Fig. ICE-06 is a graph illustrating an exemplary temperature and pressure profile of an ICE machine including an isochoric dwell covering 0.06 seconds.

Fig. ICE-07 is a simplified example of a split-cycle ICE machine.

Fig. ICE-08 illustrates pneumatic flow paths for an exemplary integrated-cycle ICE machine.

Fig. ICE-09 illustrates pneumatic flow paths for an exemplary split-cycle ICE machine.

Fig. ICE-10 is a graph that illustrates an approximate air flow as a function of time for single exemplary integrated cycle piston-cylinder.

Fig. ICE-11A is a graph that illustrates an approximate exhaust air flow as a function of time for a single exemplary integrated cycle piston-cylinder.

Fig. ICE-11B is a graph that illustrates an approximate exhaust air flow as a function of time four exemplary matched integrated cycle piston-cylinders.

Fig. ICE-11C is a graph that illustrates an approximate exhaust air flow as a function of time eight exemplary matched integrated cycle piston-cylinders.

Fig. ICE-12 illustrates a perforated core conduit muffler designed to modulate air flow and reduce noise.

Fig. ICE-13 illustrates a deformable elastomeric tube designed to modulate exhaust air flow.

Table 1 tabulates selected values (columns) for selected prismatic photon vortices (rows) with side length equal to L_{S}=1. “Adjacent non-acute faces” provides the number of adjacent faces that can be directly illuminated in the photon source cone without encountering an acute angle. “Vertex to diagonal” provides the length from the midpoint of a diagonal to the closest vertex. “Photon-free polygon radius” provides the radius of the polygon formed from the intersections of all vertex diagonals. The photon-free polygon is a photon-free zone if the cone width of the photon source is about equal to the acute vertex angle and the most parallel edge-ray is approximately parallel to a face adjacent to the photon source.

Table 2 tabulates selected values (columns) for selected whole number beam angles (rows) for circular cross sections of a PV chamber with a diameter, D. The beam angle is the angle in degrees of a ray introduced through an orifice from outside of a PV into a PV where the angle is measured from a tangent to the circular cross section at the inside surface of the orifice. The subtended angle is the angle of a vertex at the circle center of a triangle whose base is a chord, and the chord is a beam. Chord is the chord length expressed as a fraction of the circle diameter, D. Black hole diameter is expressed as a fraction of the PV chamber inner diameter, is less than or equal to the inside diameter of the PV chamber, shares the same center with a PV cross section, and is not illuminated by photons. Vertexes is the number of reflections experienced by a photon to reflect around the circle.

### DETAILED DESCRIPTION OF THE INVENTION

### Generic Definitions

### Aerosol

A suspension of tiny particles and/or droplets (i.e., solids, liquids, and solid-liquid agglomerates) in the air, such as dusts or mists.

### AMD

An acronym for air moving device, an AMD is a piece of equipment, at least one purpose of which is to move air from one place to another place. Non-limiting examples include fans, blowers, compressors, and ICE machines.

### APS

An acronym for an air processing stack. An APS is a combination of at least one air moving device (AMD), a polisher, optional sensors with associated firmware and software, an optional control module with associated firmware and software, an optional pre-polisher, and an optional post-polisher. The primary purpose of an APS is to reduce targets in an airflow. Targets are reduced within an APS between an exhaust air flow and a feed air flow.

### Clean Air

Pathogen-free, radionuclide-free, poison-free, pollutant-free, aerosol-free air. Dry clean air includes oxygen (about 21%), nitrogen (about 78%), argon (about 0.93%), carbon dioxide (about 419 ppm), and other trace gases (less than about 200 ppm combined). Other trace gases together with their respective typical tropospheric concentration include but are not limited to neon (18 ppm), helium (5 ppm), methane (1.7 ppm), oxides of nitrogen (1 ppm), hydrogen (0.6 ppm), and ozone (0.001 ppm). Clean air also includes about 0-5% water vapor. Substantial deviations from the clean air concentrations listed may result in adverse consequences. To illustrate, consider the adverse consequences of carbon dioxide levels above 419 ppm. The American Conference of Governmental Industrial Hygienists (ACGIH) recommends an 8-hour time-weighted average Threshold Limit Value (TLV) of 5,000 ppm and a ceiling exposure limit (not to be exceeded) of 30,000 ppm for a 10-minute period. A value of 40,000 ppm is considered immediately dangerous to life and health. All other substances not included in clean air are undesirable contaminants or targets.

### Cleaner Air

Cleaner air is a first air volume that is closer to clean air than a second air volume.

### Contact

Physical contact of at least one target with at least one protectee. Contact includes respiration, ingestion, skin contact, eye contact, or contact with any external or internal surface of a protectee.

### Designated Air

Air together with any entrained targets which is provided to a photonic target chamber, an ICE machine, or a hybrid ICE machine-target chamber.

### Exhaust Air

Cleaner air that exits an APS into a PES.

### Feed Air

Air that enters an APS intended to become cleaner.

### Protectee(s)

Human(s) or animal(s) to be protected from contact with targets.

### Emission Rate

A unit production of targets per unit time by protectee(s) within a PES or introduced to a PES by an air supply from outside of the PES.

### HEPA

A type of mechanical air filter. It is an acronym for “high efficiency particulate air [filter]” (as officially defined by the U.S. Dept. of Energy). This type of air filter can theoretically remove at least 99.97% of dust, pollen, mold, bacteria, and any airborne particles with a size of 0.3 microns (μm).

### PES

A PES is an at least partially enclosed space and may be a totally enclosed space. A PES may include but is not limited to a room, a plurality of rooms, a building, and a vehicle. A vehicle may include but is not limited to ground transports, air transports, water surface transports, underwater transports (e.g., submarines), and transports that operate at least part of the time in a partial vacuum or in space. A PES may have civilian, government, and military purposes. A PES is a space that is at least occasionally occupied by at least one protectee.

### Polished Air

Air together with any residual entrained targets and chemically altered targets which exhaust from a polisher.

### Polisher

A device that incorporates at least one of an IIO, an IIL, and a photonic target chamber.

### System

The combination of at least one APS, at least one PES, and at least one protectee. At least one protectee may be within a PES continuously, intermittently, or occasionally.

### System Lethality

A reduction in targets within a PES.

### System Performance

A comparative measure of system lethality within a system. The time integrated probability of a protectee being harmed by exposure to a target. System performance is measured as a change in a target concentration (AC) per unit time within a PES for a defined target emission rate. Concentration may be expressed as target quanta, target mass, or target volume per unit volume of air or log reductions of those values. A defined target emission rate may be a constant value or may be represented by dynamic values over time.

### Target(s)

Targets are things in air that are not in clean air and include T1K, T2K, T3K, T3Ka, T3Kb, T3Kc, T3Ka, and T4K defined infra. When the plural word “targets” is used in this document without one or more of the shorthand designations, T1K, T2K, T3K, T3Ka, T3Kb, T3Kc, T3Kd, and T4K, all kinds of targets are implicitly included. Where the singular word “target” is used in this document without modification it is referring to one specific target.

Targets may be introduced to the atmosphere by natural phenomena (Nonlimiting examples include volcanic eruption, secretions, evaporation, aerosolization, or exhalation by flora and fauna, radioactive decay of uranium to radon, and forest fires) or by human action (Nonlimiting examples include malevolent human action by terrorists and battlefield enemies, unpropitious human action such as industrial pollution and industrial accidents, and by inevitable human actions such as cooking and driving fossil-fueled vehicles).

### T1K

“Targets of a first kind” are biological substances including but not limited to pathogenic and non-pathogenic, flora and fauna, natural and engineered, living and non-living, complete and partial including reproductive portions such as spores. Examples include but are not limited to bacteria, bacterium spores, viruses, protozoa, helminths, helminth eggs, yeasts, yeast spores, mold, mold spores, fungus, fungus spores, animal dander, dust mites, prions, and other sources of proteins or other substances that might generate a biological response.

### T2K

“Targets of a second kind” are radioactive aerosols sometimes referred to as radioactive fallout. T3Ks fall into four categories designated as T3Ka, T3Kb, T3Kc, and T3Kd.

### T3K

“Targets of a third kind” are vapor phase or aerosolized chemicals not included in clean air excluding T4K.

### T3Ka

Category a T3Ks are oxidizable organic chemicals that upon complete oxidation yield essentially carbon dioxide and/or water. Non-limiting examples of T3Ka include but are not limited to methane in concentrations greater than that found in clean air, formaldehyde, and ethanol.

### T3Kb

Category b T3Ks are oxidizable organic chemicals that upon complete oxidation yield essentially carbon dioxide and/or water and/or nitrogen. Non-limiting examples of T3Kb include but are not limited to ammonia, isocyanic acid, hydrogen cyanide, and amino acids.

### T3Kc

Category c T3Ks are oxidizable chemicals that upon complete oxidation yield at least one of carbon dioxide and/or water and/or nitrogen, and at least one other oxidation product, where the at least one other oxidation product includes at least one atom other than oxygen, hydrogen, carbon and nitrogen. Examples of T3Kc include but are not limited to chlorosilane, hydrogen sulfide, thiols, HFC refrigerant R-32, octamethylcyclo-tetrasiloxane, and sarin (propan-2-yl methylphosphonofluoridate).

### T3Kd

Category d T3Ks are essentially non-oxidizable chemicals at the temperature, pressure, and dwell time of an ICE machine. Examples of T3Kd include but are not limited to ozone (above the concentration found in clean air) and NO_{X} (above the concentration found in clean air).

### T4K

“Targets of a fourth kind” include the singular naturally occurring radioactive noble gas, radon.

### VLP

Virus like particles are between 0.02 μm and 0.50 μm and contain nucleic acid.

### VOC(s)

VOCs are Volatile Organic Compounds. VOCs refers to organic compounds that will exist in a gas phase when at a temperature in the range of −10 to 50° C. Examples of VOCs include, but are not limited to: acetone, acrolein, acrylonitrile, allyl alcohol, allyl chloride, benzene, butene-1, chlorobenzene, 1-2 dichloroethane, ethane, ethanol, ethyl acrylate, ethylene, ethyl formate, ethyl mercaptan, formaldehyde, methane, methyl chloride, methyl ethyl ketone, propane, propylene, toluene, triethylamine, vinyl acetate, vinyl chloride, and other natural or human made gases which include at least one carbon atom, but excluding carbon oxides.

### ICE Definitions

### Adiabatic

A process in which heat does not enter or leave a system in a substantive way. A reversible, adiabatic process is always isentropic. No process is entirely adiabatic; adiabatic is to be understood to mean nearly adiabatic.

### Autoignition

Autoignition is a spontaneous initiation of an oxidation reaction in a mixture of air and T1K and/or oxidizable T3K. The autoignition temperature is a minimum temperature at which an oxidation or combustion process will occur in the absence of an ignition source and depends on pressure. Autoignition temperatures decrease with increasing pressure. The concentration of T1K and oxidizable T3K targets in a PES and the atmosphere is always extremely dilute, the fuel-air mixture is extremely lean, or ELF concentration. The concentration of oxidizable components is negligible and there is a stochiometric abundance of oxygen.

### Autoignition Delay Time

Autoignition delay time is an amount of time required for a T1K or oxidizable T3K at a temperature above the pressure dependent autoignition temperature to initiate oxidization. As a non-limiting illustration, methane has an autoignition temperature of about 580° C. at atmospheric pressure. If an extremely dilute mixture of methane and air is raised to 580° C., then the methane will eventually oxidize to produce H₂O and CO₂. However, if this same mixture is brought up to a higher temperature, for example 650° C., the ignition delay time might be 2 seconds. If the mixture is brought up to 750° C., the delay might be 0.1 seconds. Autoignition delay time is generally exponentially faster with higher temperatures. Autoignition delay times are also reduced by increasing pressure and are frequently inversely proportional to pressure. However, the exponent can range from −1 to −2. See Almohammadi et al, “Ignition and relight, and impact of alternative fuels” in Aviation Fuels, 2021.

### ELF

ELF is an acronym for extremely low fuel. An ELF concentration is a condition where the concentration of flammable T1K and oxidizable T3K in air is at least five-times lower than a lower flammability limit.

### IAC

An acronym for isentropic action condition.

### ICE Machine

ICE is an acronym for isentropic compression-expansion. An ICE machine isentropically induces oxidation (IIO) of T1K and oxidizable T3K and/or isentropically induces lysis (IIL) of T1K by a temporary increase in temperature and pressure (nearly adiabatic compression) from ambient, optionally followed with an isochoric dwell period, followed by a return to ambient conditions (nearly adiabatic expansion) where much of the energy required for the compression step is recovered in an expansion step.

### IIL

Isentropic Induced Lysis (IIL) is damage suffered by biological organisms exposed to adiabatic compression and expansion of air within an MVV and/or an isochoric oxidizer.

### IIO

Isentropic Induced Oxidation (IIO) occurs when oxidizable Targets (T1K and T3K) experience elevated temperature and pressure sufficient to induce at least some oxidation resulting from the adiabatic compression within an MVV. Subsequent expansion of air within an MVV returns the temperature and pressure close to initial values.

### IIO/IL APS Performance

A 1-log reduction occurs when 90% (1-nine) of a population of targets are destroyed in a single pass through an APS. 99% (2-nines) represents a 2-log reduction, 99.9% (3-nines) lethality represents a 3-log reduction, and N-nines represent an N-log reduction. APS performance may be different for each specific target.

### Integrated Cycle

An MVV where the following four cycles all occur within a single MVV: intake→compression→expansion→exhaust.

### Isentropic

A process where the change in entropy is about zero. No process is truly isentropic. In this document the word isentropic or its acronyms are to be understood to mean nearly isentropic or close to isentropic.

### Isentropic Action Condition (IAC)

A temperature-pressure condition in an isentropic compression-expansion cycle of air including at least one non-zero concentration of T1K and/or oxidizable T3K which demarcates a lower bound for decomposition (i.e., chemical reactions including partial and total oxidation) of T1K or oxidizable T3K, and/or denaturation of a T1K. An isentropic compression-expansion cycle optionally includes an isochoric dwell between compression and expansion portions of the cycle. Optional isochoric dwell optionally includes at least one catalyst. At temperatures-pressure conditions below an isentropic action condition, no decomposition or denaturation occurs. At temperatures-pressure conditions at or above an isentropic action condition, decomposition or denaturation may occur.

### Isochoric Dwell

A period of time delineated by a static compression ratio greater than or equal to 90% of the static compression ration maximum value. The period may be expressed with time units or as a fraction of the time taken for a MVV to complete four strokes.

### Isochoric Oxidizer

A constant volume reaction chamber that is essentially isobaric, essentially isothermic, essentially adiabatic, and essentially isentropic, in valved fluidic communication with a split-cycle compressor-MVV near its minimum volume and in valved fluidic communication with an expander-MVV near its minimum volume. The purpose of an isochoric oxidizer it to allow oxidation of targets (T1K and oxidizable T3K) and/or lysis of T1K to proceed outside of at least one pair of split-cycle MVVs.

### Lysis

Inactivation of organisms, viruses, nucleic acids, or proteins by changes in pressure that do not directly involve oxidation, but may proceed together with oxidation, are referred to as lysis.

### Maximum Volume

The point in a cycle of an MVV where the volume is maximized. For a piston engine this point is often referred to as bottom dead center or BDC.

### Minimum Volume

The point in a cycle of an MVV where the volume is minimized. For a piston engine this point is often referred to as top dead center or TDC.

### MVV(s)

A mechanically variable volume is a volume confined by walls, seals and valves such that mechanical action accommodates and/or urges said volume to increase or decrease. Non-limiting examples of MVV arrangements include piston engines and pistonless rotary engines of many designs including but not limited to Otto cycle, diesel, Atkinson cycle, Wankel, LiquidPiston (cam-guided rotary), Engineair, Hamilton Walker, Libralato rotary Atkinson cycle, quasiturbine, RKM, Sarich orbital, Trochilic, Wave disk, nutating disk, gerotor, IRIS (radial impulse), and turbines.

### NOX

“NO_{X}” refers to a group of oxides of nitrogen that include nitric oxide and nitrogen dioxide (NO and NO₂). There are at least three commonly acknowledged processes that form NO_{X}. “Thermal NO_{X}” is formed when oxygen and nitrogen present in air dissociate in the high temperature area of a combustion zone (at atmospheric pressure >1200° C.) and subsequently react to form oxides of nitrogen. See Lewander, M. (2011). Characterization and Control of Multi-Cylinder Partially Premixed Combustion”. Lund University, “Prompt NO_{X}” is formed in the proximity of a flame front as fuel fragments react with molecular nitrogen to form products such as HCN and atomic N, which are then oxidized to form NO_{X}. Because there are no flames in an IIO, this second mechanism is of no consequence in an ICE machine. “Fuel NO_{X}” is formed by combustion at very high temperatures of compounds containing nitrogen, e.g., amines including amino acids and cyano-ligands.

### Split-Cycle

An ICE machine that includes at least two MVVs, where at least a first MVV (compressor) executes intake and compression cycles and at least a second MVV (expander) executes expansion and exhaust cycles. MVV pairs are in valved fluid communication when a compressor

MVV and an expander MVV are both near their minimum volumes.

### ZMV

An acronym for zero minimum volume. A ZMV-MVV is a type of MVV used by a split-cycle ICE machine where the minimum volume of the MVV is zero or very close to zero.

### Photonic Definitions

### Active Shadow

A class of shadow created by a moving feature such as a trapdoor.

### AOI

Angle of incidence. A 0° AOI strikes a surface perpendicular to said surface. As AOI approaches 90° rays are sometimes described as glancing or grazing.

### APP

Average photon path (APP) is the average distance a photon translates through air between emission and striking a first surface and all subsequent translations between any two sequential reflections, until a photon is attenuated by reflection, absorption, or exits a target chamber through an orifice.

### Attenuation Shadow

A shadow created by a plurality of reflections along ray paths where each reflection absorbs and attenuates at least a portion of incident photons. An attenuation shadow is a type of passive shadow.

### BPT

An acronym for a backstop photon trap.

### Clock Shadow

A passive shadow created on the leeward side of a passive feature where the photonic flux is predominantly in a single clock direction.

### CRD

An acronym for cavity ring down.

### Deflection Shadow

The shadow created by the deflection of photons away from an orifice by convex surfaces proximate to said orifice.

### Diffuse Reflection

A reflection at a surface where photons' angles of reflection are predominantly not equal to the AOI. Some diffuse reflectors at some AOIs exhibit nearly Lambertian behavior.

### HCDR

An acronym for a hybrid composite diffuse reflector. An HCDR includes a primary diffuse reflective surface laminated to a secondary specular reflective surface.

### Lambertian Cosine Shadow

A shadow created by Lambert's cosine law where there is a relative scarcity of photons at angles of reflection approaching 90° (a.k.a. glancing or grazing angles) from a surface.

### Low Reflective Surface (LRS)

Any physical or virtual surface which has a first reflectivity, said first reflectivity is less than a second reflectivity. Said second reflectivity is the reflectivity of a predominant surface (i.e., greater than 50%) of a target chamber. Virtual surfaces include but are not limited to orifices. Physical surfaces include but are not limited to printed circuit boards, wires, sensors, light emitting diodes, and structural support elements.

### Orifice

A communication path between the inside and outside of a target chamber. Purposes for orifices include but are not limited to allowing photonic communication, allowing fluidic communication, and allowing wired communication. An orifice serves at least one purpose.

### Passive Shadow

A class of shadow created by a non-moving geometrical feature. There are five passive shadow types: clock shadows, deflection shadows, virtual shadows, attenuation shadows, and Lambertian cosine shadows.

### Photon Rich Cavity (PRC)

A zone of high photonic flux created from reflections between two or more mirrors.

### Photon Source

Any object that emits photons including but not limited to the sun, LEDs, lasers, incandescent bulbs, fluorescent bulbs, and mercury vapor lamps.

### Photonic Efficiency

The quotient of photon-target interactions and total photons entering a target chamber in like time periods.

### Photonic Lethality

Photonic lethality is the number of photon-strikes upon a T1K required to achieve a specific level of lethality and is represented by a one-dimensional array X₁, X₂, X₃, . . . . X_{N}.
Where the values of an array depend on wavelength(s) of incident photons.
A T1K is much larger than an incident photon and hence an impact location and direction of a photon determines, in part, its individual lethality and hence a cumulative lethality of a plurality of photons. Non-limiting examples include:
   a photon that interacts with a protein sheath of a virus may have lower lethality than the same photon striking a nucleic acid of the same virus, the latter being well known in the art to be susceptible to damage;
   the outcome of a photon-T1K interaction depends at least in part on a T1K temperature;
   a random quantum state of one or more electrons in a chemical bond which interacts with a photon impacts an outcome of an interaction.
It may take more than a single strike to be “lethal” to a target. “Lethal” is a relative term. Photons interact with specific bonds in complicated ways. Consequently, Photonic Lethality is defined statistically. A 1-log reduction occurs when 90% (1-nine) of a population of targets are destroyed by a cumulative X₁ photon-strikes. 99% (2-nines) represents a 2-log reduction and a cumulative X₂ photon-strikes, 99.9% (3-nines) represents a 3-log reduction and a cumulative X₃ photon-strikes, and N-nines represents an N-log reduction and X_{N} photon-strikes. Photonic lethality may be different for each specific T1K.

### Photonic Persistence

An estimate of a photon's survival in the absence of targets from its introduction into a target chamber to being absorbed upon impact with a surface or exiting target chamber through an orifice. Because the speed of photons in an atmosphere is essentially constant, photonic persistence can be measured interchangeably in units or time or distance.

### Photonic APS Performance

The product of photons, photonic persistence, and photonic lethality.

### Photonotron

A photon vortex that utilizes diffuse reflective surfaces.

### Photon Rich Zone (PRZ)

A portion of a volume within a target chamber which has a greater photon flux than the average photon flux of the target chamber.

### Photon Vortex

A reflective target chamber that urges photons in a predominant clock direction to create shadows for at least one orifice.

### Photons

In this document the word “photons” is used extensively to describe electromagnetic radiation in any range including but not limited to the UV range, the visible range, and the infrared range. Unless a wavelength is specified, an invention applies to all wavelengths of electromagnetic radiation and is a measure of how many photons are created per unit of time at one or more wavelengths or wavelength ranges.

### PIO

Photonic induced oxidation is the electromagnetic irradiation of a T1K or a oxidizable T3K within a target chamber to heat a T1K or a oxidizable T3K to indirectly induce oxidation of chemical bonds.

### PRC

Acronym for Photon Rich Cavity.

### PTFE

A polymer derived from polytetrafluoroethylene.

### PV

Acronym for Photon Vortex.

### Shadow (S)

A location within a target chamber where photonic flux is less than a photonic flux elsewhere within a target chamber. There are two shadow classes: passive shadows and active shadows. Shadows may be formed by either or both passive and active features. A shadow value, S, may range from 0 to 1 and may be expressed as a percentage. Where there is no shadow, S is 100%. Where there is a perfect shadow, S is 0%. For a partial shadow S lies between 100% and 0%. See also shadow equations 3 and 4.

### Specular Reflection

A reflection at a surface where photons' angles of reflection are predominantly equal to an AOI.

### Substantially Enclosed Volume

A volume encompassed by physical walls which are largely impervious to photons and air flow and static or dynamic virtual walls which delineate the interior and exterior of the substantially enclosed volume and that do allow the entry or exit of at least one of photons and air.

### Target Chamber

A substantially enclosed volume which retains and reflects at least some photons within the target chamber and allows air and entrained targets to pass through continuously or intermittently and where at least one device or method to improve chamber reflectivity is utilized from the list below:
1. Shadow (active or passive)
2. Photon rich cavity (PRC)
3. Guardrail photon trap
4. Backstop photon trap
5. Reflective material covering of Photon source ancillary mounting hardware
6. Hybrid composite diffuse reflector.

### Trapdoor(s)

A trapdoor in singular or plural form includes any moving member which may be actuated to temporarily cover at least one low reflective surface (LRS) to temporarily improve photonic persistence in a target chamber. Target chamber facing surfaces of a trapdoor are reflective surfaces.

### Virtual Shadow

A passive shadow created by judicious aiming of collimated or partially collimated photon source(s) such that subsequent reflections of a beam are not incident near or upon a virtually shadowed feature at least until the beam has been substantially attenuated.

### Virtual Wall

A soap bubble represents a gossamer physical analogy of a virtual wall. Virtual walls may be static or dynamic. A static virtual wall is the surface with the minimum area which virtually fills non-moving orifices. A dynamic virtual wall is a time dependent surface virtually filling the minimum area of a moving orifice at each location of a moving active shadow component. The perimeter of a static virtual wall is defined by the interior-most edge of its physical wall(s). The perimeter of a dynamic virtual wall is defined by the interior-most edge of its physical wall(s) at the location where the distance between at least two surfaces is a minimum.

### Methods (Filtration, Sorption, Dilution, Photocatalytic, Ozone, Irradiation, PIO, IIO, IIL)

The present invention focuses on four of nine methods to neutralize or mitigate airborne targets. The nine methods can be used independently, or a plurality of methods may be integrated to provide overlapping mechanisms to achieve desired system performance.
1. Filtration: Trap non-gas-phase targets (i.e., T2K) with filtration. Utilize porous media to trap solid and liquid aerosols. E.g., HEPA.
2. Sorption: Trap gas-phase targets (i.e., T3K) with sorption including adsorption and absorption. E.g., activated carbon, zeolites.
3. Dilution: Where a concentration of at least one target in air is greater within a first PES volume, compared to a second nearby volume, including but not limited to the Earth's atmosphere, air from the second volume can be supplied to the first volume while air from the first volume is vented or purged away from protectees. Dilution is the only way to reduce T4K.
4. Photocatalysis: A photon activated chemical reaction involving a catalyst to induce oxidation of oxidizable targets.
5. Ozonation: A creation of ozone to induce oxidization of oxidizable targets.
6. Irradiation: Irradiate targets within a target chamber with high energy photons to directly break chemical bonds. Once broken bonds may react with oxygen and become oxidized in a second chemical step.
   a. UV exposure or other wavelengths with germicidal properties.
   b. Gamma rays or other ionizing radiation.
7. PIO: Photonic induced oxidation is the electromagnetic irradiation of a target within a target chamber to heat a T1K or a oxidizable T3K to indirectly induce oxidation of chemical bonds.
8. IIO: Isentropically induced oxidation of a T1K or a oxidizable T3K from adiabatic compression and expansion of air within at least one mechanically variable volume (MVV) and/or an isochoric oxidizer.
9. IIL: Isentropically induced lysis of a T1K from adiabatic compression and expansion of air within at least one mechanically variable volume (MVV) and/or an isochoric oxidizer.

A target chamber in 6 and 7 above is a substantially enclosed volume to retain and reflect photons within a chamber volume and allows air together with entrained targets to pass through continuously or intermittently. An MVV and/or an isochoric oxidizer in 8 and 9 above includes a volume where air and entrained targets are isentropically compressed to momentarily increase pressure and temperature within a volume, optionally maintain an increased pressure and temperature for an isochoric dwell period and are subsequently isentropically expanded. An MVV and an isochoric oxidizer may also be a target chamber.

### HEPA

This document does not introduce new filtration technologies. HEPA filters and the like are extensively disclosed in the prior art. Aerosol filtration enjoys the following synergistic advantages with inventions in this disclosure.
1. Effectively reduce T2K.
2. Effectively reduce large T1K such as most bacteria.
3. Reduce aerosols within an APS which might interfere with downstream processing including:
   a. Attenuate photons within an APS air stream.
   b. Contaminate reflective surfaces and attenuate photons striking said contaminants.
   c. Contaminate mechanical seals, increasing friction and mechanical wear.

### Dilution

Dilution has been practiced since the first human cave dweller stepped out of a PES to get a breath of fresh air. Dilution of stale PES air with fresh air which requires venting a like quantity of stale PES air enjoys the following synergistic advantages with inventions in this disclosure.
1. Effectively reduce targets and/or carbon dioxide within a PES when a concentration of at least one target and/or carbon dioxide is less in a fresh air supply than a concentration of an at least one target and/or carbon dioxide within a PES. This condition is common when targets and/or carbon dioxide are being introduced within a PES. Purging air from a PES and diluting the air in the PES with fresh air is a practical method to reduce T4K.
2. Improve livability (i.e., cleaner air) within a PES when a surrounding atmosphere is contaminated with targets, applying at least one of Methods 1-2, and 4-9 (listed above) to a fresh air supply to a PES.

### Photocatalysis & Ozone

The present invention does not include improved photonically enhanced oxidizing technologies that create ozone or free radicals (e.g., hydroxy free radical) including photocatalytic processes. However, such processes that are known in the art may be incorporated in addition to any of the embodiments of the present invention introduced to enjoy advantages and synergies.

### Integration

Fig. APS-01A is a pneumatic flow chart of some elements of system 10100. System 10100 includes air processing stack 10120, or APS 10120, and gas sources 10110 which include sources of air and at least one protectee 10140 who is at least occasionally within PES 10111. Protectee 10140 is exchanging air together with things not found in clean air with PES 10111 air 10115. It is to be understood that the exchange is mutual. That is, protectee 10140 is inhaling air 10115 which includes things not found in clean air and protectee is exhaling air together with things not found in clean air. Generally, exhaled air is not as clean as inhaled air. PES 10111 may be in fluidic communication with air sources not included in airspace 10115 of PES 10111 including but not limited to atmosphere 10112 (i.e., outdoor air), manufactured air 10113 (e.g., electrolysis; cryogenic separation), and stored air 10114 (e.g., pressurized gas; cryogenic liquid). Each air source excluding airspace 10115 may include a restriction on flow rate. Restriction 10116 limits purging of air from PES 10111 to atmosphere 10112. Restriction 10117 limits a flow of air from atmosphere 10112 to PES 10111. Restriction 10118 limits a supply of manufactured air 10113 to PES 10111. Restriction 10119 limits a supply of stored air 10114 to PES 10111. For typical constant atmospheric pressure circumstances a flow of purging air through restriction 10116 is about equal to a sum of flows through restrictions 10117, 10118 and 10119. When it is desired to alter a pressure within PES 10111, the various flow restrictions may be altered to decrease or increase pressure in PES 10111. Non-limiting examples where a pressure change might be desirable include air locks, hyperbaric chambers, clean rooms, pressurized aircraft, spacecraft, and submersibles.

Air purge through restriction 10116 to atmosphere 10112 is a practical method to reduce a T4K concentration, a carbon dioxide concentration, a nitrogen concentration, a water vapor concentration, or any component in PES volume 10115 which has a concentration in PES volume 10115 greater than its concentration in atmosphere 10112.

Where atmosphere 10112 is itself contaminated it may be desirable to process atmosphere 10112 through APS 10120 before it is introduced to PES 10111. Sources of atmospheric contamination include but are not limited to volcanic eruptions, forest fires, industrial accidents (e.g., chemical, biological, nuclear), state sponsored or terrorist use of biological agents, chemical agents, and/or nuclear weapons. Whenever a protectee might be harmed by atmosphere 10112, APS 10120 may be used to improve the quality of air delivered from atmosphere 10112 to PES 10111. When atmospheric contamination is an issue restriction 10117 may be closed or limited and restriction 10199 is opened.

Restrictions 10116, 10117, 10118, 10119, and 10199 may be controlled or uncontrolled. Non-limiting examples of uncontrolled restrictions include open or closed doorways, open or closed windows, drafty building interfaces, orifices, inlet ducts, inter-PES ducts, and outlet/exhaust ducts. Non-limiting examples of controlled restrictions include valves and air moving devices or AMDs which may or may not be part of an HVAC system. AMDs may be controlled either manually or by non-manual means. Non-manual means include but are not limited to electronic control module 10180 which may be in wired or wireless communication with one or more controllable elements including AMD 10121, AMD 10123, AMD 10125, AMD 10127, restriction 10116, restriction 10117, restriction 10118, restriction 10119, restriction 10199, pre-polisher 10122, polisher 10124, and post-polisher 10126. Electronic control module 10180 may include a method for users to interface with control module 10180 to observe an operation status, establish control setpoints, and the like. Electronic control module 10180 may be in wired or wireless communication with sensor module 10198.

System 10100 includes air processing stack 10120, or APS 10120. APS 10120 includes at least one AMD. If at least one polisher 10124 is an ICE machine, polisher 10124 is an AMD and another AMD may not be required. If polisher 10124 is not an ICE machine, at least one of AMD 10121, AMD 10123, AMD 10125, and AMD 10127 is required. For most applications a single AMD provides a required air flow and second, third, and forth AMDs may be omitted. One or more AMDs may be stacked in any of the positions indicated within APS 10120. Each AMD 10121, 10123, 10125 and 10127 may be a single AMD or a plurality of AMDs connected in series and/or parallel. The choice of where to place an AMD and how many are required depends on pressure drop considerations, required air flow, AMD type, noise considerations, and design convenience. These choices and considerations are well known in the art.

Elements of APS 10120 are interconnected with essentially airtight flow paths. Flow path 10130 returns exhaust air from APS 10120 to PES 10111. Flow path 10131 delivers feed air from PES 10111 to AMD 10121 if present. If AMD 10121 is absent, flow path 10131 delivers air to flow path 10132. Flow path 10132 delivers air to optional at least one pre-polisher 10122. If pre-polisher 10122 is absent, flow path 10132 delivers air to flow path 10133. Flow path 10133 delivers air to AMD 10123 if present. If AMD 10123 is absent, flow path 10133 delivers air to flow path 10134. Flow path 10134 delivers air to at least one polisher 10124. Flow path 10135 delivers air to AMD 10125 if present. If AMD 10125 is absent, flow path 10135 delivers air to flow path 10136. Flow path 10136 delivers air to optional at least one post-polisher 10126. If post-polisher 10126 is absent, flow path 10136 delivers air to flow path 10137. Flow path 10137 delivers air to AMD 10127 if present. If AMD 10127 is absent, flow path 10137 delivers air to flow path 10130 and returns cleaner air to PES 10111.

At least one flow path 10130, 10131, 10132, 10133, 10134, 10135, 10136, and 10137 may include an elastomeric element to dampen flow variations and resulting sounds. An exemplary flow path to dampen flow variations and sound may be in the form of an elastomeric conduit. Said conduit expands when flow and pressure therein momentarily surge and contracts when flow and pressure momentarily decrease.

A first purpose of optional pre-polisher 10122 is to remove T2K and large particulates including some T1K that might interfere with polisher 10124 operation or longevity. A second purpose is to remove T3K that is not addressed by polisher 10124. An optional third purpose is to remove excess carbon dioxide. If T2K, T3K, large particulates, or excess carbon dioxide need not be addressed by system 10100, pre-polisher 10122 may be omitted. Where pre-polisher 10122 is included on APS 10120, pre-polisher 10122 may include any combination including pluralities of each of at least one device well known in the art including but not limited to: a course particulate prefilter, a chemically active filter, a photocatalytic surface, a free-radical generator, a HEPA filter, an electrostatic precipitator, ionizing radiation, an adsorption media, an absorption media, and a carbon dioxide scrubber.

Polisher 10124 is an ICE machine, a target chamber utilizing a method of the present invention, an ICE machine with an integrated target chamber, or an ICE machine and a separate target chamber utilizing at least one method of the present invention. Polisher 10124 is fed designated air via flow path 10134 and produces polished air and delivers it to flow path 10135. Polished air in flow path 10135 has lower concentrations of targeted targets than designated air in flow path 10134.

A first purpose of optional post-polisher 10126 is to remove any T1K, T2K, or T3K either not removed by the preceding elements of APS 10120 and/or any T1K, T2K or T3K introduced by the preceding elements of APS 10120. A first example of how preceding APS elements might introduce T3K follows: when T1K undergoes PIO, IIL, IIO, and/or irradiation T3K may be produced as a by-product. A second example of how preceding APS elements might introduce T3K follows: when polisher 10124 is an ICE machine, lubricant volatile organic components (i.e., VOCs or T3K) may evaporate and be swept along with polished air in flow path 10135.

An optional second purpose of optional post-polisher 10126 is to remove excess carbon dioxide. If T1K, T2K, T3K and carbon dioxide are at acceptably low levels in polished air in flow path 10135, post-polisher 10126 may be omitted. Where post-polisher 10126 is included on APS 10120, post-polisher 10122 may include any combination of at least one device well known in the art including but not limited to: a course particulate prefilter, a chemically active filter, a photocatalytic surface, a free-radical generator, a HEPA filter, an electrostatic precipitator, ionizing radiation, an adsorption media, an absorption media, and a carbon dioxide scrubber.

An optional third purpose of optional post-polisher 10126 is to dampen flow variations and resulting sounds from upstream components in APS 10120 including AMD 10121 (if present), pre-polisher 10122 (if present), AMD 10123 (if present), and polisher 10124.

APS 10120 may include optional sensor module 10198. If optional sensor module 10198 is present it is in wired or wireless communication with control module 10180. A single sample line is required for sensor module 10198. In Fig. APS-01A two sample lines are illustrated to avoid cluttering the description, but samples lines may be installed in any or all of the following locations when present: flow path 10130, flow path 10131, flow path 10132, flow path 10133, flow path 10134, flow path 10135, flow path 10136, flow path 10137, and flow path 10138. Sample return line 10171 returns air which has been processed by sensor module 10198 to near beginning of APS 10120 at flow path 10131 to be sure that it is processed by entire APS 10120 before ultimately being returned to PES 10115.

Fig. APS-01B provides detail of a preferred embodiment of sensor module 10198. Sensor module 10198 includes at least one sampling valve. In this example a first sample valve 10175 is periodically actuated by control module 10180 from its normally closed position to allow air to pass from flow path 10130 through sample tube 10170 to sample header 10177. All other sample valves remain in a closed position while first sample valve 10175 is open. A second sample valve 10176 is periodically actuated by control module 10180 from its normally closed position to allow air to pass from flow path 10132 through sample tube 10172 to sample header 10177. All other sample valves remain in a closed position while second sample valve 10176 is open.

Any number of sample tubes and sample valves may be added to provide samples of air throughout APS 10120. Sample header 10177 provides air flow from a singularly open valve and from a single location in APS 10120 to sensor package 10178. Each sample valve remains open long enough to purge air from sample tubing, sample header 10177, and sensor package 10178 and for the sensor to record the data.

Optional sensor package 10178 may include at least one sensor including but not limited to carbon monoxide concentration, NO_{X} concentration, temperature, pressure, humidity, carbon dioxide concentration, particulate count and size distribution, VOC concentration, biological assays, oxygen concentration, and any concentration of any target.

Optional AMD 10179 is only necessary if at least one sample point (e.g., flow path 10130 or flow path 10132) has a dynamic pressure less than or equal to a dynamic pressure at a sample return point (e.g., flow path 10131). Optional AMD 10179 is illustrated downstream of sensor package 10178, but the two components may be reversed without departing from the present invention.

While it is within the scope of the present invention to have a plurality of sensor modules, one for each sample point, a single sensor module that samples toggling air streams from a plurality of locations as illustrated in Figs. APS-01A and APS-01B is particularly advantageous as control module 10180 can calculate at least one differential concentration for each analyte of interest. Differential concentration is largely insensitive to sensor drift.

Elements of APS 10120 may be integrated into a single device or may be dispersed into sub-units with air ducts and controls linking disparate subunits. Fig. APS-02 illustrates three exemplary configurations. In configuration 10210 APS 10211 is entirely within PES 10215 and there is no gas source except PES 10215. APS 10211 is in intimate fluid communication with PES 10215. Intimate fluid communication occurs when an APS inlet or outlet (i.e., flow path 10130 and flow path 10131 of Fig. APS-01A are open to a PES).

In configuration 10220, APS 10221 is outside PES 10225 and is in fluid communication with PES 10225 and gas source 10224 via flow path 10223. Air return path 10222 returns cleaner air to PES 10225.

In configuration 10230, APS 10231 is partially inside and partially outside PES 10225. APS 10231 is in intimate fluid communication with PES 10235. Optional gas source 10234 is in fluid communication with APS 10231 via flow path 10233.

As illustrated in Fig. APS-03, APS may be combined in series. For a given flow rate multiple APSs may reduce target concentrations compared to a lessor number of APSs. While three APSs, 10321, 10322, and 10323, are illustrated, a single APS or a plurality of APS are included in the scope of the present invention. In Fig. APS-03 air is withdrawn from gas sources 10310, analogous to gas sources 10110 in Fig. APS-01A and fed via flow path 10331 to APS 10321. After processing by APS 10321, air is fed via flow path 10332 to APS 10322. After processing by APS 10322, air is fed via flow path 10333 to APS 10323. After processing by APS 10323, air is fed via flow path 10330 to PES 10315.

As illustrated in Fig. APS-04, APS may be combined in parallel. Multiple APSs may allow higher flow rates with the same target concentration reductions compared to a single APS. While three APSs, 10421, 10422, and 10423, are illustrated, a single APS or a plurality of APSs are included in the scope of the present invention. In Fig. APS-04 air is withdrawn from gas sources 10410 and fed via flow path 10431 to APS 10421, APS 10422 and APS 10423. After parallel processing, air is conveyed via flow path 10430 to PES 10415.

### System Performance

An APS is part of a system. In its simplest incarnation a single APS is placed in a PES and that PES at least occasionally includes at least one protectee. Those three elements: An APS, a PES, and at least one protectee comprise a system. The primary purpose of an APS is to reduce the quantity of airborne targets in a PES. A reduction in airborne targets is measured as a system lethality. An increase in system lethality requires that more targets be neutralized per unit time in a PES.

Without the protectee element of that 3-part system, system performance would be meaningless. As will be disclosed an APS can and should address all three elements of system performance. However, the bulk of the present invention focuses on APS performance.

### ICE Machine APS Performance

Within the scope limiting envelope of IIO/IIP (Methods 8 and 9, listed above) and collectively isentropic compression-expansion (ICE), APS performance is defined statistically. A 1-log reduction occurs when 90% (1-nine) of a population of targets are destroyed in a single pass through an APS. 99% (2-nines) represents a 2-log reduction, 99.9% (3-nines) lethality represents a 3-log reduction, and N-nines represent an N-log reduction. APS performance may be different for each specific target.

### Photonic APS Performance

Within the scope limiting envelope of photon bombardment (Methods 6 and 7, listed above), APS performance is defined by Equation 1.

$$ASP Performance = Photons X Photonic Efficiency X Photonic Lethality \qquad ( 1 )$$

Photonic persistence is directly proportional to photonic efficiency. Because so few photons strike targets before they are lost by reflective attenuation, the life of an average photon in the absence of targets is approximately equal to the life of a photon in the presence of targets.

Photonic persistence is the average lifetime of a photon within a target chamber. Photonic persistence can be expressed interchangeably in time (e.g., nanoseconds) or photonic range (e.g., meters).

Fig. PHO-01 illustrates photonic efficiency displayed on Y-axis 0120 as a function of photonic persistence on X-axis 0110 for three exemplary concentrations chosen to delineate a high concentration case 0150, a moderate concentration case 0140, a low concentration case 0130. The phrases “high concentration” “moderate concentration” and “low concentration” as used here distinguish each from the others. Compared to a lower flammability limit, all three of these values are ELF concentrations. The values used were measured by Prussin et al. (Prussin A J 2nd, Garcia E B, Marr L C. Total Virus and Bacteria Concentrations in Indoor and Outdoor Air. Environ Sci Technol Lett. 2015; 2 (4): 84-88. doi: 10.1021/acs.estlett.5b00050. PMID: 26225354; PMCID: PMC4515362. Prussin's Table 1 provides VLP (virus like particle) measurements at nine locations. Fig. PHO-01 uses the mean value (μ=1.2×10⁶ particles/m³) plus one standard deviation (σ=0.7×10⁶ particles/m³) of the highest concentration location, outdoors (1.9×10⁶ particles/m³), as the high concentration case 0150, the mean value of a moderate concentration location, a day care center (μ=4.5×10⁵ particles/m³), as the moderate concentration case 0140, and the mean value (μ=2.9×10⁵ particles/m³) less one standard deviation (σ=2.3×10⁵ particles/m³) from the lowest concentration location, a health center (6.0×10⁴ particles/m³), as the low concentration case, 0130. The range from lowest to highest concentration is about 31.7-times. Prussin et al believe the high outdoor concentration of VLPs is driven by bacteriophages which are generally benign to protectees.

The diameter of a typical coronavirus or influenza virus is about 100 nm and a typical cross-section profile of about 7,854 nm². The product of an individual virus cross section profile by a number of viruses in a spherical target chamber sphere with a 30 cm diameter provides the aggregate virus cross section and is compared with a cross-section of a sphere to determine the single-pass probability of a collision. Reflectivity within an APS target chamber determines the number of passes an average photon will persist in a target chamber. This value is multiplied by the single-pass probability to estimate the probability of a single photon-T1K collision.

Vertical line segment 0160 delineates an operation of a typical contemporary high performance UV air purifier with a photonic persistence of about 1 meter. Fig. PHO-01 demonstrates that for photonic persistence between 1 and 10 meters, photonic efficiency lies between about one-in-a-million (horizontal gridline 0170) and about one-in-a-billion (X-axis 0110). The remainder of the approximately 99.9999% to 99.9999999% of photons emitted into a target chamber provides no value.

### Reflectivity is an APS Issue

Fluid inlets and outlets, LEDs or lamps, sensors and other inherently low-reflective (including non-reflective) hardware should be minimized within a photon stream or target chamber. Where diffuse reflectors are utilized in conventional designs virtually all low-reflective hardware is in an omnidirectional photon deluge. With a specular reflective surface, it is possible to direct a photon stream over a course of a plurality of reflections. Some diffuse reflectors demonstrate substantial specular reflection as an angle of incidence approaches 90°. Photons scatter into diffuse paths from interactions with fluid molecules, interactions with particles suspended in the fluid, imperfections on a surface of a reflector, edges which diffract photons, and contaminants on a reflector.

A APS's target chamber reflectivity is the product of a normalized reflectivity (i.e., normalized to a sphere of the same volume as a non-spherical target chamber) of a reflective material lining (e.g., sintered PTFE and expanded PTFE have reflectivity of about 95%) and the difference between 1 and a fraction of the non-shadowed low-reflective surfaces. For example, if 10% of the inner surface of a spherical target chamber is non-reflective, the difference would be 90%. Thus, for the example provided, the system reflectivity would be the product of about 95% and about 90%, or about 85.5% (i.e., product of 90% reflective area and 95% reflectance plus product of 10% non-reflective area and 0% reflectance).

Even a perfectly reflective surface would not yield a perfectly reflective target chamber because of a requirement for low-reflective surfaces. Minimizing a surface area of low-reflective or non-reflective surfaces and/or shielding them in shadows maximizes target chamber reflectivity.

Prior art is replete with words like amplify and multiply to explain the benefit of higher reflectance. Such wording is misleading because there is no amplification, and the only multiplication is a ratio of an improvement's performance to some lower performing strawman. In this disclosure improvements in APS target chamber performance are achieved by mitigating photon attenuation with novel means. An unbiased measure of performance is photonic persistence.

Shadows can be created with one or more of the following three methods. The first two methods depend on geometrical shapes and the third relies upon photonic attenuation.
1. Passive geometrical features-see four sections entitled Clock Shadow, Defection Shadow, Virtual Shadow, and Lambertian Cosine Shadow.
2. Active geometrical features-active shadows-see section entitled Active Shadow.
3. Photon attenuation-see section entitled Attenuation Shadow.

### APS Target Chamber Reflectivity: Key to APS Target Chamber Performance

T1K in HEPA filtered air are a dilute suspension. For any one photon emitted, the probability that said photon strikes a target before it is absorbed by a surface within a target chamber is between about one in a million and one in a billion as illustrated in PHO-01. There are 5 ways to increase the probability of such collisions.
1. Increase the number of photons emitted. This is a brute force approach and suffers from the fact that photonic sources are themselves low reflectivity surfaces that reduce photonic persistence in a target chamber.
2. Increase path length before photons strike a surface. For example, a target chamber may be increased in size, the geometry can be morphed to a more spherical shape, or angles of incidence can be altered to encourage longer reflective paths.
3. Increase the number of times a photon is reflected from internal surfaces before being attenuated.
   a. Make internal surfaces more reflective,
   b. Choose angles of incidence to maximize reflectivity on a reflective surface,
   c. reduce a quantity of low-reflective surfaces not in shadow, and/or
   d. improve shadowing on low-reflective surfaces. An improved shadow enjoys a lower photonic flux within a shadowed volume compared to a second baseline shadow.
4. Increase concentration of targets. For gaseous fluids, this can be accomplished by:
   a. increasing gas pressure (i.e., doubling pressure doubles target concentration and doubles system performance) and/or
   b. lowering temperature. Lowering temperature requires a great deal of energy and is less preferred. To achieve the same doubling of performance as a doubling of pressure requires a halving of absolute temperature. Thus 25° C. air (298° K) would have to be cooled to −124° C. (149° K), and/or
   c. utilizing ultra-centrifugal forces to concentrate aerosols from gaseous components. This high energy approach is less preferred.
5. Increase residence time of targets within a target chamber.
   a. Make a target chamber larger (Refer also 2 above), and/or
   b. Reduce a flow rate, and/or
   c. Increase a pressure (See 4a above), and/or
   d. Decrease a temperature (See 4b above), and/or
   e. Concentrate targets with ultra-centrifugation, separate an air flow into a zero to low target stream and a concentrated target stream. The zero to low target stream bypasses a target chamber and a concentrated stream is processed in a target chamber or exhausted away from protectees.

APS target chamber performance with a 0.2-meter average photon path (APP) is impacted by an APS's target chamber reflectance as shown in Fig. PHO-02, where the APS target chamber reflectance from 0% to 99.99% is provided on X-axis 0210 and APS target chamber performance is plotted as curve 0230 against Y-axis 0220.

### Flight Path Length & Photonic Range

For any APS target chamber reflectivity less than 100%, a mean distance between reflections makes a substantial contribution to photonic persistence. With each reflection the probability a photon will be lost by absorbance or transmittance is a difference between unity and target chamber reflectivity. All else being equal, a doubling of an average photon path doubles photonic range which doubles a probability a photon will strike a target, rather than be absorbed on reflection or exit an orifice. Flight path cannot be considered without also considering the angle of incidence (AOI). For diffuse reflection a relationship between an angle of incidence and a distribution of angles of reflection is much more complex. All reflecting surfaces have a mix of specular and diffuse behavior. For optical instruments like a telescope mirror, great care and expense is made to minimize diffuse behavior. Despite all effort, reaching 100% specular reflection is a quixotic undertaking. A single dust particle, a single nano-scale imperfection in mirror smoothness, a single human fingerprint, a single quantum fluctuation introduces a degree of diffuse behavior. Likewise, even a superb Lambertian surface reflects photons with a specular component. Throughout this document, for the sake of brevity, and unless defined explicitly otherwise:
1. When referring to diffuse or Lambertian reflective surfaces it means a surface that exhibits predominantly diffuse reflection especially at low AOI. Most diffuse reflective surfaces exhibit more and more specular reflection as the AOI increases.
2. When referring to specular reflective surface it shall be understood to be referring to predominantly specular reflective surfaces.

### Angle of Incidence (Specular Reflection)

For specular reflection the angle of incidence impacts reflectivity. For some surface types, very large angles (glancing a surface) reflectivity is higher than for steeper angles (i.e., those with trajectories closer to normal to the surface). Reflectivity is often lowest near 0°. For systems designed to maximize specular reflection, angle of incidence and flight path length must be considered together. Specular reflection may include reflections from metallic surfaces such as aluminum, silver, and gold. The metal surface may be coated to mitigate oxidation. Specular reflection may also include distributed Bragg reflectors (DBR). A Bragg mirror (DBR or a dielectric mirror) is a mirror structure which consists of an alternating sequence of layers of two different dielectric optical materials. A frequently used design is that of a quarter-wave mirror, where each optical layer thickness corresponds to about one quarter of a wavelength for which a mirror is designed. The latter condition holds for normal incidence; if the mirror is designed for larger angles of incidence, thicker layers may be needed to maximize reflectance.

### Angle of Incidence (Diffuse Reflection)

Polymeric diffuse reflectors (e.g., sintered PTFE and expanded PTFE) involve at least partially randomized reflections from crystal platelets surrounded by optically clear amorphous regions and solid-gas interfaces at surfaces and within the polymer matrix. A three-dimensional distribution of reflected photons is not uniform. That is, reflected light intensity is not equal upon a hemisphere surrounding a photon impact point and is also a function of the AOI. Manufactures of diffuse reflective surface welcome randomized dispersion and have not recognized a need for any repeating nanostructure. Reducing randomness or exploiting inherent non-uniformity would allow improved shadowing in diffuse reflective systems. Manufactures embrace Lambertian reflectance which disperses photons with about a 0° AOI such that reflected photons are more concentrated perpendicular to the surface and more rarified as the angle approaches 90°.

According to Fischer¹, “ . . . at the near-specular direction at high angles of incidence even [sintered PTFE] is not a very good depolarizer, retaining some of the preference for scattering s-polarization in the specular direction.” According to Janecek,² “All Lambertian reflectors that were measured had a high (>98%) Lambertian component at low incidence angles (<50°), but a specular component appeared at high incidence angles.” Fig. PHO-03 illustrates Janecek's findings. X-axis 0310 is the angle of incidence. Y-axis 0320 and its complimentary-Y-axis 0325 provide a fraction of Lambertian and specular reflection respectively. A Lambertian fraction of reflected light and its specular complement are plotted as four curves for four generally diffuse reflective materials. These four materials and their respective curves are glossy Teflon® material 0330, 4 layers of ACER hardware Teflon® tape 0340, 3 layers of ACER hardware Teflon® tape 0350, and titanium dioxide paint 0360. Specular reflection increases at the expense of diffuse reflection as the angles of incidence get larger and larger. Figure PHO-03 provides for angles of incidence between about 0° and up to about 80° to 86°. Diffuse reflection is greatest at about 0° to 40°. As AOI increases further diffuse reflection yields to specular reflection down to at least about 86°. Reliable measurements at angles greater than the low to mid-80s were not possible with Janecek's instrument. ¹Optical System Design, 2^{nd} Edition, p. 534.²M. Janecek, “Reflectivity Spectra for Commonly Used Reflectors,” IEEE Transactions on Nuclear Science, vol. 59, no. 3, pp. 490-497, June 2012, doi: 10.1109/TNS.2012.2183385.

### Residence Time

Residence time is a foggy concept for a device that continuously or semi-continuously processes recycled air. In any PES occupied by protectees, targets are continuously introduced into the air and hence PES air is unlikely to be target-free. The true measure of photonic APS target chamber performance is a product of the number of photons created a fraction of those photons that strike pathogens, and photonic lethality. This is a critical design consideration as target chamber volume, and hence size, is not a determinant of performance. System performance is determined almost exclusively by photonic efficiency. Photonic efficiency is most predominantly determined by an APS target chamber reflectance. Of course, as previously described, flight path length is a factor and hence-size does matter.

To illustrate, consider 100 targets in an entirely enclosed space. Assume that fluidic residence time required to destroy a desired level of target inside of a given target chamber is 2 seconds. It may make little difference if a pathogen passes through the target chamber twice for 1 second on each pass or once for 2 seconds. In the former case the air flow rate is twice as high as the latter case and hence the residence time is half, but in the same total elapsed time (assuming complete mixing of the room air) any given number of the target (e.g., 50, 90, 99) are rendered harmless. The two cases may thus be nearly identical. However, neutralizing a virus quickly and overwhelmingly is superior to a slow death. In the latter case it is possible that a treatment will be mutagenic to the virus without entire loss of viability. While most pathogen mutations are harmless to a protectee, there is a small chance that a mutation will improve a pathogen's ability to infect or increase its lethality.

An advantageous way to express system performance is the time it takes to reduce targets in a PES by an order of magnitude (1 log reduction). Put another way, a device that has a 5-log single pass reduction but would require 12 hours to achieve a 1-log reduction in the room in which it operates is 12-times less effective than a device that has a 1-log single pass reduction and requires 1 hour to achieve a 1 log reduction in the same room.

A superior choice includes a high single pass APS target chamber lethality and rapid target reduction rates in a PES.

### Isentropic Compression-Expansion Machine (ICE Machine)

The building blocks of life including all pathogenic organisms are proteins and nucleic acids. Both are denatured with modest temperature increases. Volatile organic compounds (VOCs) in the presence of oxygen in air are oxidized primarily to carbon dioxide and water. Flash pasteurization is routinely practiced denaturing pathogens in foodstuffs. Thermally induced oxidation of T1K and oxidizable T3K in air conventionally involves first heating air entraining targets and then cooling the pasteurized mixture. Even if an air-to-air heat exchanger is utilized to recover a portion of the energy the process remains energy intensive.

Some microorganisms including but not limited to nematodes, protozoa, and bacteria, are inactivated by non-atmospheric pressure and pressure changes without oxidation playing a substantive role. Studies show that temperature has an influence on the extent of pressure induced inactivation. Isentropic induced lysis (IIL) is lysis initiated by isentropic compression, an optional isochoric reaction period, and a subsequent isentropic expansion. Isentropic compression necessarily involves a temperature increase, therefore IIL may occur alongside and be complimentary to isentropically induced oxidation (IIO).

In one embodiment of the present invention nearly isentropic compression-expansion (ICE) induces oxidation and/or lysis and overcomes an energy inefficiency of conventional pasteurization by recovering at least a portion of the potential energy stored when air is pressurized. For brevity we refer to these as ICE machines.

For disinfection of air as contemplated in the present invention, it is apparent that the amount of fuel (i.e., T1K and T3K) in a typical PES is extremely dilute or ELF concentration. Together T1K and T3K are typically much less than 1000 ppm, generally less than 100 ppm and often less than 10 ppm. Under the right conditions of temperature and pressure T1K and oxidizable T3K autoignite, but such low concentrations will not propagate a macro scale flame. For a flame to propagate in a macro volume a heat of oxidation from an initial oxidation site at a micro-scale (e.g., an air-born virus or an individual volatile molecule) must provide enough energy to an adjacent T1K aerosol or oxidizable T3K gas molecule to propagate a flame. Flame propagation is not possible where the lowest lower flammability limit of flammable vapors and dusts is about 8,000 ppm and a concentration of T1K plus oxidizable T3K is at least 8 times lower than an expected lower flammability limit. More often two or three orders of magnitude separate a concentration of T1K plus oxidizable T3K and their combined lower flammability limit. Under such circumstances macro flame propagation is not possible. A condition where a concentration of flammable T1K and oxidizable T3K in air is at least five-times lower than a lower flammability limit is referred to herein as extremely low fuel or ELF.

Nano-scale flame propagation is believed to occur with T1K. As non-limiting examples, a typical respiratory virus such as influenza and corona have diameters of about 100 nm. The amino acids and nucleic acids that make up such viruses include predominantly carbon and hydrogen atoms in not fully oxidized states and are known to readily begin oxidation below about 200° C. to 280° C. When, for example, a first amino acid of a virus spontaneously oxidizes upon heating to 240° C., the heat of combustion of that oxidation raises the temperature of adjacent organic molecules accelerating the oxidation of these neighbors. This propagation may extend across and within the 100 nm scale of the virus. Thus, at nano scale flames propagate, but at a macro scale of a MVV, flames are not likely to propagate.

### Generic ICE MVV

A non-limiting example of a 4-cylinder integrated cycle ICE machine is illustrated in two perspective views in Figs. ICE-01A and ICE-01B. In Fig. ICE-01B snapshot 20120 flywheel 20141 has advanced about 180° from Fig. ICE-01A snapshot 20110. Cylinder 20101, cylinder head 20106, and piston 20111 comprise a first MVV. Cylinder 20102, cylinder head 20107, and piston 20112 comprise a second MVV. Cylinder 20103, cylinder head 20108, and piston 20113 comprise a third MVV. Cylinder 20104, cylinder head 20109, and piston 20114 comprise a fourth MVV. Each cylinder head has at least one intake valve and at least one exhaust valve. Each piston is sealingly engaged with each cylinder with a dynamic pneumatic sealing means such as at least one piston ring, O-ring, D-ring, or other dynamic pneumatic sealing means well known in the art. Piston 20111 is sealingly engaged with cylinder 20101 with sealing means 20196. Piston 20112 is sealingly engaged with cylinder 20102 with sealing means 20197. Piston 20113 is sealingly engaged with cylinder 20103 with sealing means 20198. Piston 20114 is sealingly engaged with cylinder 20104 with sealing means 20199. Cylinder head 20106 includes intake valve 20161 and exhaust valve 20162. Cylinder head 20107 includes intake valve 20171 and exhaust valve 20172. Cylinder head 20108 includes intake valve 20181 and exhaust valve 20182. Cylinder head 20109 includes intake valve 20191 and exhaust valve 20192. Other valve arrangements are possible without departing from the spirit of the present invention.

Intake valve 20161 is actuated by valve actuator 20165 via valve connection 20163. Intake valve 20171 is actuated by valve actuator 20175 via valve connection 20173. Intake valve 20181 is actuated by valve actuator 20185 via valve connection 20183. Intake valve 20191 is actuated by valve actuator 20195 via valve connection 20193.

Exhaust valve 20162 is actuated by valve actuator 20166 via valve connection 20164. Exhaust valve 20172 is actuated by valve actuator 20176 via valve connection 20174. Exhaust valve 20182 is actuated by valve actuator 20186 via valve connection 20184. Exhaust valve 20182 is actuated by valve actuator 20186 via valve connection 20184. Exhaust valve 20192 is actuated by valve actuator 20196 via valve connection 20194.

Piston 20111 is connected to crank shaft 20140 by a piston rod 20151. Piston 20112 is connected to crank shaft 20140 by a piston rod 20152. Piston 20113 is connected to crank shaft 20140 by a piston rod 20153. Piston 20114 is connected to crank shaft 20140 by a piston rod 20154. Referring to Fig. ICE-01A snapshot 20110, piston 20111 is beginning an intake stroke, piston 20112 is beginning a compression stroke, piston 20113 is beginning an exhaust stroke, and piston 20114 is beginning an expansion stroke. When those strokes have completed flywheel 20141 has advanced about 180° and piston 20111, piston 20112, piston 20113, and piston 20114 illustrated in Fig. ICE-01B snapshot 20120 are each beginning, compression, expansion, intake, and exhaust strokes respectively. Intake valves on each cylinder are closed except during an intake period which largely coincides with an intake stroke. Exhaust valves are closed except during an exhaust period which largely coincides with an exhaust stroke. Analogous to a 4-stroke internal combustion piston engine, an ICE machine includes at least one MVV (e.g., volume enclosed by cylinder 20101, cylinder head 20106, piston 20111 and sealing means 20196), at least one intake valve (e.g., intake valve 20161), at least one exhaust valve (e.g., exhaust valve 20162), and a means (e.g., piston rod 20151) to transfer force between an at least one MVV and at least one shaft (e.g., crank shaft 20140). Said at least one intake valve is actuated to allow gaseous communication between designated air from flow path 10134 illustrated in Fig. APS-01A to an MVV. Said at least one exhaust valve is actuated to allow gaseous communication between a MVV to provide polished air into flow path 10135 illustrated in Fig. APS-01A.

One embodiment of an ICE machine includes an optional flywheel (e.g., flywheel 20141) to store kinetic energy. Figs. ICE-01A and ICE-01B illustrate these features. In one embodiment of an ICE machine valves may be actuated by a camshaft or may use a controller (i.e., a microprocessor operating with a suitable program), in a manner known in the art, to provide electronic control of a valve assembly, and a valve-assembly under such circumstances may include, for example, a solenoid-operated valve that is responsive to a controller.

Fig. ICE-02 illustrates the thermodynamic relationships between static compression-expansion ratio and the temperature and pressure of air together with entrained T1K and oxidizable T3K as a function of a static compression/expansion ratio from 1 to 30. Expansion is the inverse of compression. The static compression-expansion ratio is represented by X-axis 20210. The left Y-axis 20220 and solid line 20250 illustrate the relative change in pressure (P₂/P₁). The right Y-axis 20230 and dashed line 20260 show temperature in a compressed state (T2) in Celsius. The dotted line 20270 illustrates the approximate equilibrium temperature of a well-insulated adiabatic MVV. Inlet air temperature and pressure are 27° C. and 101 kPa respectively. As non-limiting examples, typical reciprocating gasoline engines and Wankel rotary engines have static compression ratios of about 8-10, typical diesel engines have static compression ratios of about 15-20, and rotary cam-guided engines have static compression ratios of at least 12 to 25. See U.S. Pat. No. 9,810,068B2, “Rotary engine with cam-guided rotor,” ¶101. While Fig. ICE-02 illustrates static compression ratios up to 30 on X-axis 20210, higher static compression/expansion ratios enjoy certain advantages and disadvantages and examples illustrated by Fig. ICE-02 do not limit the scope of the present invention. Piston engines and pistonless rotary engines of many designs including but not limited to Otto cycle, diesel, Atkinson cycle, Wankel, LiquidPiston (cam-guided rotary), Engineair, Hamilton Walker, Libralato rotary Atkinson cycle, quasiturbine, RKM, Sarich orbital, Trochilic, Wave disk, nutating disk, gerotor, IRIS (radial impulse), turbines, and others all include isentropic compression, the addition of a fuel, and a power-decompression step. All could be modified to an ICE machine by one skilled in the art by eliminating the addition of fuel and substituting an isentropic expansion step for the power-decompression step without departing from the spirit of the present invention.

Y-axis 20230 of Fig. ICE-02 is annotated with eight example threshold temperatures at atmospheric pressure. Seven callouts 20231 through 20237 illustrate examples of oxidation and/or denaturation at atmospheric pressure. Callout 20240 delineates the onset of undesirable compound formation at atmospheric pressure. At atmospheric pressure and above about 1200° C., compounds of nitrogen and oxygen (NO_{X}) form. Callouts 20231, 20232, 20233, 20235, 20236, and 20237 provide six examples of T3K, at their atmospheric pressure degradation or autoignition temperatures. A group of common amino acids, callout 20234, are destroyed over a range of temperatures as shown by bracket 20239. Amino acids are constituents of all T1K and decompose endothermically between 185° C. and 280° C. at atmospheric pressure. See Weiss I M, Muth C, Drumm R, Kirchner H O K, “Thermal decomposition of the amino acids glycine, cysteine, aspartic acid, asparagine, glutamic acid, glutamine, arginine and histidine,” BMC Biophys. 2018 Feb. 9; 11:2. doi: 10.1186/s13628-018-0042-4. PMID: 29449937; PMCID: PMC5807855.

The synergistic combination of increased pressure and increased temperature act together to simultaneously decrease the autoignition temperature and decrease the autoignition delay time providing for efficient IIO at temperatures, pressures and elapsed times that are less than predicted by reported autoignition temperatures and autoignition delay times at atmospheric pressure.

Still referring to Fig. ICE-02, pressure increases at compression ratios from 1 to 30 as delineated by X-axis 20210 are depicted by line 20250. For an example static compression ratio of 10, the pressure increases about 25.1-times atmospheric, and temperature increases from about 27° C. to about 481° C. as depicted by line 20260. These are peak values. Both temperature and pressure increase in a compression stroke until they reach about these maximums near a minimum volume of an MVV. Both peak values then decrease in a subsequent expansion stroke to approximately their starting values. Time (seconds) required to complete each stroke is the quotient of 60 sec/min and twice an ICE machine's rotational speed (RPM). Doubling of the denominator results from the two strokes each piston completes on each revolution of an exemplary ICE machine illustrated in Fig. ICE-01. For example, for an ICE machine operating at 1000 RPM, each stroke would last 0.03 seconds and the total time that air and targets are at temperatures and pressures above their ambient values is about 0.06 seconds. About half during a compression stroke and about half during an expansion stroke.

There are two mechanisms by which targets are neutralized. For biological targets (i.e., T1K), proteins, nucleic acids, and other chemical structures required for viability become damaged by higher temperatures and in some cases higher pressures. See Brown P, Meyer R, Cardone F, Pocchiari M., “Ultra-high-pressure inactivation of prion infectivity in processed meat: a practical method to prevent human infection,” Proc Natl Acad Sci USA. 2003 May 13; 100 (10): 6093-7. doi: 10.1073/pnas. 1031826100. Epub 2003 May 5. PMID: 12732724; PMCID: PMC156331. This neutralization does not require that a T1K be fully oxidized, but rather that it loses its viability—its ability to reproduce or infect. Loss of viability is a function of time dependent temperature and pressure profiles. A second mechanism is extensive or complete oxidation. This second mechanism addresses both T1K and oxidizable T3K.

Fig. ICE-03 is an exemplary temperature and pressure profile covering 0.06 seconds (60 milliseconds) displayed on X-axis 20310 of a compression stroke 20312, a isochoric dwell 20313, and an expansion stroke 20314 for the ICE machine illustrated in Fig. ICE-01 operated at about 1000 RPM. Dotted line 20340 is a static compression ratio; dotted line 20350 is a pressure ratio. Both are measured on left Y-axis 20320. Solid line 20360 is a temperature profile and is measured on right Y-axis 20330 along with example autoignition and deactivation temperatures for T1K and T3K at atmospheric pressure. Y-axis 20330 is annotated with six examples of degradation or autoignition temperatures at atmospheric pressure. Actual autoignition temperatures are lower than illustrated because actual pressure in a MVV is above atmospheric. VOCs ricin 20372, mustard gas 20373, jet A-1 fuel 20374, ethanol 20375, and xylene 20376 autoignite over a range of about 80° C. to about 470° C. Amino acids 20370 are a group of the most common amino acids. At atmospheric pressure these amino acids are constituents of all T1K and decompose endothermically between 185° C. and 280° C. indicated by bracket 20371 to create mostly water, some ammonia, hardly any carbon dioxide, and no NO_{X}. (See Weiss I M, Muth C, Drumm R, Kirchner H O K. Thermal decomposition of the amino acids: glycine, cysteine, aspartic acid, asparagine, glutamic acid, glutamine, arginine, and histidine. BMC Biophys. 2018 Feb. 9; 11:2. doi: 10.1186/s13628-018-0042-4. PMID: 29449937; PMCID: PMC5807855.) The ammonia thus formed burns in air to form nitrogen and water if ammonia's autoignition temperature is exceeded. (See Tudela, “Ammonia-Air Mixtures Can Be Explosive,” J. Chem. Ed., V. 76, N. 4, April 1999, p. 468. The atmospheric auto-ignition temperature of ammonia is about 651° C. An ICE machine can be configured to provide a peak temperature of over 700° C. with a static compression ratio of 20:1 or a peak temperature of about 900° C. with a static compression ratio of 30:1. Even higher static compression ratios and temperatures are advantageous for certain applications.

A maximum static compression ratio may be practically limited by formation of NO_{X} compounds above about 1204° C. at atmospheric pressure or even lower temperatures at higher pressures. Thermal NO_{X} production increases with the square root of operating pressure. (See Richards et al, “Combustion Strategies for Syngas and High-Hydrogen Fuel,” Elevated levels of nitrogen dioxide can cause damage to a protectee's respiratory tract and increase a protectee's vulnerability to, and the severity of, respiratory infections and asthma. Long-term exposure to high levels of nitrogen dioxide can cause chronic lung disease. A recommended air quality standard for nitrogen dioxide is 0.053 ppm for an annual exposure period.

Judiciously choosing the static compression ratio and an ICE engine's rate of revolution controls the elapsed time that air and entrained T1K and oxidizable T3K remain above a second temperature and a second pressure for a period longer than an autoignition delay time. Said second temperature is below a peak temperature, but above targeted autoignition temperature and pressure, decomposition temperatures, or denaturation temperatures for a desired elapsed time greater than an autoignition delay time. A desired temperature, pressure and elapsed time are chosen to achieve a desired decomposition of T1K and oxidizable T3K and to remain below a targeted level of NO_{X}.

A second method to achieve a desired elapsed time utilizes serial processing of air through more than a single MVV. A plurality of ICE machines can be placed in series such that a common exhaust of a machine communicates with an inlet to at least a second machine.

In a third method, an exhaust from an individual MVV may communicate with an inlet of another individual MVV. For example, in Fig, ICE-01A, exhaust from cylinder 20103 may be directed from valve 20182 via a manifold to valve 20161 and into cylinder 20101.

A fourth method to achieve a desired elapsed time is to employ a repeatable compression-expansion stroke pair, bookended with intake and exhaust strokes as represented by Fig. ICE-04. The arrows illustrate a cycle order, “I” represents an intake stroke, “C” represents a compression stroke, “Exp” represents an expansion stroke, “Exh” represents an exhaust stroke, and “N” is a natural number signifying compression-expansion stroke pairs. For example, if N is 3, a sequence of repeating strokes would be:

I→C→Exp→C→Exp→C→Exp→Exh

and an elapsed time within compression-expansion stroke pairs is increased by a factor of three compared to a case where N is 1.

A fifth configuration and method to achieve a desired elapsed time may by realized by reconfiguring at least one pair of MVVs into a split-cycle which will be described in Isochoric Split Cycle ICE Machine.

Once one of the five configurations described is chosen together with required parameters, an ICE machine may be configured to treat a required volume of air by adjusting the number of MVV APSs and/or by adjusting dimensions of individual MVVs. A non-limiting example is a 4-liter ICE machine with four cylinders operated at 1000 RPM where air passes through a single cylinder before it is exhausted. This 4-liter, 4-cylinder ICE machine provides 2000 liters per minute (70.6 CFM) of treated air. The air delivery rate scales about one-to-one with the number of cylinders, revolutions per unit time, and a volume of each cylinder. It is generally beneficial to have multiple cylinders of the same geometry so forces are balanced with time, but it would not be a departure from the present invention to utilize different cylinder displacements.

The isentropic nature of an ICE machine allows energy required to compress air to be largely recovered in an expansion stroke. Energy requirements are primarily the result of frictional losses in moving parts of an ICE machine, work required to blow air through ducts and associated filtration stages, and entropy increases resulting from small departures from ideal isentropic operation.

One such departure from ideal isentropic operation is illustrated in Fig. ICE-02 by line 20270. Line 20270 is an approximate steady-state equilibrium temperature of an ICE machine's MVV components for each compression/expansion ratio delineated by X-axis 20210 and where N=1 in Fig. ICE-04. A steady-state adiabatic equilibrium temperature of the MVV components (e.g., piston, cylinder, cylinder head, valves, and crankcase) is approximated by equation 2.

$$[ N \cdot ( {T}_{2} + {T}_{1} ) / 2 + {T}_{1} ] / ( N + 1 ) \qquad ( 2 )$$

Where T2 is the isentropic maximum temperature 20360 in Fig. ICE-03, T₁ is an ambient air temperature, and N is a natural number signifying the number of compression-expansion stroke pairs as illustrated by Fig. ICE-04. The first part of the numerator of equation 2, the leftmost product, sum and quotient, represents the average temperature of gases in an ICE machine MVV during compression and expansion strokes. An exemplary temperature profile with time is graphed in Fig. ICE-03. During intake and exhaust strokes the gas temperature within a MVV is about T₁. For N=1, half of an elapsed time is at about T₁ and half of an elapsed time is at about the average of T₁ and T₂. Where a repeatable compression-expansion stroke pair are utilized (i.e., N>1), a time spent at an average of T₁ and T₂ is extended by N times. While each stoke is essentially adiabatic and isentropic during start-up there is an average temperature difference between gases confined within a MVV and an MVV itself. A small net amount of heat energy is thus transferred from the gases to a MVV until an average temperature of both is about the same and equilibrium is established. An adiabatic equilibrium is possible if a MVV is thermally insulated. The adiabatic equilibrium temperature is approximately represented by line 0270 in Fig. ICE-02. For example, for a compression/expansion ratio of 10 and N=1, the equilibrium temperature is about 254° C. In a first embodiment of the present invention, MVVs are insulated to yield an equilibrium temperature substantially above ambient.

In a second embodiment of the present invention heat loss from a MVV in encouraged by not including insulation and optionally augmenting heat loss from above ambient temperatures utilizing methods well known in the art including passive or active air cooling and/or liquid cooling. With utilization of modest cooling means the temperature of the MVV can be held to below 3° C. above T₁, below 6° C. above T₁, below 9° C. above T₁, or below 12° C. above T₁.

Fig. ICE-05 illustrates a relationship between isentropic energy and isentropic temperatures in comparison to actual energy and temperatures for a 1-liter MVV which is provided cooling to hold it about 3° C. above an ambient temperature. The small difference between isentropic energy and temperature and actual energy and temperature for a MVV provided with cooling means to yield a MVV temperature about 3° C. above an ambient temperature and where a heat transfer coefficient between air within a MVV and a MVV enclosure is about 0.0015 J/(cm²·K·s). X-axis 20510 presents elapsed time in seconds from the beginning of a compression stroke 20512 to the end of a subsequent expansion stroke 0514 of an individual 1-liter cylinder of a 4-cylinder ICE machine as illustrated in Fig. ICE-01 with a 10:1 static compression ratio. Against Y-axis 20540 a lossless isentropic energy 20560 of about 1.11 liters of air is plotted as a solid line and its actual energy line 20570 is plotted as a long-dash line. The two lines are difficult to distinguish because the maximum deviation from isentropic behavior at 0.06 seconds is about 2.9 joules or 1.7% of an air kinetic energy at an end of an expansion stroke. For a 4 liter, 4-cylinder engine a total thermodynamic energy loss is about 97 watts.

Isentropic air temperature 20580 and actual air temperature 20590 are plotted on Y-axis 20550. At 0.06 seconds a maximum deviation of about-5.2° C. occurs. At a peak temperature of a compression-expansion stroke pair at 0.03 seconds, an actual temperature difference is about 2.7° C. At MVV temperatures 6° C., 9° C., and 12° C. above ambient an energy loss is about 1.7%, 1.6%, and 1.5% respectively and a maximum temperature deviation at 0.06 seconds is 5.0° C., 4.8° C., and 4.6° C. respectively. This second embodiment of an ICE machine (i.e., not insulated and employing MVV housing cooling means) enjoys a lower operating temperature. A lower operating temperature accrues two advantages over higher temperature options.

First, a lower temperature places lower oxidative demands on lubricants used to lubricate MVV moving parts. Longer oil life reduces maintenance requirements and reduces formation of lower molecular weight and more volatile decomposition by-products.

Second, a lower temperature decreases vapor pressure of volatile components of a lubricant including lubrication degradation components. During intake and compression strokes a first portion of lubricant and lubricant vapors introduced at sealing surfaces between a compression chamber interior and exterior (e.g., piston rings, apex seals and the like) may be oxidized in compression and expansion strokes and have no materially adverse consequences. A second portion of a lubricant and lower molecular weight lubricant degradation products introduced near the end of an expansion stroke and during an exhaust stroke may be problematic. High molecular weight, low vapor pressure lubricant in this second portion largely remains on the MVV internal surfaces of a low temperature second embodiment. However, a portion of lower molecular weight, volatile lubricant components and degraded lubricant components are VOCs and may partially evaporate and exit with exhaust. An introduction of trace VOCs may create undesirable odors and is counter to the goal of generating clean air.

Such trace lubricant VOCs may optionally be passed through an adsorption media or an absorber to remove them. Non-limiting examples of such media are well known in the art and include activated carbon and zeolites. A lower temperature of a MVV lowers the rate that lubricant VOCs will be created, and lowers their evaporation rate, near the end of an expansion stroke and during an exhaust stroke.

A third embodiment of the present invention utilizes an appropriate combination of insulation and/or active and/or passive cooling to reach any steady-state temperature of an ICE machine MVV between a value slightly greater than T1 and about an equilibrium temperature 20270 of Fig. ICE-02.

The required energy to power an ICE machine may be provided by torque source 20142. Torque source 20142 may include, but is not limited to an electric motor, a human or animal driven crank, or a power-take-off (a.k.a., PTO) from a motorized vehicle. Appropriate low volatile, low temperature lubricants may be used to minimize frictional losses. Heat generated from friction, a small portion of heat of compressed air with each compression stroke, and perhaps heat loss from a torque source if integral to the ICE machine will warm an engine above ambient. Heat is removed continuously by ambient air being treated. An ICE machine will warm when it is turned on from a cold start until it reaches an equilibrium when a thermal energy output is about equal to an energy input. The ICE machine may optionally be cooled with any method well known in the art.

### Rotary ICE Machine

Previously enumerated “engines” have been cited as examples of machines that can create compressed volumes suitable for ICE machines. A non-limiting example taken from the long list of internal combustion designs, U.S. Pat. No. 9,810,068B2, describes a cam-guided rotary engine and a 2-3 lobe gerotor that creates “very long dwell” times. (See paragraph of [0236] of U.S. Pat. No. 9,810,068B2: “A 2-3 lobe rotor may be used as well, but it creates a very long dwell (a constant volume duration).”) Modifications of these designs are particularly well suited for an ICE machine application. In the absence of reciprocating parts (e.g., pistons, piston rods, and valves), a cam-guided rotary engine and a gerotor enjoy nine advantages.
1. create less noise,
2. induce less vibration,
3. reduce energy consumption,
4. have fewer moving parts,
5. are more compact,
6. require considerably less mass,
7. attain a higher static compression ratio,
8. can be configured to supply variable static compression ratios, and
9. can be configured to prolong a compressed state dwell time.

These nine advantages accrue to an analogous cam-guided rotary ICE machine. Comparing the ICE machine illustrated in Fig. ICE-01 to an analogous cam-guided rotary engine or a gerotor, stripping ignition and cooling systems reduces complexity (advantage 4), size (advantage 5), and mass (advantage 6) even further. The maximum isentropic pressure shown in Fig. ICE-02 is a maximum pressure that an ICE machine must be designed to withstand. This contrasts with any analogous internal combustion engine which introduces fuel, ignition, and a confined explosion. An explosion results in a pressure spike greater than an isentropic maximum pressure. An ICE machine requires less tensile strength than a comparable internal combustion engine with the same static compression ratio. The absence of an explosive pressure spike yields additional reductions in noise (advantage 1), vibrations (advantage 2), size (advantage 5), and mass (advantage 6).

The benefits of advantages 1-3 are self-evident. The benefits that result from advantage 4 are reduced manufacturing costs, longer life, and lower maintenance costs. The benefits from advantages 5 and 6 are a more diminutive footprint than would otherwise be required and less effort to move an ICE machine. A benefit of advantage 7 is an ability to reach higher temperatures and pressures to induce oxidation and/or lysis in targets. A benefit of advantage 8 is an ability to tune performance to address specific targets and specific use cases. The benefit of advantage 9 is best understood by referring to Fig. ICE-06 and comparing it to Fig. ICE-03. Fig. ICE-03 shows the static compression ratio, pressure, and temperature profiles over 0.06 seconds for an isentropic compression and expansion of air in a piston-cylinder arrangement such as that illustrated in Fig. ICE-01.

Fig. ICE-06 illustrates the same maximum static compression ratio of 10 but in a cam-guided rotary engine. Analogous to Fig. ICE-03, Fig. ICE-06 provides an exemplary temperature and pressure profile covering 0.06 seconds (60 milliseconds) displayed on X-axis 20610 of compression stroke 20612, isochoric dwell 20613, and expansion stroke 20614 of a cam-guided rotary ICE machine. The duration of a compression stroke 20612 is about 0.023 seconds. The duration of an isochoric dwell 20613 is about 0.014 seconds. The duration of an expansion stroke 20614 is about 0.023 seconds. Curve 20640 is a static compression ratio; curve 20650 is a pressure ratio. Both are measured on Y-axis 20620. Curve 20660 is a temperature profile and is measured on Y-axis 20630 along with example autoignition and deactivation temperatures for T1K and oxidizable T3K at atmospheric pressure. Y-axis 20630 is annotated with six examples of degradation or autoignition temperatures at atmospheric pressure. Actual autoignition temperatures are lower than illustrated because actual pressure in the MVV is above atmospheric. VOCs ricin 20672, mustard gas 20673, jet A-1 fuel 20674, ethanol 20675, and xylene 20676 autoignite over a range of about 80° C. to about 470° C. Amino acids 20670 are a group of the most common amino acids. At atmospheric pressure these amino acids are constituents of all T1K and decompose endothermically between 185° C. and 280° C. indicated by bracket 20671 to create mostly water, some ammonia, and hardly any carbon dioxide and almost no NO_{X}.

The difference between static compression ratio profile 20340 of a piston-type machine illustrated in Fig. ICE-03 and static compression ratio profile 20640 of a cam-guided rotary-type machine illustrated in Fig. ICE-06 is the latter's extended isochoric dwell period 20613 that spans about 0.014 seconds compared to the former's brief isochoric dwell period 20313 that spans about 0.003 seconds. In the example illustrated by Fig. ICE-06, that extra 0.011 seconds comes at the expense of compression stroke 20612 and expansion stroke 20614 which relinquish about 0.0055 seconds each. While the example illustrates a symmetrical compression and expansion stroke, such symmetry is not required. A cam-guided rotary engine can be designed to execute a non-symmetrical isentropic compression/isochoric dwell/expansion cycle. In some circumstances it might be advantageous to accelerate a temperature and pressure increase of a compression portion of a cycle and to retard a decrease during an expansion portion of a cycle. The opposite may also be true. In most cases it is advantageous to maximize isochoric dwell.

As a non-limiting example, the benefit of lengthening an isochoric portion of a profile of an isentropic compression-expansion cycle can be appreciated by integrating areas under the temperature and pressure curves of Figs. ICE-03 and ICE-06 and above an isentropic action condition (IAC). An exemplary IAC for denaturation of amino acids is about 244° C. and about 7-times atmospheric pressure. Referring first to Fig. ICE-03, IAC temperature 20380 demarcates a lower temperature bound of about 244° C. and area 20385 (shaded with waves) represents an integral of a temperature difference between temperature profile 20360 and IAC temperature 20380. IAC pressure 20381 demarcates a lower pressure bound of about 7-times atmospheric pressure and area 20386 (shaded with dots) represents an integral of a pressure difference between pressure profile 20350 and IAC pressure 20381.

Referring now to Fig. ICE-06, IAC temperature 20680 demarcates a lower temperature bound of about 244° C. and area 20685 (shaded with waves) represents an integral of a temperature difference between temperature profile 20660 and IAC temperature 20680. IAC pressure 20681 demarcates a lower bound of about 7-times atmospheric pressure and area 20686 (shaded with dots) represents an integral of a pressure difference between pressure profile 20650 and IAC pressure 20681. A comparison of the areas illustrated in Figs. ICE-03 and ICE-06 demonstrate the temperature integral over time for the cam-guided rotary engine is about 2.4-times larger than for a piston engine equivalent at the same static compression ratio. Similarly, a pressure integral over time for a cam-guided rotary engine is about 2.7-times larger than for a piston engine equivalent at the same static compression ratio. Both higher temperature and higher pressure decrease both autoignition temperature and autoignition delay time, and both higher temperature and higher pressure accelerate IIL for T1K, the resulting multiple and synergistic advantages of extended isochoric dwell can be leveraged to improve at least one of the following:
1. increase a log reduction of T1K and oxidizable T3K,
2. lower a static compression ratio and an associated temperature and pressure,
3. conserve energy,
4. decrease ICE machine size, and/or
5. increase ICE machine throughput by increasing RPM.

### Isochoric Split-Cycle ICE Machine

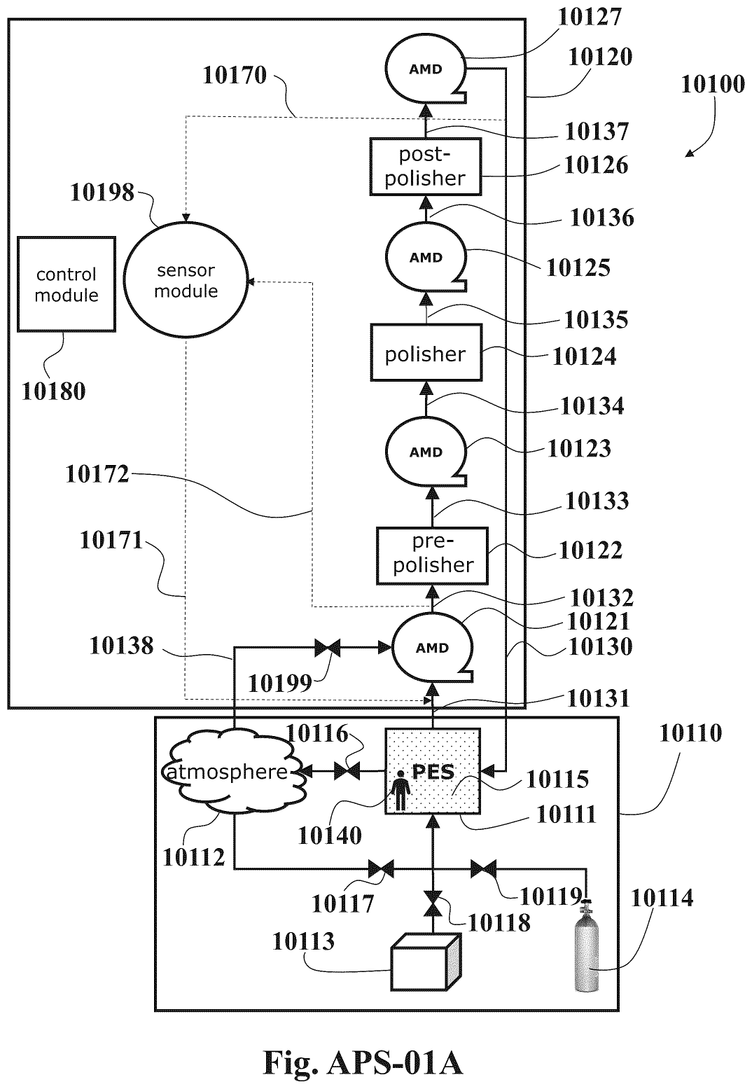

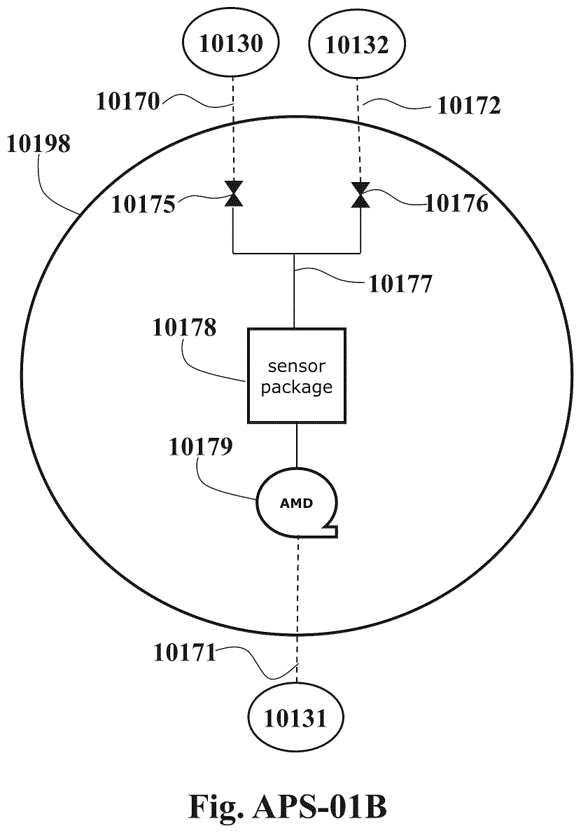

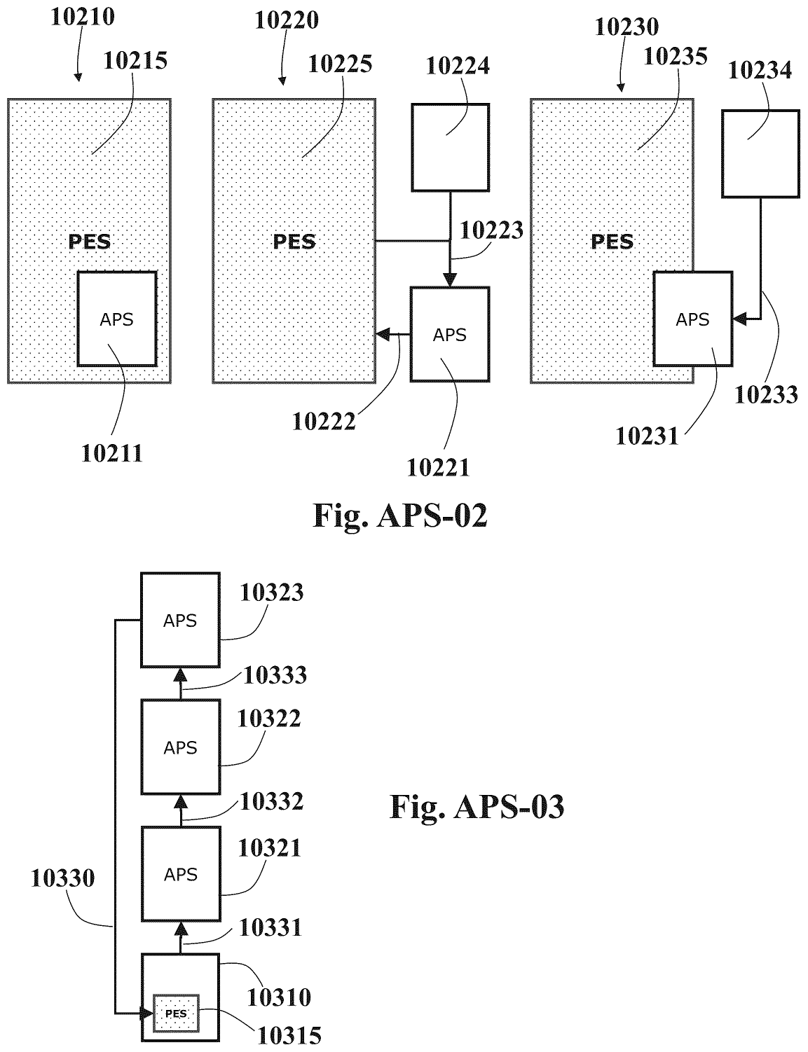

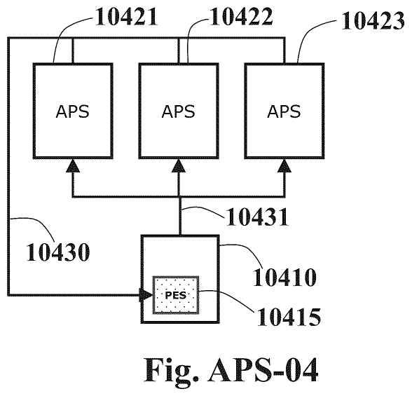

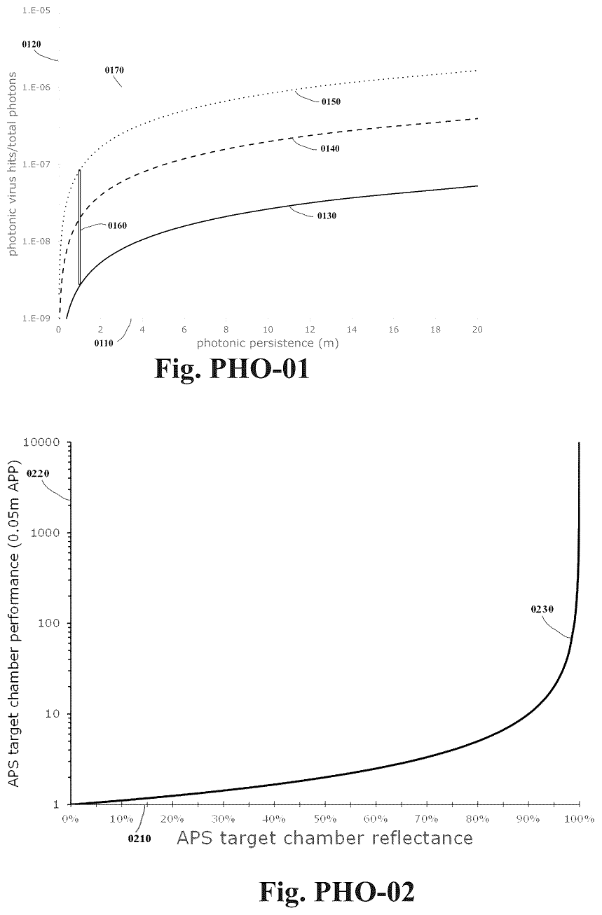

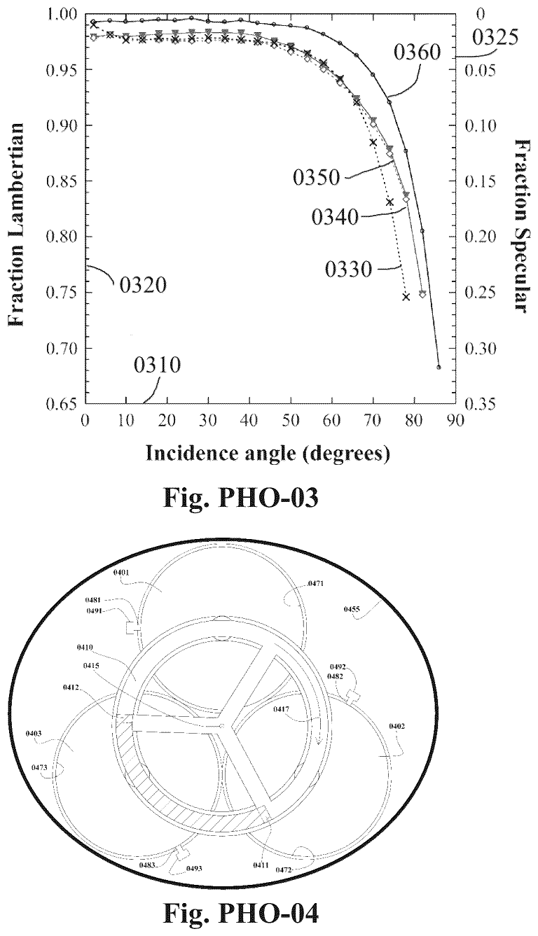

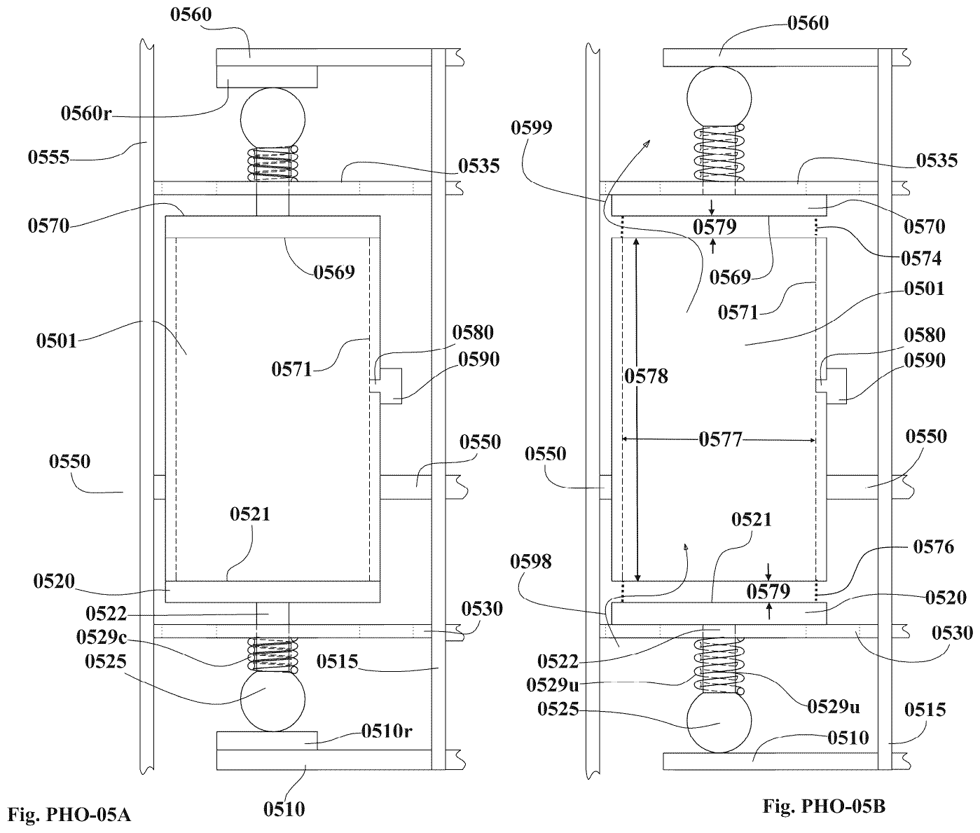

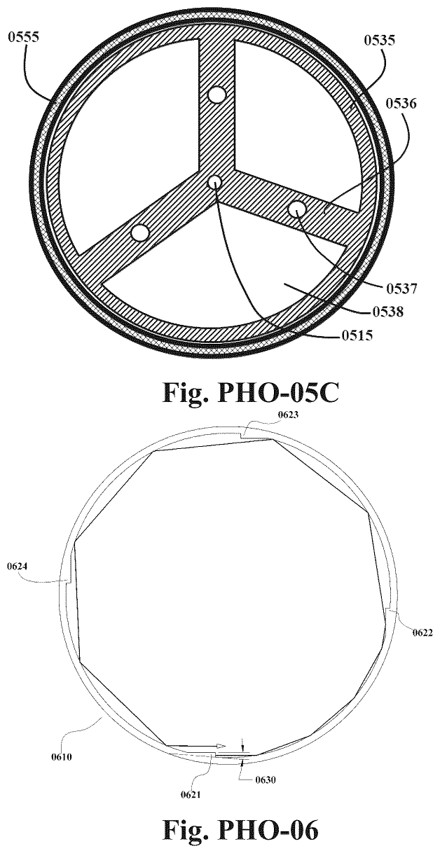

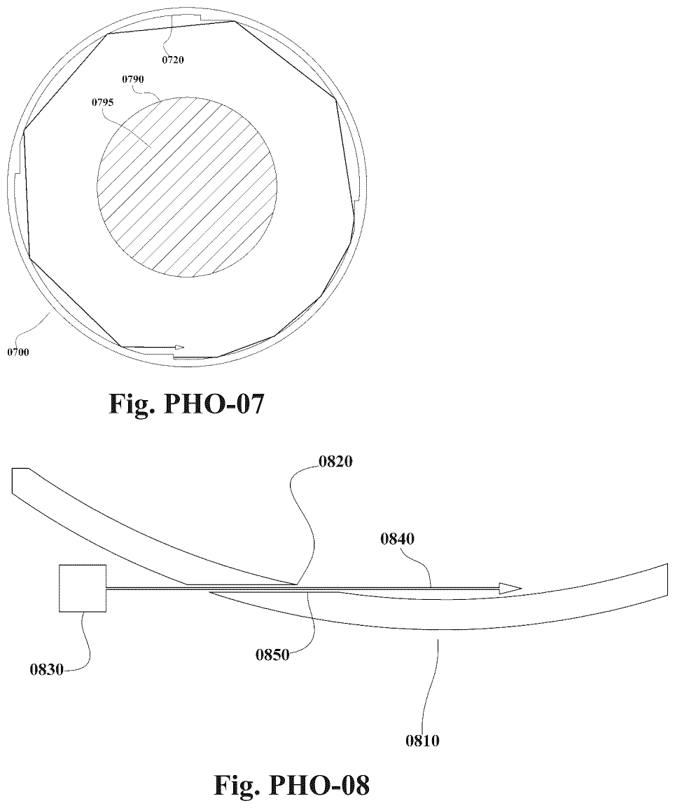

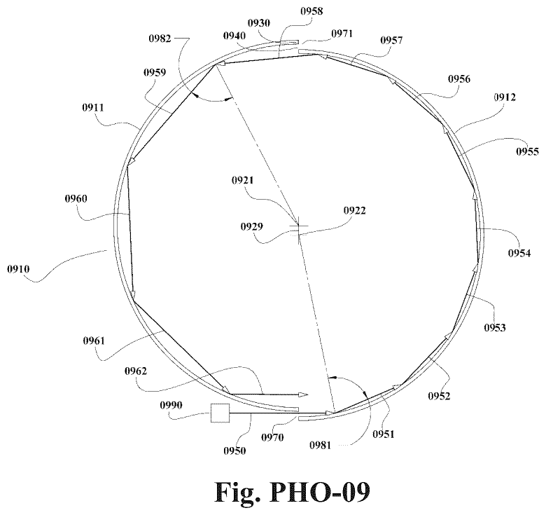

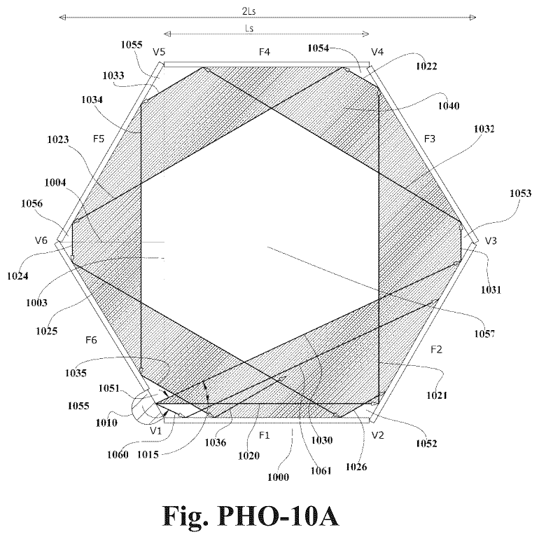

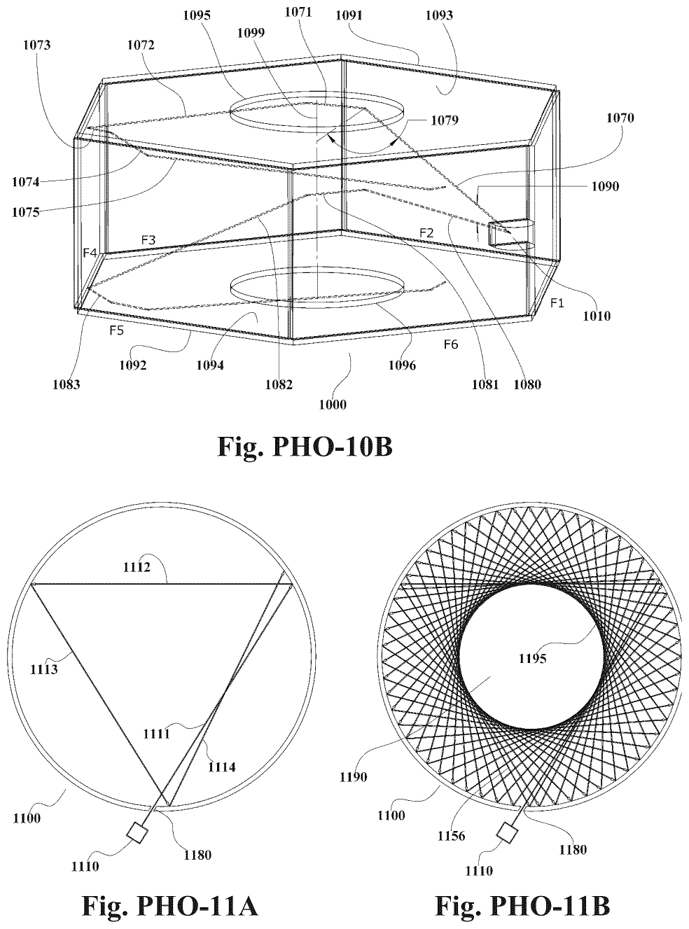

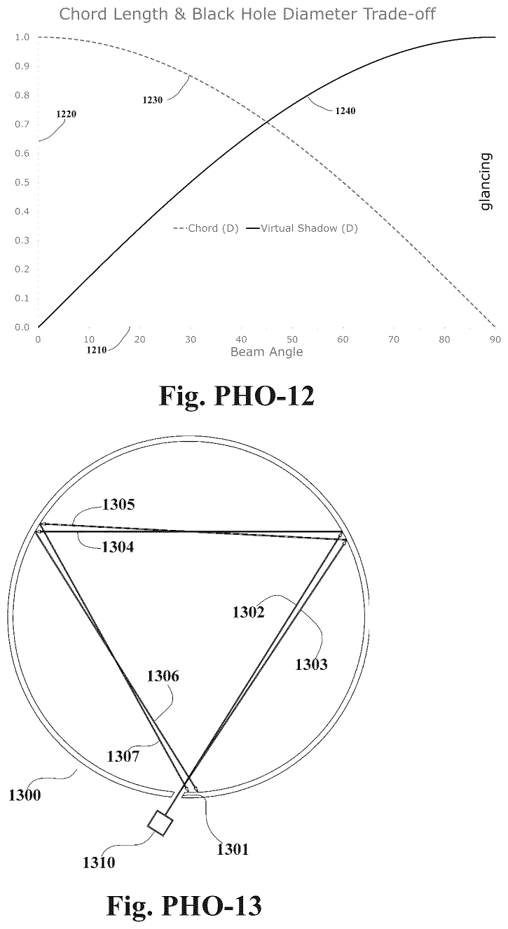

A compressed state dwell time can be increased further by employing another embodiment of the present invention, namely a split-cycle. Fig. ICE-07 provides a simplified example of a split-cycle ICE machine. Fig. ICE-07 is a modification of Fig. 3-1 of U.S. Pat. No. 8,844,473. Split-cycle ICE machine 20700 illustrated in Fig. ICE-07 includes at least four valve-valve actuators 20718, 20719, 20728, and 20729, at least two for each cylinder and located on or near compressor head 20711 and expander head 20721. Valve and valve actuator assembly 20718 is closed except during an intake stroke of compressor 20710. Valve and valve actuator assembly 20719 is closed except near the end of a compression stroke of compressor 20710. Valve and valve actuator assembly 20729 is closed except it opens near the beginning of an expansion stroke of expander 20720. Valve and valve actuator assembly 20728 is closed except it opens during an exhaust stroke of expander 20720. An actuator portion of a valve-valve actuator assembly (e.g., 20718, 20719, 20728, and 20729) may incorporate at least one of a mechanical cam actuator, a hydraulic actuator, a pneumatic actuator, a steam actuator, and an electric actuator. A valve portion of a valve and valve actuator assembly (e.g., 20718, 20719, 20728, and 20729) includes nearly airtight seals when closed and may include aerodynamic smoothing of sealing surfaces to facilitate airflow with a modest pressure drop. Such valves are well known in the art of internal combustion engines.

Oxidizer 20750 is isochoric, adiabatic, isobaric, and essentially isothermal.

In a diesel-like cycle, air together with T1K and oxidizable T3K, designated air 20705, are drawn into compressor cylinder 20710 on a previous downward stroke of piston 20730a through at least a first compressor intake valve-valve actuator 20718. Maximum volume occurs when volume 20715 is filled and piston rod 20732 is in its lowest position attached to crankshaft 20734. Near maximum volume the at least first compressor intake valve-valve actuator 20718 closes, and crank shaft 20734 rotates through its next 180° driven in part by expander 20720 and in part by torque source 20790 to isentropically compress air in MVV compressor 20710.

As piston 20730a travels toward cylinder head 20711 pressure and temperature increase as illustrated in Fig. ICE-02 by line 20250 and line 20260 respectively. Split-cycle ICE machine 20700 and split-cycle ICE machine 20900 (Fig. ICE-09) may advantageously depart from a design constraint of integrated cycle ICE machine 20800 (Fig. ICE-08). MVVs of integrated cycle ICE machines must have a non-zero minimum volumes; split-cycle ICE machines advantageously employ zero minimum volume MVVs or a ZMV-MVV. As a non-limiting example consider MVV 20101 of integrated ICE machine 20110 illustrated in Fig. ICE-01A.

The clearance between cylinder head 20106 and piston 20111 at minimum volume determines a static compression ratio of MVV 20101. For this example, the static compression ratio is 10. For a 1-liter displacement integrated cycle MVV the minimum volume is one-tenth of 1 liter or 0.1 liters. If the minimum volume was 0.05 liters, the resulting static compression ratio would be 20. As the minimum volume is reduced further and further the compression ratio increases until at a minimum volume of zero, a theoretical compression ratio would be infinite. Of course, infinite compression ratios are not possible and are not desirable for the present invention. In contrast, a split cycle MVV accrues an unexpected advantage by employing a ZMV-MVV. Referring to compressor MVV 20710 in Fig. ICE-07, a clearance 20712 is defined by the position of piston 20730a and cylinder head 20711. The volume of MVV 20710 at any time is the product of clearance 20712, pi and the square of the radius of piston 20730a. If clearance 20172 is zero, the volume of MVV 0710 is zero and absent an opening of valve-valve actuator 20719 the pressure would be infinite. In the present invention valve-valve actuator 20719 opens when a desired dynamic compression ratio is achieved even as clearance 20712 continues toward zero. Valve-valve actuator 20719 may be driven by a conventional cam in which case a single compression ratio is chosen by design. A preferred embodiment of the present invention uses a variable actuation mechanism to actuate valve-valve actuator 20719 at a moment controlled by controller 20795 to realize at least one operational advantage.

A split cycle ZMV-MVV can support an infinitely variable dynamic compression ratio from 1 to any desirable positive value. A first exemplary advantage of a variable dynamic compression ratio is an ability to tune a split-cycle MVV compression ratio to remove specific targets. As a non-limiting example and referring to Fig. ICE-02, carbon monoxide (CO), target 20237, requires considerably higher temperature to oxidize compared to jet A-1 fuel, target 20233. Where carbon monoxide is not present a split-cycle ICE machine may be operated at a lower compression ratio and thus have commensurately more output at the same RPM. If carbon monoxide is detected the compression ratio may be increased to reduce that target. Referring to Fig. ICE-07 optional sensor module S4 20798 (analogous to sensor module 10198 in Figs. APS-01A and APS-01B) measures the concentration of targets using methods well known in the art. For the present example, sensor module S4 20798 measures a concentration of carbon monoxide in at least designated air 20705 and is in wired or wireless communication with controller 20795. Preferably sensor module S4 20798 also measures a concentration of carbon monoxide in polished air 20785 to confirm the efficacy of ICE machine 20700. While it is within the scope of the present invention to have two carbon monoxide sensors, a first measuring CO concentration in designated air 20705 and second measuring CO concentration in polished air 20795, a single sensor that samples toggling air streams from both is particularly advantageous as controller 20795 can calculate a differential concentration which is largely insensitive to sensor drift.

Optional sensor package S4 20798 may include at least one sensor including but not limited to carbon monoxide concentration, NO_{X} concentration, temperature, pressure, humidity, carbon dioxide concentration, particulate count and size distribution, VOC concentration, biological assays, oxygen concentration, or any concentration of any analyte.

A second exemplary advantage of a controllable compression ratio is the ability to reduce the dynamic compression ratio if the formation of NO_{X} is too high. It is also possible that a compromise must be chosen to balance two opposing goals. For example, in the presence of very dangerous carbon monoxide a higher dynamic compression ratio is called for and if doing so increases NO_{X} formation, controller 20795 may make a trade-off in the best interest of at least one protectee in a PES. Controller 20795 preferably includes a human interface to warn protectees of less-than-ideal designated air and/or polished air.

A third exemplary advantage of the present invention is the ability to purposefully ramp compression ratio to a target compression ratio when ICE machine 20700 is required to change from a lower compression ratio to a higher compression ratio. For example, when ICE machine 20700 is turned on from a cold start a compression ratio is to be increased from 1 to a higher value. Proceeding slowly from a lower value to a higher value minimizes torque requirements on torque source 20790 and minimizes mechanical and thermal stresses on ICE machine 20700. Ramping compression ratio from a starting value to a target value reduces noise.

At steady state as compressor outlet valve-valve actuator 20719 opens the pressure and temperature on both sides of valve-valve actuator are about the same. Piston 20730a continues as clearance 20712 approaches zero. The volume 20715 approaches zero and isentropically compressed gas flows into conduit 20740 and into isochoric oxidizer 20750. Oxidizer 20750 and conduits 20740 and 20770 are constructed to hold compressed gas without leaking. Preferably the oxidizer 20750 and conduits 20740 and 20770 are thermally insulated to operate essentially adiabatically and isothermally.

With optional modifications ICE machine 20700 may be used to cool air in addition to its primary purpose of removing at least a portion of T1K and oxidizable T3K. A first optional modification includes omitting at least a portion of the thermal insulation to allow a portion of the thermal energy of the hot-compressed air to escape passively outside of a PES. To minimize the impact on the primary purpose of ICE machine 20700 the omission of insulation is best done as far downstream in the flow path from conduit 20740, oxidizer 20750, and conduit 20770. Put another way, insulation is better omitted nearest valve-valve actuator 20729 and furthest from valve 20719. Heat lost from the flow path (conduit 20740, oxidizer 20750, and conduit 20770) reduces the energy of the gas provided to expander 20720. Consequently, torque source 20790 must provide more torque to compensate. Gas exhausted to 20785 will be cooler than it otherwise would have been proportionally with the extent of heat loss.

A second optional modification of ICE machine 20700 adds an active heat exchanger 20771 to cool air in conduit 20770 with cooling media 20775 flowing in conduit 20772. Nonlimiting examples of suitable heat exchanger 20771 include double tube (i.e., tube in tube), shell and tube, and plate types. Cooling media 20775 may be an open loop source such as the atmosphere or water from the sea, a lake, or a stream, or may include a closed loop source such as a heat transfer fluid. Where a heat transfer fluid in a closed loop is utilized, said heat transfer fluid may be cooled in a second heat exchanger such as but not limited to a radiator commonly used for internal combustion engines. A great deal of cooling is possible with this second optional modification without significantly detracting from the primary purpose of ICE machine 20700. As with the passive first optional modification, more torque must be supplied by torque source 20790 to provide the desired cooling effect.

During a start-up phase of operation, the pressure and temperature in the isochoric oxidizer chamber and connecting conduits possess close to ambient values and hence it takes some number of compressor 20710 strokes to bring the pressure and temperature in the oxidizer 20750 and associated conduits 20740 and 20770 up to about the pressure and temperature corresponding to the static compression ratio of MVV compressor 20710. After this pressurization and warm-up period is complete, the oxidizer is essentially adiabatic, isobaric, isothermic, and isochoric.

The isochoric oxidizing chamber 20750 may be sized to yield any desired compressed state dwell time. As a non-limiting example, a dwell time in a 10-liter oxidizer (including ancillary conduits) in a 4 liter, 4-cylinder, 10:1 compression ratio split-cycle ICE machine operating at 1000 RPM is about 2.7 seconds, 810-times longer than a conventional piston-cylinder-type MVV. Dwell time scales directly with an oxidizer volume. For example, doubling an oxidizer plus ancillary conduit volume to 20 liters results in a 5.4 second dwell time—a 1620-fold improvement compared to a conventional piston-cylinder-type MVV.

The oxidizer 20750 is preferably designed to have a minimal pressure drop and to assure each volume of air delivered to oxidizer 20750 minimally mixes with previously or subsequently supplied air volumes. In a preferred embodiment each quantum of air or target remains within the oxidizer for about an equivalent time to encourage uniform target treatment time. Non-limiting examples include serpentine oxidizer 20751 of tubing or a flow laminizer in a larger diameter generally cylindrical oxidizer. Many other shapes are possible without departing from the spirit of the invention. While some mixing or even complete mixing within the oxidizer is not a preferred mode of operation, such a mode remains within the scope of the present invention.

Oxidizer 20750 may optionally provide variable dwell time. In a non-limiting example, at least one pair of three-way valves 20752 and 20753 may be installed at a fraction of a length of serpentine oxidizer 20751. Three-way valve 20752 may be connected to three-way valve 20753 which is in fluid communication with conduit 20770. Toggling said at least one valve pair changes oxidizer 20750 dwell time at a given RPM of torque source 20790 and crankshaft 20734. In the first position the entirety of serpentine oxidizer 20751 is utilized. In the second position a fraction of serpentine oxidizer 20751 is bypassed. There are many other ways to plumb at least one bypass to adjust dwell time in oxidizer 20750 which remain in the spirit of the present invention.

The oxidizer 20750 may also include at least one catalyst to facilitate oxidation of certain targets at lower temperature and/or shorter dwell times. A lower temperature reduces NO_{X} formation.

The observed reduction in NO_{X} in catalytic combustors is much greater than expected from the lower combustion temperature. A reaction on a catalytic surface produces little or no NO_{X} directly. (See Meherwan P. Boyce, in Gas Turbine Engineering Handbook (Fourth Edition), 2012. Non-limiting examples of appropriate catalyst include Ni, Ni/Mg, Ru, Pd, and Pt.

After a dwell time in conduit 20740, oxidizer 20750, and conduit 20770 air and oxidation products of T1K and oxidizable T3K are fed through at least a first open expander intake valve-valve actuator 20729 into volume 20725 within expander 20720 when the piston 20730b is close to its minimum volume. The minimum volume is preferably zero. During steady state operation, expander intake valve-valve actuator 20729 closes when a designated decompression ratio of expander 20720 is equal to a designated expansion ration of compressor 20710. Piston 20730b is driven isentropically downward by essentially the same force it takes to drive piston 20730a upward on its compression stroke. The small difference in the two forces is caused by (1) the cumulative pressure drops of the oxidizer 20750 and conduits 20740 and 20770, (2) the thermal losses from the same components, and (3) frictional losses between cylinders and pistons and in the bearings of rotating hardware (i.e., piston rods 20732, crankshaft 20734, mechanical valve actuators (if present), and the like). When piston 20730b reaches about its maximum volume an at least first expander exhaust valve-valve actuator 20728 opens and as piston 20730b rises it exhausts a volume 20725 through exhaust conduit 20780 to polished air 20785 which is in airtight fluid communication with flow path 10135 of Fig. APS-01A at essentially ambient temperature and pressure. Polished air 20785 is different from designated air 20705 in that there is less T1K and oxidizable T3K and more water vapor, carbon dioxide, and nitrogen, and a slightly higher temperature. The increase of water vapor, carbon dioxide, and nitrogen results from oxidation of that portion of T1K and oxidizable T3K that is less than found in designated air 20705 provided by flow path 10134 in Fig. APS-01A. The slightly higher temperature is a result of frictional losses in moving parts, pressure losses from gas flow, and departures from ideal isentropic conditions.

In a first exemplary embodiment of the present invention volume 20725 of expander 20720 share a displacement volume and a minimum volume as the displacement and minimum volumes of compressor 20710. The displacement volumes are both about 1 liter and minimum volumes are about zero liters. Compressor 20710 operates with a dynamic compression ratio of about ten.

Expander 20720 operates with a dynamic expansion ratio of about ten. When piston 20730b has completed an exhaust stroke and exhaust valve-valve actuator 20728 closes, volume 20725 is zero. A moment later expander intake valve-valve actuator 20729 opens and compressed air at about 25.1-times higher pressure flows from conduit 20770 into volume 20725. Each volume of air which flows through valve-valve actuator 20729 into volume 20725 isentropically and isobarically drives expander piston 20730b away from expander cylinder head 20721 until expander intake valve 20729 closes. At steady state expander intake valve-valve actuator 20729 closes when the mass of air in volume 20725 is equal to the mass of air displaced from volume 20715. After expander intake valve-valve actuator 20729 closes expander piston 20730b continues away from expander cylinder head 20721 isentropically and not isobarically. Except for small mechanical and thermodynamic losses, the forces exerted on piston 20732a and on piston 20732b are about the same magnitude but with opposite signs and hence approximately cancel each other out in net force to crank shaft 20734.

To the extent it is advantageous for compressor intake valve-valve actuator 20718 and expander intake valve-valve actuator 20729 to protrude respectively into compressor volume 20715 and expander volume 20725, as respective pistons and cylinder heads begin to depart from zero clearance, the valves open with about the same linear velocity as respective pistons. Analogously for compressor exhaust valve-valve actuator 20719 and expander exhaust valve-valve actuator 20780 may protrude respectively into compressor volume 20715 and expander volume 20725, as respective pistons and cylinder heads approach zero clearance, the valves close with about the same linear velocity as respective pistons. Thus a ZMV-MVV is not constrained by valves that protrude into an MVV volume.

In a second exemplary embodiment of the present invention volume 20725 of expander 20720 share the same displacement volume and minimum volume as the displacement and minimum volumes of compressor 20710. The displacement volumes are both about 1 liter and the minimum volumes are both about 0.11 liters. When piston 20730b has completed an exhaust stroke and exhaust valve-valve actuator 20728 closes, volume 20725 is at a minimum value and the pressure is about equal to atmospheric pressure. A moment later expander intake valve-valve actuator 20729 opens and compressed air at about 25.1-times higher pressure rushes from conduit 20770 into volume 20725. For a first volume of air which flows through valve-valve actuator 20729 into volume 20725 its pressure spikes rapidly downward, a negative pressure spike, to about 1 atmosphere and then returns moments later to about 25.1-times atmospheric. Subsequent volumes experience a similar negative pressure spike, but the magnitude of the spike diminishes with each volume passing through valve-valve actuator 20729. At the same time pressure in conduit 20770 decreases. The magnitude of the decrease depends on the geometry of conduit 20770. These momentary pressure reductions attenuate along conduit 20770 as the distance from valve-valve actuator 20729 increases. Such rapid pressure changes have three notable effects:
1. Lysis of biological organisms and biological macromolecules occurs from pressure changes, especially rapid pressure changes. T1K ICE machine performance may be amplified.
2. Isentropic performance of an ICE machine suffers from entropy increases when such pressure changes occur without recovering a commensurate quantity of work. An undesired consequence of an entropy increase is greater energy demands upon torque source 20790.
3. Rapid changes in pressure may create undesired noise.

Volume 20725 of expander 20720 need not have the same minimum volume as the minimum volume of compressor 20710. In a second exemplary embodiment of the present invention volume 20725 of expander 20720 has a lessor minimum volume than the minimum volume of compressor 20710. For example, displacement volumes are both about 1 liter, the minimum volume of compressor 20710 is about 0.11 liter, and the minimum volume of expander 20720 is about zero. When piston 20730b has completed an exhaust stroke and exhaust valve 20728 closes, volume 20725 is zero and pressure is about equal to atmospheric pressure. A moment later expander intake valve-valve actuator 0729 opens and compressed air from conduit 20770 at about 25.1-times higher pressure begins applying that pressure on piston 20730b at about the same moment that piston 20730b begins its expansion stroke. To maintain close to isentropic performance valve-valve-actuator 0729 is maintained open by controller 20795 until the product of about 0.11 liters of air, a temperature ratio T₂/T₁, and a pressure ratio P₁/P₂ has been delivered into volume 20725. T₁ is the absolute temperature measured by S1 T-P sensor 20747. T₂ is the absolute temperature measured by S2 T-P sensor 20777. P₁ is the absolute pressure measured by S1 T-P sensor 20747. P₂ is the absolute pressure measured by S2 T-P sensor 20777. If pressure drop through oxidizer 20750 and its ancillary conduits 20740 and 20770 is negligible, S1 sensor 20747 and S2 sensor 20777 may omit pressure measurement as the ratio of P₁/P₂ is unity. Controller 20795 is in wired or wireless communication with S1 T-P sensor 20747, S2 T-P sensor 20777, S3 position sensor 20799 and with valve-valve actuator 20729. Controller 20795 makes the required calculation in near real time and sends This second embodiment does not enjoy a potential benefit (effect 1) of additional lysis but avoids the energy losses (effect 2) and noise (effect 3) of the first embodiment.

Intermediate values of the difference between a compressor minimum volume and an expander minimum volume are within the scope of the present invention allowing designers to choose a trade-off between a desirable lysis amplification (effect 1) and undesirable energy inefficiency (effect 2), and undesirable noise (effect 3).

While Fig. ICE-07 illustrates a single pair of cylinders 20736 comprised of compressor 20710 and expander 20720, a preferred design includes even integer pairs of cylinders or other MVVs. 2, 4, 6, 8, et cetera MVV pairs, or 4, 8, 12, 16, et cetera MVVs respectively. Even integer cylinder pairs enjoy an advantage of a close to perfect balance of forces. It is desirable to temporally balance the force of a compression stroke with that of an expansion stroke such that the net torque at the crankshaft is close to zero. The diesel-like cycle described includes 4 cycles covering two revolutions of a crank shaft, even integer MVV pairs enables the simultaneous execution of at least one paired compression and expansion stroke on each 180° rotation of a crankshaft. A net torque of close to zero enjoys three advantages over an integer number of MVVs which are not even integer pairs.
1. Minimization or even elimination of a need for a flywheel which yields size, weight, cost, and complexity savings.
2. Reduction of noise and vibration as the torque source provides continuous, steady power to overcome frictional losses.
3. The near simultaneous opening of at least one exhaust valve near the end of a compression stroke and near minimum volume on a first compression MVV and an opening of at least one intake valve near an end of an exhaust stroke and near a minimum volume of a second expander MVV allows a near isobaric flow of compressed gases from a compressor, through an oxidizer and its ancillary conduits, and into an expander. While a first compression MVV and a second expansion MVV are in their respective intake and exhaust strokes, a second compression MVV and a first expansion MVV are in their respective compression and expansion strokes. Thus, for each half-revolution of a crankshaft at least one compressor MVV is compressing, at least one expander MVV is expanding, at least one compressor MVV is intaking air, at least one expander MVV is exhausting air, and the net torque is always close to zero. Absent this feature the pressure in the oxidizer would necessarily pulsate creating variable mechanical stress and thermodynamic losses on the oxidizer and ancillary conduits. Variable stress shortens the life of equipment, generates undesirable noise from pressure pulsation, and results in variable torque requirements of the torque source.

The flexibility afforded by the split-cycle ICE machine derives from an ability to create virtually any desired dwell time or even variable dwell time. Longer dwell times may be leveraged to:
1. increase log reductions in targets,
2. lower static compression ratios which lower temperature and pressure profiles, or
3. a compromised combination of 1 and 2.

Another flexibility afforded by the split-cycle ICE machine results from the potential to introduce at least one catalytic surface to an oxidizer. Such catalysts can accelerate oxidation to allow greater throughput or to operate at a lower compression ratio.

Isochoric oxidizer 20750 is preferably well insulated to minimize thermodynamic losses and allow ICE machine 20700 to operate isentropically, adiabatically, and isothermally. In a first embodiment of the present invention temperature management of cylinder pair 20736 (comprised of compressor 20710 and expander 20720) may by allowed to warm as a thermally integrated pair in the same way as integrated cycle ICE-machine illustrated in Fig. ICE-01A. In a thermally integrated pair compressor 20710 and expander 20720 share about the same temperature. Optional thermal isolator 20795 is not present and heat transfer between compressor 20710 and expander 20720 is not discouraged. Compressor 20710 and expander 20720 may share a common cylinder block, a common crankcase, and common lubricant 20791 and 20792. Absent thermal barrier 20795, lubricant 20791 and lubricant 20792 have an essentially identical composition and temperature.

In a second embodiment of the present invention compressor 20710 and expander 20720 may be thermally isolated from each other with thermal isolator 20795. Compressor 20710 and expander 20720 may not share a common cylinder block, an integrated crankcase, and lubricants. In this second embodiment optional thermal isolator 20795 is present and lubricant 20791 and lubricant 20792 are not in fluid communication, are not in intimate thermal contact, and may be of different chemical composition. It may be advantageous to allow the temperature of compressor 20710 to increase to a value higher than the temperature of expander 20720. Put another way, expander 20720 enjoys enhanced expander cooling compared to compressor 20710. Compressor 20710 may operate essentially adiabatically, or more modest cooling may be used such that a temperature of compressor 20710 is greater than a temperature of expander 20730. At least two advantages accrue to this temperature difference.

First, allowing compressor 20710 to operate at higher temperature is more thermodynamically efficient thus requiring less torque from torque source 20790. A higher temperature of compressor 20710 results in more volatile components (i.e., VOCs) from mechanical lubricants to vaporize into volume 20715. Said VOCs may be carried into oxidizer 20750 where they will be oxidized. The introduction of these VOCs from compressor 20710 does not materially interfere with the operation of ICE machine 20700.

Second, operating expander 20720 at a lower temperature minimizes release of VOCs from mechanical lubricants into volume 20725, before volume 20725 cools during adiabatic expansion stroke. The lessor quantity of VOCs derives from two mechanisms. First, oxidation and other degradation mechanisms of a lubricant occur more slowly at lower temperatures. Consequently, fewer volatile components are produced. Second, the vapor pressure of all lubricant components goes down with temperature. A lessor quantity of volatile components is released at about atmospheric temperature and pressure by valve assembly 20728 and are transported by flow path 20780 and become polished air 20785 which is in airtight communication with flow path 10135 of Fig. APS-01A.

Heat loss from a MVV compressor and/or expander may be encouraged by not including insulation and optionally augmenting heat loss from above ambient temperatures including passive or active air cooling and/or liquid cooling. With utilization of modest cooling means well known in the art of internal combustion engines the temperature of a MVV can be held to below 3° C. above ambient air temperature, below 6° C. above ambient air temperature, below 9° C. above ambient air temperature, or below 12° C. above ambient air temperature.

Isochoric split-cycle machine 20700 illustrated by Fig. ICE-07 employs pistons and cylinders for MVVs. The present invention is not limited by this geometry. Piston engines, pistonless rotary engines, and turbines of many designs including but not limited to Otto cycle, diesel, Atkinson cycle, Wankel, LiquidPiston (cam-guided rotary), Engineair, Hamilton Walker, Libralato rotary Atkinson cycle, quasiturbine, RKM, Sarich orbital, Trochilic, Wave disk, nutating disk, gerotor, IRIS (radial impulse), turbines, and others all include an isentropic compression, an addition of a fuel, and a power-decompression step. Without departing from the spirit of the present invention these combustion engine designs could be adapted by one skilled in the art by eliminating the addition-of-fuel step, adding an isochoric oxidation chamber outside of at least one MVV, substituting an isentropic expansion step for a power-decompression step, and adding a torque source.

### Pneumatic Considerations

While any natural number of MVVs falls within the scope of ICE machines, a preferable number of MVVs for integrated cycle and split-cycle ice machines is a natural number which is evenly divisible by 4. When there are 4 MVVs acting in concert as illustrated in Fig. ICE-01A at any arbitrary moment a first MVV is in an exhaust stroke and in fluid communication with flow path 10135 in Fig. APS-01A and a second MVV is in an intake stoke and in fluid communication with flow path 10134 in Fig. APS-01A. Third and fourth MVVs are in compression and expansion strokes with valves closed and are not in fluid communication with anything. The balance of air flow in and out of a 4-MVV ICE machine provides smoother air flow as will be demonstrated.

Pneumatic flow paths for exemplary integrated cycle and split-cycle quad-MVVs are illustrated in Figs. ICE-08 and ICE-09 respectively. Fig. ICE-08 illustrates air flows in integrated cycle quad MVV 20800 including MVV 20801, MVV 20802, MVV 20803, and MVV 20804. Each MVV includes at least one intake valve in fluid communication with intake header 20810 which contains designated air and is in airtight fluid communications with air duct 10134 of Fig. ICE-01 and at least one outlet valve in fluid communication with exhaust header 20820 which contains polished air and is in airtight fluid communications with air duct 10135 of Fig. ICE-01. MVV 20801 receives designated air from intake valve 20861 and discharges polished air from exhaust valve 20862. MVV 20802 receives designated air from intake valve 20871 and discharges polished air from exhaust valve 20872. MVV 20803 receives designated air from intake valve 20881 and discharges polished air from exhaust valve 20882. MVV 20804 receives designated air from intake valve 20891 and discharges polished air from exhaust valve 20892.

Fig. ICE-09 illustrates air flows in split-cycle quad MVV 20900 including MVV 20901, MVV 20902, MVV 20903, and MVV 20904. Compressor MVVs 20901 and 20903 include at least one intake valve in fluid communication with intake header 20910 which contains designated air and is in airtight fluid communications with air duct 10134 of Fig. ICE-01. Expander MVVs 20902 and 20904 include at least one outlet valve in fluid communication with exhaust header 20920 which contains polished air and is in airtight fluid communications with air duct 10135 of Fig. ICE-01. Compressor MVVs 20901 and 20903 include at least one exhaust valve in fluid communication with pre-oxidizer header 20940 which carries compressed air to oxidizer 20950. Oxidizer 20950 provides extended dwell time for compressed air. After compressed air has passed through oxidizer 20950 it flows into post-oxidizer header 20970. Post-oxidizer header 20970 is in fluid communication with expanders 20902 and 20904 via their respective intake valves 20971 and 20991. MVV 20901 receives designated air from intake valve 20961 and discharges compressed air from exhaust valve 20962. MVV 20902 receives compressed air from intake valve 20971 and discharges polished air from exhaust valve 20972. MVV 20903 receives designated air from intake valve 20981 and discharges compressed air from exhaust valve 20982. MVV 20904 receives compressed air from intake valve 20991 and discharges polished air from exhaust valve 20992.

Fig. ICE-10 illustrates approximate air flow 21060 measured on Y-axis 21040 for single exemplary integrated cycle piston-cylinder MVV 20103 of Figs. ICE-01A and ICE-01B as a function of time on X-axis 21010 during exhaust stroke delincator 21012 and vacuumed into MVV 20103 of Fig. ICE-01A during intake stroke delincator 21014. MVV 20103 of Fig. ICE-01B is a piston-cylinder MVV with a 1-liter displacement, a 10:1 static compression ratio, a bore of about 10.46 cm, a stroke of about 11.63 cm, a crank radius of about 5.8 cm, a piston rod length of about 12.2 cm, and is rotating at about 1000 RPM. At the beginning of exhaust stroke 21012 piston 20113 is located as shown in Fig. ICE-01A and MVV 20103 is at maximum volume. Piston 20113 is also not moving as it is reversing direction and air flow is about zero. In the next instant piston 20113 moves toward head 20107 and reaches maximum velocity about 0.018 seconds later at point 21080. At about the same moment air flow reaches its maximum of about 58 liters per second at point 21080. As piston 20113 (Fig. ICE-01B) continues flow slows and approaches zero as MVV 20103 (Fig. ICE-01B) approaches minimum volume at about point 21090. Three other things happen at about point 21090: exhaust valve 20172 (Fig. ICE-01B) closes, intake valve 20171 (Fig. ICE-01B) opens, and intake stroke 21014 begins. Intake stroke 21014 is essentially a mirror image of exhaust stroke 21012. For simplicity, Fig. ICE-11 ignores intake strokes, but the principles for an intake stroke mirror those of an exhaust stroke.

Fig. ICE-11A, ICE-11B and ICE-11C include X-axis 21110. X-axis 21110 is elapsed time over a 0.12 second period encompassing 4-strokes and 2 complete crankshaft revolutions. Y-axis 21140 references exhaust air in an exhaust header for an ICE machine utilizing an integrated cycle or a split-cycle. For example, in ICE-08 header 20820 carries polished air away from 4-MVV integrated cycle polisher 20800 and in ICE-09 header 20920 carries polished air away from 4-MVV split-cycle polisher 20900. ICE machine polishers may have any natural number of MVVs.

MVV 20103 of Fig. ICE-01B is a piston-cylinder MVV with a 1-liter displacement, a 10:1 static compression ratio, a bore of about 10.46 cm, a stroke of about 11.63 cm, a crank radius of about 5.8 cm, a piston rod length of about 12.2 cm, and is rotating at about 1000 RPM. Exhaust manifold flow rate 21113 for this single piston-cylinder MCC is illustrated in Fig. ICE-11A. A puff of about 1 liter of polished air is exhausted over 0.03 seconds and no more polished air is exhausted for about the 0.09 seconds required for a single piston to complete its three non-exhaust strokes. For some applications puffed polished air flow is not an issue and single a MVV, a double MVV, or a three-MVV configuration is within the scope of the present invention.

Where protectees are near an ICE machine, multiple cylinders provide a more continuous source of polished air and a reduction in resulting staccato created by abruptly varying air flow. Fig. ICE-11B shows the combined polished air flows 21113, 21114, 21112, and 21111 of four MVVs 20103, 20104, 20102, and 20101 respectively arranged as illustrated in Fig. ICE-01B. While this four-MVV configuration eliminates extended periods of zero flow, the flow does approach zero and the difference between the maximum and maximum flow rates is about 58 liters per second.

Further improvement in continuity of flow and a more legato sound may be achieved by adding another set of four MVVs bringing the total MVVs to eight. As illustrated in Fig. ICE-11C, four new MVVs are designated as MVV-5, MVV-6, MVV-7, and MVV-8 and independently contribute polished air flow to an exhaust header as polished air flow 21115, 21116, 21117, and 21118 respectively. The four new MVVs are operated 90-degrees out of rotational phase with the first set of four MVVs. Total polished air flow 21180 is the sum of the exhaust air flows for all eight MVVs as a function of time. Of course, doubling the number of MVVs from four to eight doubles polished air flow 21180 where the RPM remains at 1000 and all MVVs are essentially the same design. Manifold polished air flow 21180 enjoys additional advantages of being steadier and more legato. Compared to the 58 liters per second difference between maximum and minimum flows of the four-MVV configuration, the difference between the maximum and minimum flows for an eight-MVV configuration is 21.7 liters per second, a decrease of about a factor of 2.7.

Still greater capacity, steadier polished air flow and a more pleasing legato may be achieved by adding additional sets of four MVVs. Three four-MVV sets may optimally run at about 60-degrees out of mutual rotational phase. Four four-MVV sets may be optimally run at about 45-degrees out of mutual rotational phase. N four-MVV sets may be optimally run at about (180/N)-degrees out of mutual rotational phase.

Referring to Fig. APS-01A, time varying polished air flow rates and sounds created by variations thereof by polisher 10124 may also be dampened and attenuated with one or more of the following:
1. The presence of any downstream components serves to dampen flow rate changes and accompanying sounds. Filter media are particularly effective.
   a. AMD 10125 (if present)
   b. Post-polisher 10126 (if present)
   c. AMD 10127 (if present)
2. Methods well known in the art of engine mufflers. An exemplary muffler design is illustrated in Fig. ICE-12. Muffler 21200 includes a perforated core conduit 21210 within a larger fiber lined 21220 sound absorbing chamber 21230. Sound absorbing chamber 21230 preferably includes elastomeric properties. When flow momentary increases, polished air exits perforated core conduit 21210 through perforations into sound absorbing chamber 21230. When flow momentarily decreases, polished air reenters perforated core conduit 21210 through its perforations.
3. An elastomeric component stretches on momentary polished air flow-pressure increases and contracts on momentary polished air flow-pressure decreases.
   a. An elastomeric membrane which is stretched when polished air flow rates momentary increase and contracts when polished air flow rates momentary decrease. Exemplary locations to include an elastomeric membrane include AMD 10125 (if present), post polisher 10126 (if present), and AMD 10127 (if present).
   b. An elastomeric tube which is deflected outward when flow rates momentary increase and return inward when flow rates momentary decrease. Exemplary locations to include a suitable elastomeric tube include but are not limited to flow paths 10135, 10136 (if present), 10137 (if present), and 10130. Fig. ICE-13 illustrates deformable elastomeric tube 21300. When air pressure inside and outside tube 21300 are equal, tube 21300 is in relaxed state 21310. When air pressure inside tube 1300 is greater than air pressure outside, tube 21300 deflects outwardly by a deflection amount 21320. When air pressure inside tube 21300 is less than air pressure outside, tube 21300 deflects inwardly by a deflection amount 21330.

### ICE—Independent Claims

A system and a method to reduce T1K and oxidizable T3K by isentropic compression and expansion of air.

A system and a method to reduce T1K and oxidizable T3K by isentropically induced oxidation.

A system and a method to reduce T1K by isentropically induced lysis.
ICE+N/4 MVV (integrated cycle)
ICE+>isochoric dwell
ICE+>isochoric dwell (within MVV)
ICE+>isochoric dwell (MVV=cam-guided rotary)
Above+MVV cooling
ICE+isochoric dwell (split-cycle w/oxidizer)
ICE+isochoric dwell (split-cycle w/oxidizer)+ZMV-MVV
ICE+isochoric dwell (split-cycle w/oxidizer)+even integral MVV pairs
ICE+isochoric dwell (split-cycle w/oxidizer)+catalyst
ICE+isochoric dwell (split-cycle w/oxidizer)+cooling (AC)
ICE+flow & sound dampening
ICE+flow & sound dampening: elastomeric
Above+MVV thermally integrated w/cooling
Above+MVV thermally isolated w/enhanced expander cooling
Above: ELF
Above+pre-polisher
Above+pre-polisher (HEPA)
Above+post polisher
Above+post polisher (adsorption media)
Above+purge/dilution

### Active Shadow (Trapdoor)

Target chamber polishers necessarily include orifices to allow introduction of designated air and removal of polished air. These same orifices allow photons out of a target chamber thus reducing photonic persistence within a target chamber. One embodiment of the present invention includes a moving member such as a “trapdoor” which can be actuated to temporarily close or cover an orifice or other non-reflective, or low-reflective surface. When trapdoors are closed, they seal air and photons in a target chamber; when trapdoors are open, air and photons communicate between the inside and outside of a target chamber. Inward facing surfaces of trapdoors are covered with reflective material.

Trapdoors may open to allow a batch of air into and/or out of a photon target chamber. When at least a portion of a volume has been replaced with feed air, trapdoors may be closed to boost photonic persistence in such a target chamber. The time between closing and reopening of such trapdoors defines an enhanced exposure phase. During an enhanced exposure phase, when trapdoor(s) to a target chamber is/are closed, feed air can be exhausted without further treatment, deadheaded, or a feed supply can be suspended. Alternatively, a plurality of photon target chambers can be provided, plumbed in parallel, and operated sequentially for nearly continuous treatment of feed air. Photon sources can be pulsed, on-off or high-low, when trapdoors are closed or open respectively. When trapdoors are opened a target chamber is less effective and pulsing photon sources off saves energy, reduces waste heat created by photon source(s), extends the life of photon source(s), and allows a photon source(s) to operate at a lower temperature. The latter feature improves performance and life of some photon sources.

Fig. PHO-04 provides an overlay of two cross-sectional views of an exemplary three-target-chambered trapdoor device to make cleaner air. Three cylindroid target chambers 0401, 0402, and 0403 with inner reflective surfaces 0471, 0472, and 0473 respectively are illustrated together in cross section with first cam wheel 0410 all housed within outer casing 0455. First cam wheel 0410 and a similar cam wheel (eclipsed in the illustration by first cam wheel 0410) rotate together about their common axis on axel 0415 in a clockwise direction as indicated by arrow 0417. A circumferential portion of cam wheel 0410 between thickening ramp 0412 and thinning ramp 0411 has a raised surface. Said raised surface protrudes perpendicular to the sectional view and toward target chambers 0401, 0402, and 0403. The raised surface ramps chamberward near 0412 and ramps counter-chamberward near 0411, such that a bearing and actuator that slides or rolls against the chamber-facing surface of cam wheel 0410, is urged chamberward at about 0412, and allowed to return anti-chamberward at about 0411. An actuator is biased anti-chamberward by a biasing force. A biasing force may be supplied by one or more of a mechanical spring, gravity, a deformable elastomer, or other similar means well known in the art. In Fig. PHO-04, target chamber 0403 has just opened, target chamber 0402 has just closed, and target chamber 0401 is closed.

Each target chamber 0401, 0402, and 0403 is illuminated by at least one photon source 0491, 0492 and 0493 each paired with orifice 0481, 0482 and 0483 respectively. Photons emitted from photon source 0491 project through orifice 0481 into target chamber 0401. Photons emitted from photon source 0492 project through orifice 0482 into target chamber 0402. Photons emitted from photon source 0493 project through orifice 0483 into target chamber 0403.

Figs. PHO-05A and PHO-05B provide two sectional elevation views of target chamber 0401 in Fig. PHO-04 renumbered in Figs. PHO-05A and PHO-05B to 0501. In Fig. PHO-05A target chamber 0501 is in an actuated state (i.e., closed) and in Fig. PHO-05B target chamber 0501 is in an unactuated state (i.e., open). Figs. PHO-05A and PHO-05B also provide cross-sectional elevation views of two portions of cam wheel 0410 in Fig. PHO-04 renumbered in Figs. PHO-05A and PHO-05B to 0510. A raised portion of cam wheel 0510 is designated as raised portion 0510r (i.e., the portion of cam wheel 0410 between thickening ramp 0412 and thinning ramp 0411). The terms raised and unraised are relative to a biasing force such as that provided by spring 0529c. In “actuated” illustration Fig. PHO-05A, raised portion 0510r of cam wheel 0510 is engaged with and displacing bearing or slider 0525 which in turn displaces actuator rod 0522 upward against biasing force of spring 0529c and stationary bulkhead 0530, which in turn urges trapdoor 0520 to close the bottom of chamber 0501. The upper surface 0521 of trap door 0520 is made of highly reflective material, generally of comparable reflectivity to inside surface 0571 of chamber 0501.

Fig. PHO-05C illustrates an exemplary design of stationary bulkhead 0535 designed to accommodate three target chambers such as target chambers 0401, 0402 and 0403 illustrated in Fig. PHO-04. Stationary bulkhead 0535 includes cutouts 0538 or perforations to allow air to easily pass through or around itself. Stationary bulkheads 0530 and 0535 may be identical. Bulkhead 0535 is fixedly attached within outer casing 0555 and includes a cutout to allow axel 0515 to pass therethrough. The purpose of stationary bulkhead 0535 is to provide an essentially immovable surface for spring 0529 (Figs. PHO-05A and PHO-05B) to act against. Stationary bulkhead 0535 includes at least one hole 0537 to accommodate actuator rods such as actuator rod 0522 (Figs. PHO-05A and PHO-05B).

Fig. PHO-05B shows an “unactuated” illustration of target chamber 0501 when a raised portion 0510r of cam wheel 0510 has rotated away from target chamber 0501. Absent a raised portion 0510r of cam wheel 0510, spring 0529u returns to a nominally uncompressed state against stationary bulkhead 0530 and opens trapdoor 0520. Target chamber 0501 in PHO-5B is the substantially enclosed volume enclosed by four surfaces: (1) the cylindrical interior surface wall 0571, (2) the static virtual wall at the interior end of orifice 0580 which accommodates photons from photon source 0590, (3) the dynamic virtual walls 0576 and 0574 depicted with dotted lines created upon the opening of trapdoors 0520 and 0570 respectively, and (4) the portion of the interior surfaces 0521 and 0569 of trapdoors 0520 and 0570 respectively circumscribed by virtual cylinder walls 0576 and 0574 respectively. Dynamic virtual walls 0576 and 0574 are illustrated with their largest dynamic areas achieved when trap doors 0520 and 0570 are maximumly open. Such virtual walls are dynamic virtual walls because their areas vary with the time varying position of active shadow components. In Fig. PHO-5A virtual walls 0576 and 0574 are not illustrated because their areas are each zero.

Target chamber 0501 is configured with a top and bottom to facilitate flow of air when both trapdoors are open. Airtight bulkhead 0550 prevents flow of air and entrained targets to take any path other than that afforded by open target chambers such as target chamber 0501 when trap door 0520 and trapdoor 0570 are open. In the previous discussion our attention was on the bottom portion, but the top portion works in the same way except that it is upside down relative to the bottom portion. Actuators, springs, and bulkheads provide the same functions except that they act in opposite directions. For example, cam wheel 0560 is about the same as cam wheel 0510 except that it is flipped over. Cam wheels 0510 and 0560 are linked by axel 0415 in Fig. PHO-04 and axel 0515 in Figs. PHO-05A and PHO-05B. Trap door 0570 is about the same as trap door 0520 except that it is inverted and its lower surface 0569 is reflective.

At least one photon source may be located inside chambers 0401, 0402, and 0403 or are more advantageously placed outside chambers 0401, 0402 and 0403 as illustrated in Fig. PHO-04 as photon sources 0491, 0492 and 0493 respectively. For photon sources located outside of target chambers at least one minimally sized orifice (e.g., orifices 0481, 0482 and 0483 correspond to photon sources 0491, 0492 and 0493 respectively) is preferably supplied for each target chamber-photon source pair and located through at least one of a target chamber wall illustrated 0481, 0482, 0483 of Fig. PHO-04) and a trapdoor (e.g., 0520 and 0570 of Fig. PHO-05A). Preferably such orifices may be located in a passive shadow.

Target chamber polishers illustrated in Figs. PHO-04, PHO-05A, and PHO-05B require air handling equipment and other elements of an APS as illustrated in Fig. APS-01A. For example, pre-polisher 10122 of Fig. APS-01A may include HEPA filters or other air cleaning means and is advantageously provided before air is introduced to target chambers 0401, 0402 and 0403 of Fig. PHO-04. Fans, ducting, and bulkheads are provided as well known in the art to direct designated air from 10134 of Fig. APS-01A to a first path 0598 of at least one trapdoor target chamber as illustrated in Fig. PHO-05B. Where two or more target chambers are employed as illustrated in Fig. PHO-04, and to enjoy balanced and generally continuous airflow, it is advantageous to operate trapdoors such that as a first chamber's trapdoors are closing, a next chamber's trapdoors are opening. After treatment polished air follows path 0599 illustrated in Fig. PHO-05B and is directed to flow path 10135 illustrated in Fig. APS-01A.

While actuating members illustrated in Figs. PHO-04, PHO-05A, and PHO-05B are cam driven, it is not a departure from the spirit of the present invention for actuation by other means including but not limited to electromagnetic, pneumatic, steam, hydraulic and hybrids of such actuating means.

An active shadow or trapdoor covers a less reflective or non-reflective surface with a surface with higher reflectivity. To appreciate an advantage of exemplary three-chamber trapdoor illustrated in Figs. PHO-04, PHO-05A, and PHO-05B, consider a nominally cylindrical target chamber with a diameter of about 40.6 cm, a length of about 81.3 cm, an average photonic path per reflection of about 45 cm, a Lambertian reflectance of 98% for all reflective surfaces including an inner surfaces of a trapdoor, inlet and outlet areas totaling about 10% of an interior surface area, and a single photon source measuring about 36 mm2 with a 50% reflectivity. Target chamber reflectance is about 97% with trapdoors closed and about 87.3% with trapdoors open. Photonic persistence with trapdoors closed is about 1.14 meters and about 0.2 meters with the trapdoors open or omitted. Active shadowing of air inlet and outlet orifices yields about a 5.7-fold improvement in photonic persistence.

Active shadowing may also be employed to cover sensor orifices where continuous sampling is not required.

While Figs. PHO-04, PHO-05A and PHO-05B illustrate a three-chamber cylindrical trapdoor APS target chamber with essentially flat trapdoors, substantial changes are possible without departing from the spirit of the present invention. Any natural number of chambers may be employed, and a chamber shape may be in whole or in part cylindrical, spherical, conic sections, toroidal, or other shapes that provide benefits to direct air flow paths and/or direct photon flux. Trapdoors may be flat, ellipsoid, paraboloid, hyperboloid, or any shape that improves photonic persistence. A trapdoor may be outside of a target chamber, inside a target chamber, configured to close flush with an inside surface of a target chamber, or a combination of these sealing modes.

An enhanced exposure time is determined by four variables:
1. An exposure chamber count,
2. a volume of each exposure chamber,
3. an air flow rate, and
4. a cam wheel angular speed greater than a threshold value, where the threshold value is determined by the first three variables.

As a non-limiting example consider a 3-count exposure chamber as illustrated in Figs. PHO-04, PHO-05A, and PHO-05B where each chamber is 10 volume units, and a flow rate is 25 volume units per second. A quantum of air entering such a target chamber will have an average residence time of about 1.2 seconds and about two-thirds of that time, or about 0.8 seconds, will be in an enhanced exposure phase as trap doors are closed. The balance of the target chamber residence time or 0.4 seconds is a valve-open time. The cam wheel angular speed should be greater than about 50 RPM so that less than 100% of the volume of each exposure chamber is flushed during a valve-open time. A slower cam wheel rotation speed allows more than 100% of a volume of the chamber to flush through an exposure chamber and hence that portion of supply air in excess of 100% of a volume of an exposure chamber would not be subject to an enhanced exposure phase. In this example a cam wheel angular speed of 60 RPM would allow about 83% of the volume to be flushed with each valve pair opening. Even greater angular speeds are possible but limited by energy efficiency, mechanical noise, or vibrational considerations inherent with higher angular speeds.

### Active Shadow (Trapdoor)—Independent Claim

A system and method to increase photonic persistence within a target chamber by moving at least one reflective surface to cover a LRS during an enhanced exposure phase and removing an at least one reflective surface at a conclusion of an enhanced exposure phase.
Active shadowing+>1 chamber parallel & sequential
Active shadowing+>1 chamber parallel & sequential+pulsed photon source (during enhanced exposure phase)
Above+cam wheel angular speed results in less than 100% volume flush during each valve-opening
Above+pre-polisher
Above+pre-polisher (HEPA)
Above+post polisher
Above+purge/dilution

### Passive Shadow

Photon sources, air inlets, air outlets, sensors and/or other low-reflective (including non-reflective) hardware include orifices between a PV interior and PV exterior or protrude into a PV chamber interior. In addition to low-reflective surfaces themselves, orifice and protrusion edges which necessarily surround all orifices and protrusions diffract incident rays which pass nearby. If a photon passes within its wavelength of an edge, it will be diffracted by Θ, where sin Θ=λ/d. There are two classes of shadows, namely active and passive. Active Shadows were described previously. This section describes five types of passive shadows, namely:
1. clock shadows,
2. deflection shadows,
3. virtual shadows,
4. attenuation shadows, and
5. Lambertian cosine shadows.

Equation 3 and Equation 4, collectively the shadow equations, provide an estimated target chamber reflectance, R_{TC}. The shadow equations provide estimates for any target chamber. Target chambers may include no shadows (i.e., Sn is equal to 100% for all values of n), passive shadows, active shadows, and a combination of active and passive shadows. Where active shadows are employed, Equation 3 may be solved for open trapdoors, closed trapdoors, or trapdoors that are transitioning between open and closed.

$${R}_{TC} = \{ [ Σ {A}_{u , m} + Σ ( {A}_{s , n} \cdot ( 1 - {S}_{n} ) ) ] \cdot {R}_{u} + Σ ( {A}_{s , n} \cdot {R}_{s , n} \cdot {S}_{n} ) \} / ( Σ {A}_{u , m} + Σ A s , n ) \qquad ( 3 )$$

$${R}_{u} = Σ ( {A}_{u , m} \cdot {R}_{u , m} ) / Σ {A}_{u , m} \qquad ( 4 )$$

R_{TC} is a target chamber reflectance, A_{u,m} is a discrete unshadowed area (m) within a target chamber which includes an approximately homogenous surface reflectance of R_{u,m}. A_{s,n} is a discrete shadowed area (n) within a target chamber which includes an approximately homogenous reflectance of R_{u,n} and an approximately homogenous shadow of Sn. All discrete areas, m and n, sum to the total area of a target chamber, and summations (i.e., Σ) include all discrete areas from 1 to m and from 1 to n, as appropriate to respective subscripts m and n. Ru is an area weighted average of reflectance within the unshadowed portion of a target chamber and is defined by Equation 4.

The first term in the numerator of Equation 3 represents the total unshadowed area of the target chamber. The second term represents the portion of photonic flux reflected into the unshadowed volume of the target chamber by active or passive shadowing features. The sum of the first two terms is multiplied by the area weighted average unshadowed reflectance, Ru. The third term summed with the first and second terms represents those photons that stray into shadowed portions of a target chamber. The denominator is the sum of the unshadowed and shadowed areas of the target chamber.

Discrete areas, Aₘ and Aₙ, may be of any magnitude including differential areas. Where differential areas are appropriate, summations become analogous integrals.

Together equations (3) and (4) allow the evaluation of a performance advantage afforded by employing shadowing features in a target chamber. Unity less a ratio of a target chamber reflectance R_{TC} to the greatest of the set R_{u,m}, where m=1 to M and where M is the last discrete unshadowed area, of unshadowed target chamber reflectance provides a variance from an ideal case. An ideal case is where a target chamber reflectance R_{TC} is equal to the greatest surface reflectance. R_{TC} is equal to R_{u,m}, and the variance from ideal is zero. For any target chamber design employing a geometry and materials with associated reflectance values a hypothetical unshadowed case can be calculated by setting all shadow values to 100%, i.e., no shadow. The advantages afforded by employing at least one shadow may be gauged by comparing the relative improvement in the variance from ideal. The relative improvement is the ratio of an absolute improvement in the variance from ideal to the hypothetical variance from ideal. The absolute variance from ideal is the difference between a shadowed variance from ideal and the hypothetical (unshadowed) variance from ideal.

Referring to Fig. PHO-02 APS target chamber performance 10230 measured on log-axis 10220 is a function of target chamber reflectance (R_{TC}) and any improvement in the latter provides a benefit to the former. The benefits are greater as the target chamber reflectance exceeds 80%, are even greater as the target chamber reflectance exceeds 90%, and are even greater as the target chamber reflectance exceeds 95%.

The application of shadows which yield even small improvements to target chamber reflectance may yield substantial improvements to target chamber performance. To illustrate these relationships, consider an example of a nominally cylindrical target chamber such as that depicted in Fig. PHO-05B that has an inside diameter 0577 of about 40.6 cm and a length 0578 of about 81.3 cm. Proximate each end of the cylinder are disks 0520 and 0570 with similar nominal diameters such that each disk may be sealingly mated with the cylinder or may be moved away from the cylinder end to create a clearance 0579 of about 10.2 cm to allow air to pass through the cylinder interior. The interior facing walls of the cylinder and the pair of disks are made from a material that has a reflectance of 97%. The target chamber is illuminated with photon source 0590 through an aperture 0580 with a reflectance of 25% and is situated within and flush inside the cylinder with an area of about 100 cm². Solving equations (3) and (4) when both disks are axially displaced from the cylinder ends by clearance 0579 of about 10.2 cm the hypothetical shadowless target chamber reflectance, R_{TC}, is about 80.37%, the ratio of this value to the maximum 97% material reflectance is about 82.9% and represents about a 17.1% deviation from ideal.

Consider a first example where a passive shadow feature (S=50%) casts a shadow upon the photon source, the R_{TC} improves to about 80.55% and the ratio of this value to the 97% material reflectance is about 83.0% or about a 17.0% variation from ideal. The relative variance improvement over the hypothetical unshadowed case is 1.1% and commensurately yields about a 1.1% increase in photonic persistence.

Consider a second example where an active shadow (S=5%) is employed by moving both disks to sealingly engage with the cylinder ends as in Fig. PHO-05A, the R_{TC} improves to about 96.44% and the ratio of this value to the 97% material reflectance is about 99.4% or about a 0.6% variation from ideal. The relative variance improvement over the hypothetical unshadowed case is 96.7% and yields about a 400% increase in photonic persistence.

Consider now a third example which combines the passive shadow of the first example with the active shadow of the second example, the R_{TC} improves to about 96.72% and the ratio of this value to the 97% material reflectance is about 99.7% or about a 0.3% variation from ideal. The relative variance improvement over the hypothetical unshadowed case is 98.3% and yields about a 460% increase in photonic persistence. In this third case, the improvement resulting from the addition of the passive shadow feature to the photon source was amplified by the steep slope illustrated by Fig. PHO-02 where the R_{TC} is greater than 90%. In example 1, the addition of the passive feature yielded a 1.1% increase in photonic persistence and in example 3, the addition of the same passive feature, but in combination with the active feature of example 2 yielded about an 11% improvement in photonic persistence.

As illustrated in the examples above, each shadow class and passive shadow type may be used independently or in combination.

### Clock Shadow

Photon sources, air inlets, air outlets, sensors and/or other low-reflective (including non-reflective) hardware may be in a shadow of inwardly angled protrusions on an inside surface of a cylindroid or prism target chamber configured as a photon vortex. Inwardly angled protrusions resemble sawteeth. The face of at least one sawtooth nominally parallel to a cylindroid or prism radius lies either clockwise or counterclockwise to an axis. Where a plurality of sawteeth is employed, all face a consistent clock direction. In the interest of brevity, the counterclockwise case will be described hereafter, but a clockwise sawtooth is equally as effective. It is not even necessary to define an axis perspective to determine clockwise and counterclockwise as the two are equivalent if all such sawteeth (which may be a single sawtooth in a full 360-degree cylindroid or prism or may be a plurality of sawteeth) are oriented to a same clock direction. A non-limiting example section of a positive 4-toothed sawtooth cylindroid target chamber polisher is illustrated in Fig. PHO-06. Positive sawteeth 0621, 0622, 0623, and 0624 are created by an inward protrusion to an otherwise generally cylindrical or prism-like inside reflection surface. A second non-limiting example of a passive clock shadow feature, a single negative sawtooth target chamber polisher, is illustrated in Fig. PHO-08. A third non-limiting example of a passive clock shadow target chamber polisher is illustrated in Fig. PHO-09.

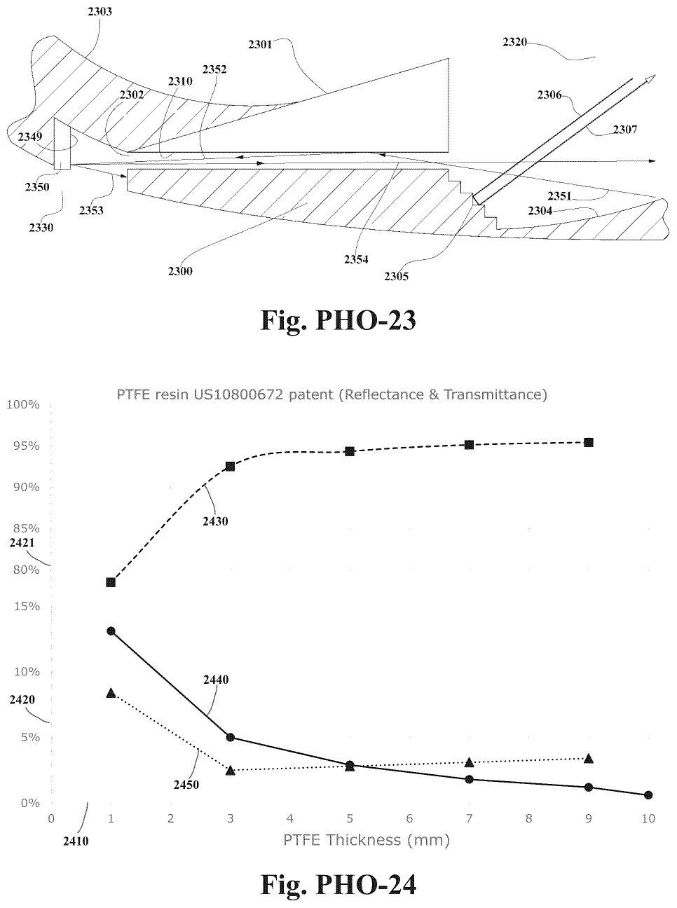

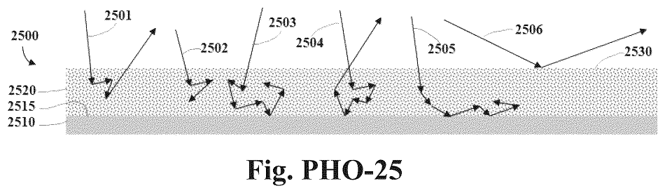

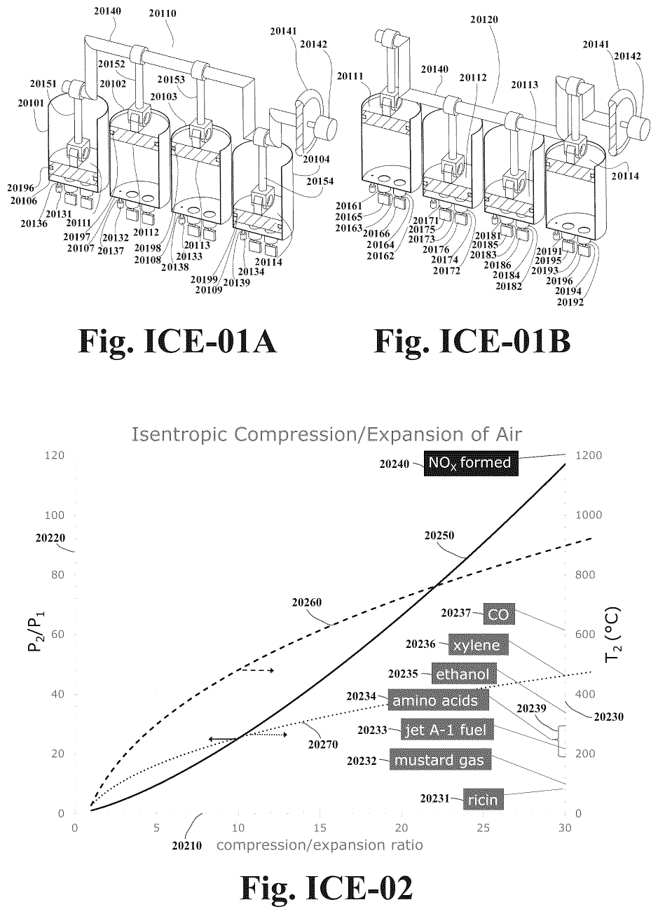

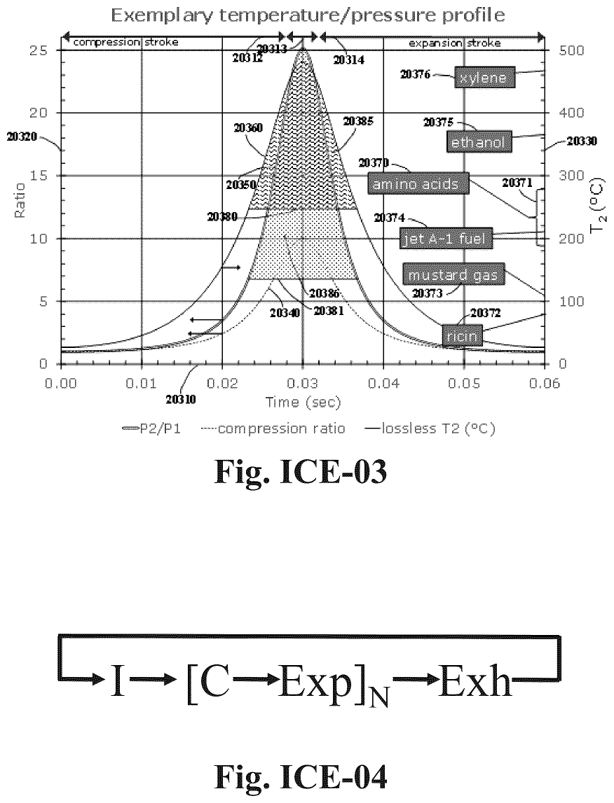

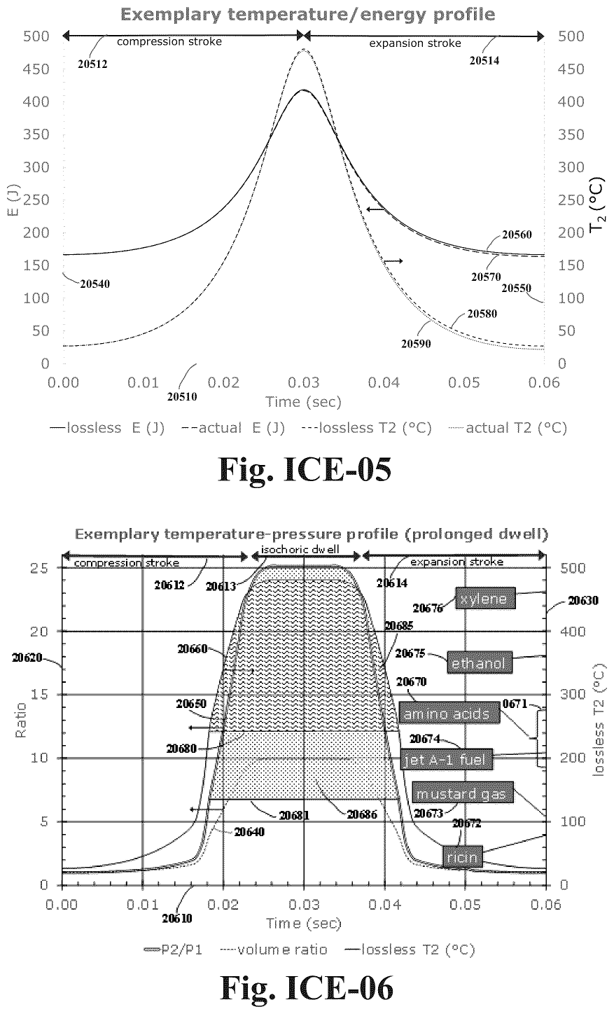

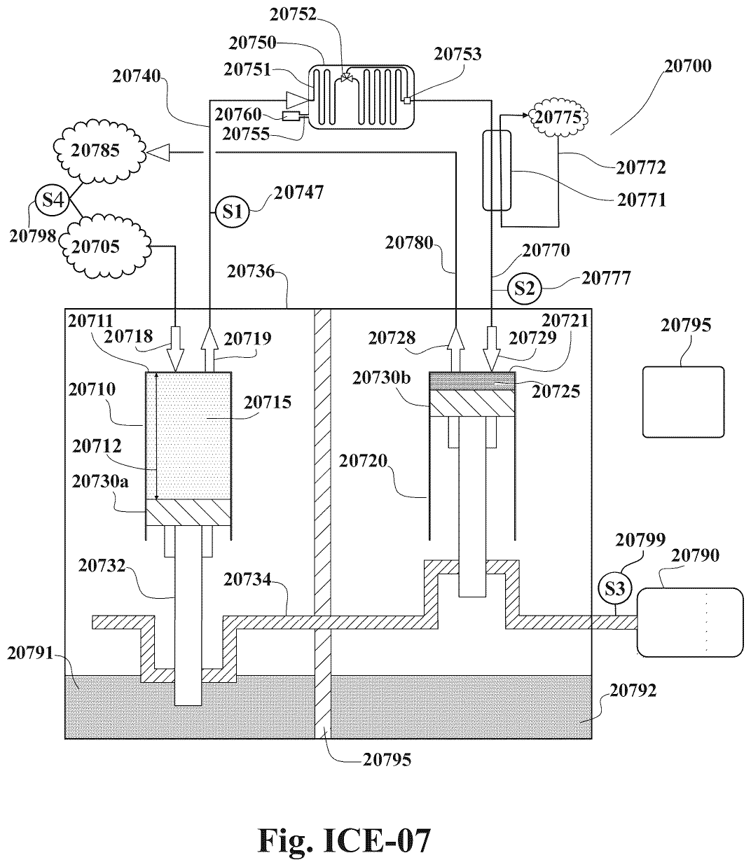

Referring to Fig. PHO-06, an initial photonic trajectory is approximately tangential to an inner surface of sawtooth cylindroid 0610 in a counterclockwise direction. Initial photonic trajectory can be projected by a laser or a focusing mechanism may be utilized to direct photons from a non-laser source into a cone of acceptable trajectories approximately perpendicular to a radius of a generally cylindrical cross-section near the saw-tooth. An exemplary focusing mechanism is a tailored edge-ray reflector known in the art of non-imaging optics. See for example Fig. 10 of WO2022046874A1.

Angle 0630 of a protrusion of sawtooth 0621 from the inner circumference of cylindroid 0610 are slight such that photons striking sawtooth protrusions are deflected only slightly more inwardly than would have occurred had a cylindroid not included a sawtooth. “Slightly more inwardly” defines an angle of incidence greater than or equal to an angle that is a critical angle of incidence, below which specular reflectivity is unduly compromised.

When one or more sawteeth are utilized to introduce fluid into a target chamber, said fluid will possess counterclockwise angular momentum relative to an axis of a roughly cylindrical target chamber. That angular momentum will ensure that a standard deviation of average residence time is small and average residence time is nominally uniform. Those skilled in the art of fluid dynamics will recognize that withdrawal of fluid from similar (counterclockwise) sawteeth elsewhere in a target chamber will not be facilitated by the required geometry and an angular momentum of a fluid. This is not a substantial issue as fluid flow efficiency is not a primary consideration.

A third independent parameter that can affect reflectivity is polarization of incident light. To this end, polarized laser photons may be utilized as a photon source to optimize reflectivity.

Referring to Fig. PHO-07 (an extension of Fig. PHO-06), if an angle of incidence is controlled within a band from just slightly less than 90° to above a specified value (e.g., about) 85° photons are confined for many reflections within a ring near the outside of a specular reflective inside wall 0720 of cylindroid 0700. A central portion of cylindroid 0700 falls in virtual shadow 0795. In this example virtual shadow 0795 is augmented by an attenuation shadow as many reflections occur before a photon crosses virtual wall 0790. Within virtual shadow 0795 air handling equipment, sensors and other equipment can be placed with muted illumination and hence muted photonic loss. A photon vortex exists in an annular region between inside wall 0720 of sawtooth cylinder 0700 and virtual wall 0790. Placing equipment within virtual wall 0790 has an additional benefit of displacing air which otherwise would not be in a photon vortex and hence not being treated with the same intensity. In illustrations Fig. PHO-06 and Fig. PHO-07 a photon strikes a reflecting inner wall 8 times and forms an irregular approximate nonagon with an average angle between an incoming photonic vector and an outgoing photonic vector of about 140°. Thus, the AOI and angle of reflection are both about 70° (i.e., 20° from grazing). Larger initial AOIs yield virtual walls with greater diameters expressed as a percentage of a nominal inside diameter of a sawtooth cylinder. The converse is equally true. Decreasing the diameter of a sawtooth cylinder while keeping the AOI constant increases the number of reflections in a single orbit and decreases the distance between reflections. Again, the converse is true. The geometry can be tailored using the AOI and a diameter to prescribe the geometry of a photon vortex, and more importantly, tailor an AOI to optimize a trade-off between specular reflectivity, which is a function of AOI, and a length of a flight path between reflections.

In the sawtooth cylindroid target chamber polisher described above, the introduction of one or more sawteeth necessarily interferes with the cylindrical geometry and hence forces deflections of photons inwardly from narrow cyclonic bands. The virtual wall 0790 greatly reduces the need for a plurality of sawteeth and/or the saw-tooth size, because air inlet(s), air outlet(s), and any instrumentation can be housed within a central virtual shadow. Only one or more photon source(s) need to be in a sawtooth shadow. The sawtooth need only be as large in an axial direction as is necessary to cast a shadow on minimally sized photon source(s).

Fig. PHO-08 provides a partial section view 0810 of a second version of a sawtooth cylindroid target chamber polisher. This negative sawtooth 0820 is well suited for near-tangential introduction of photons 0840 from a photon source 0830. Negative sawtooth 0820 minimizes inward deflection of photons for very small orifices designed to communicate between an inside and an outside of a cylindroid. Photons 0840 may be introduced in very narrow beams (e.g., laser beams, partially collimated beams, specular light pipes, and non-imaging optics such as a tailored edge-ray concentrator) allowing communication orifices to be minimally sized. Positive and negative sawtooth features may be combined. Orifices may be cylindrical or may be other shapes such as rectangular. A negative sawtooth includes an essentially flat portion 0850 which deflects near grazing counterclockwise photons inwardly relative to a hypothetical reflected trajectory absent negative sawtooth 0820. This causes those photons to decrease the AOI of subsequent reflections.

Certain optical manufacturing processes deposit reflective coatings on substrates such as ceramic, glass, metal, or plastic, utilizing process including but not limited to ion-beam deposition (IBD), ion-assisted deposition (IAD), and ion-plating. Another embodiment facilitates these manufacturing processes by utilizing a plurality of partial cylindroids. Fig. PHO-09 provides a section view of an exemplary embodiment of sawtooth cylindroid target chamber polisher 0910 including two partial cylindroids—a left half cylindroid 0911 and a right half cylindroid 0912. Each partial cylindroid or sub-cylindroid includes a unique centerline-segment perpendicular to a section view. The left half cylindroid 0911 has a centerline 0921 and the right half cylindroid 0912 has a centerline 0922. The centroid 0929 of the two half cylindroids is located at a point equidistant from centerlines 0921 and 0922. In Fig. PHO-09 the centerline-segment is defined by an intersection of all perpendicular radial lines from the inside surface of each sub-cylindroid 0911 and 0912. Each centerline-segment is generally parallel to each of one or more other centerline-segment(s) and offset by a thickness 0930 of an adjacent sub-cylindroid(s) plus a clearance 0940 just large enough to accommodate the introduction of photons, fluid communication between the interior and exterior of the cylinder, and/or any other communication between an interior and exterior. The thickness offset 0930 plus a clearance 0940 define a step 0970 illustrated at the 6 o'clock position and step 0971 at the 12 o'clock position.

Photons 0950 introduced by photon source 0990 project through a clearance distance 0940 at step 0970 and repeatedly undergo specular reflection with an angle of reflection 0981 and identical AOI. Subsequent reflections are illustrated by ray paths 0951, 0952, 0953, 0954, 0955, 0956, 0957 and 0958. After ray 0958 passes step 0971 an AOI 0982 of subsequent reflections increases modestly, proportionate in part to the ratio of step 0971 and a circumcircle of previous ray paths. Subsequent ray paths 0959, 0960, 0961, and 0962 combine with ray path 0951, 0952, 0953, 0954, 0955, 0956, 0957 and 0958 to yield a photon vortex—a counterclockwise path generally around centroid 0929. Each time a ray path passes steps 0970 and 0971 the AOI of subsequent reflections increases slightly. Ray paths converge inwardly each time a step is crossed and converge asymptotically toward an AOI of 0°. Because each reflection involves some attenuation of an incident ray, photon flux is greatest near inner reflective surfaces of sawtooth cylindroid 0910 and diminishes nearer centroid 0929. The inner portion of sawtooth cylindroid 0910 thus enjoys a virtual shadow and an attenuation shadow. This passive shadow is well suited for the introduction of air to be polished from flow path 10134 illustrated in Fig. APS-01A and a supply of polished air into flow path 10135 illustrated in Fig. APS-01A.

While PHO-09 depicts two equally sized half-cylindroids 0911 and 0912 and hence two equal steps 0970 and 0971, other configurations are possible and enjoy certain advantages. For example, two half-cylindroids with diameters differing by a single step may be joined flush at the 12 o'clock position such that a single step is at the 6 o'clock position. This configuration requires a single step rather than the two steps illustrated in Fig. PHO-09 and hence enjoys an improved virtual shadow (i.e., a lower photon flux) near its centroid.

Nominal cylindroid ends may be flat mirrors. Cylindroid walls are preferentially curved inwardly as described in Photon Vortex or by other methods including those described in WO2022046874A1 and GB2593827A or others known in the art to minimize photonic interaction at cylindroid ends.

In another embodiment a polygonal cross section can be substituted for generally circular cross sections previously described. Fig. PHO-10A illustrates a hexagonal cross section of a prismatic photon vortex 1000 and Table 1 lists key parameters for hexagonal and other polygonal prisms together with some key values illustrated in Fig. PHO-10A. While a single photon source 1010 is illustrated, more photon sources may be included in other sections and/or from other vertexes.

Fig. PHO-10A provides a section view of a hexagonal prismatic photon vortex target chamber polisher 1000 with sidelength equal to L_{S} and diameter of 2L_{S}. Each of the six faces (F1-F6) include a specular reflective inside surface. Each face may overlap inwardly from its adjacent counterclockwise face such that photons incident from a counterclockwise direction do not encounter an acute edge. A larger but still diminutive overlap is illustrated between faces F6 and F1 to accommodate at least one photon source 1010 emitting a cone of photons. The cone angle 1015 is about 25°. One of six vertex diagonals 1003 connects two non-adjacent vertexes (V5 and V1) of adjacent faces (F5 and F6). The vertex diagonal 1003 length is about 1.732 L_{S} and together with two sidelengths F5 and F6 form an isosceles triangle with one obtuse vertex angle of 120° at V6 and two acute vertex angles of 30° at V5 and V1. A vertex to diagonal line segment 1004 is equal to about 0.5 L_{S}. The photon cone emanating from photon source 1010 is delineated with two edge-rays. The edge-ray 1020 is approximately parallel to a bottom horizontal reflective face F1 and edge-ray 1030 diverges away from face F1 at an angle of about 25°. The two edge-rays 1020 and 1030 are reflected around the six faces as edge rays 1021, 1022, 1023, 1024, 1025, 1026, and 1031, 1032, 1033, 1034, 1035, 1036 respectively. The shaded area 1040 between those edge-rays (and a corresponding volume in three-dimensions) highlights photon rich zone 1040 or a photon vortex 1040. Seven unshaded areas within prismatic photon vortex 1000 are shadowed. Six virtual shadows 1051, 1052, 1053, 1054, 1055 and 1056 are located near vertexes V1 through V6 respectively. Those shadows serve to minimize diffraction at mirror edges. The shadow 1051 at V1 is virtual as are those at V2 through V5, but shadow 1051 also includes a clock shadow to prevent the photon source 1010 from absorbing photons. Seventh shadow 1057 is a photon-sparse nominally hexagonal prism-shape near a central axis of prismatic photon vortex 1000.

To maintain a photon vortex, keeping a substantial portion of photon trajectories in a consistent clock direction (counter-clockwise in Fig. PHO-10A), least-parallel edge-ray 1030 of photon source 1010 must strike an obtuse face. In example hexagonal photon vortex 1000, there is a single face, F2, that meets this requirement. The acute vertex angle for a hexagon is about 30° and hence least-parallel edge-ray 1030 is preferably less than 30°. A greater angle may be utilized, but a portion of a cone of photons greater than 30° will impinge upon shadowed areas and hence lessen the effectiveness of a photon vortex. For polygonal prisms with 8 or more faces the photon source cone may strike more than a single face without striking an acute face. The number of such “adjacent non-acute faces” are tabulated in Table 1. For example, two adjacent faces on an octagon do not create an acute angle from a third, adjacent face. As the number of faces becomes greater the number of adjacent, non-acute faces also increases. For example, an 80-face octacontagon has 20 adjacent non-acute faces.

In Fig. PHO-10A photon source 1010 is directed such that the most parallel edge-ray 1020 is approximately parallel to face F1. The photon source 1010 can also be aimed downward so that at least a portion of the emitted photons reflect from F1 before reflecting onto F2. For example, if cone angle 1055 of photon source 1010 was 50° and photon source 1010 was rotated clockwise by about 25°, least-parallel edge-ray 1030 would be in about the same location, but a new ray 1060 would be sloping 25° downward from the horizontal at an angle of incidence of about 65° to F1 from photon source 1010. Edge-ray 1060 would almost immediately strike face F1 and reflect upward as ray 1061 at about a positive 25° (a 65° angle of reflection) from F1, about parallel and below least-parallel edge-ray 1030. Aiming a photon source in this way, the cone angle can be “folded” to reduce an effective cone angle by any fraction up to one-half. Cone angle folding allows the use of wider photon source concentrating optics and/or to create a narrower photon rich zone and a larger virtual shadow near an exemplary hexagon centroid.

Even where a photon source is a laser, photons diverge from a single vector in two dimensions. A partially collimated beam diverges even more than a collimated laser. A first dimension of this divergence is illustrated in Fig. PHO-10A and is described in the narrative above for a photon source with a 25° divergence. A second dimension of divergence falls in a plane perpendicular to the section view of Fig. PHO-10A and axis 1099 of prismatic photon vortex 1000 of Fig. PHO-10B. Referring to Fig. PHO-10B, a pair of edge rays delineate this second dimension of a conical dispersion from photon source 1010. Relative to photon source 1010 a first edge ray 1070 and a second edge ray 1080 delineate a cone of photons. Axial conical angle 1090 between first edge ray 1070 and second edge ray 1080 is illustrated in PHO-10B and may be about the same as conical angle 1015 in the section plane of Fig. PHO-10A, it may be slightly different, or it may be very different. At least one photon source 1010 may be anywhere along a length of prismatic polygon 1000. In PHO-10B photon source 1010 is illustrated about midway between a first end-mirror 1091 and a second end-mirror 1092 of prismatic photon vortex 1000. As illustrated, photon source 1010 may be directed so that about half of its emitted photons travel toward first end-mirror 1091 and about half of its emitted photons travel toward second end-mirror 1092. A photon source may be biased toward either end-mirror such that any fraction of emitted photons can be directed toward first end-mirror 1091 and, of course, the complementary fraction directed toward second end-mirror 1092.

End-mirror 1091 and end-mirror 1092 may be essentially the same or may be different. Inner reflective surface 1093 of end-mirror 1091 and inner reflective surface 1094 of end-mirror 1092 reflect incident photons back into photon vortex 1040. End mirror 1091 includes orifice 1095 to allow fluid communication between flow path 10134 of Fig. APS-01A carrying designated air and prismatic photon vortex 1000. End mirror 1092 includes an orifice 1096 to allow fluid communication between flow path 10135 of Fig. APS-01A and prismatic photon vortex 1000 to carry polished air. Orifice 1095 and orifice 1096 of target chamber polisher 1000 are positioned within virtual shadow 1057.

When an incident photon has any non-perpendicular axial angle, and if it is not attenuated first, it will eventually intercept an end-mirror of photon vortex 1000. Each such end encounter is a non-productive reflection. A non-productive reflection is a reflection that shortens the flight path length of a photon without advancing a photon's path in a clock direction (counterclockwise in Fig. PHO-10A) of vortex 1040. To minimize non-productive reflections from an end-mirror, axial conical angles close to perpendicular to any line parallel to axis 1099 are most preferred. An axial conical angle is preferably greater than 85°, greater than 77.5°, greater than 45°, greater than 30°, greater than 15°, and greater than 0°. For example, in Fig. PHO-10B edge ray 1070 is incident at axial angle 1079 of about 77.5° upon face F2 and reflects as ray 1071. Ray 1071 then becomes incident upon face F3 and reflects as ray 1072. Ray 1072 then becomes incident upon face F4 and reflects as ray 1073. Ray 1073 then non-productively reflects from hexagonal end mirror 1091 as ray 1074. Ray 1074 then becomes incident upon face F5 and reflects as ray 1075. Ray 1075 continues counterclockwise (i.e., F6→F1→F2→F3→F4→F5→F6→F1 . . . ) toward end mirror 1092 and where it is next incident upon face F6, then face F1, then then face F2 and continues in this manner until it is incident in a second non-productive reflection at second end mirror 1092. This process repeats indefinitely until the exemplary ray has been attenuated to zero photons.

The discussion above focuses on specular reflection, but some concepts described have applications with diffuse reflection. As introduced in Angle of Incidence (Diffuse Reflection) the three-dimensional spatial distribution of reflected photons from diffuse reflectors is not uniform for a given angle of incidence and the angle of incidence itself is non-uniform. Such non-uniformities can be exploited with off-the-shelf polymer designs or polymers may be modified to enhance non-uniformity. As a non-limiting example, suppose that glancing angles of reflection of 1-3 degrees are quite rare and a disproportionately larger number of reflections occur from 3-90 degrees. This is the case for Lambertian reflectors and is defined by Lambert's cosine law. The sawtooth cylinder illustrated in Fig. PHO-06 would benefit from that case as fewer photons would be directed at a less reflective, nominally radial faces of sawteeth, particularly if such teeth deviate inwardly less than 3 degrees. This idea will be advanced further in Photonotron.

The sectional, two-dimensional views of Fig. PHO-06 through Fig. PHO-10A involve at least one photon source and an orifice through which photons are delivered from outside a photon vortex into a photon vortex. These orifices may lie within passive shadows. In each case it is preferred that one or more orifices are of a minimal size required to deliver photons from an external photon source to a photon vortex. A portion of rays incident upon orifice edges are diffracted onto paths slightly askew to an orifice edge. For example, if a photon vortex axis was about 200 mm long and a nominally circular orifice of 1 mm delivered photons to the vortex, each photon would have less than a 0.5% chance of interacting with the orifice or the orifice edge on a single 360° cycle if no shadowing measures were employed. The probability of edge interaction and diffraction is thus minimized by minimizing orifice size.

### Deflection Shadow

Deflection shadows are preferably created by locating an orifice at an inwardly curved (i.e., convex) periphery of a photon vortex or a PRC mirror. The inward curvature at a PV periphery (i.e., axis ends) urges photons toward a middle portion (i.e., nearer an axis midpoint) of a PV such that photonic density is greatest distal from both peripheries and least at a photon's introductory peripheral orifice location. A second preferred deflection shadow is created away from the periphery and nearer a central portion of a PV. At least one orifice is located near a maximum inward convex bulge around the periphery or circumference of the PV's reflective interior. The convex bulge urges photons away from itself thus splitting a photon source into a first portion and a second portion. Each portion would generally remain between a central bulge and a first end and second end respectively.

### Virtual Shadow

In contrast to clock shadows and deflection shadows which require structural features to create a shadow, a virtual shadow is created by judicious aiming of collimated or partially collimated photon sources such that subsequent reflections of said beam are not incident near or upon a shadowed feature. Figs. PHO-11A and PHO-11B provide a non-limiting example of a virtual shadow 1190. Table 2 provides a compilation of whole number beam angles in a photon vortex with a circular cross section. Selected values (columns) for selected whole number beam angles (rows) for circular cross sections of a PV chamber with a diameter, D. Beam angle is an angle in degrees of a ray introduced through an orifice from outside of a PV into a PV where the angle is measured from a tangent to a circular cross section at the inside surface of an orifice. The subtended angle is the angle of a vertex at the circle center of a triangle whose base is a chord, and the chord is a beam. Chord is a chord length expressed as a fraction of a circle diameter, D. Virtual shadow diameter is expressed as a fraction of the PV chamber inner diameter, is less than or equal to the inside diameter of a PV chamber, shares the same center with a PV cross section, and is in a virtual shadow. The Vertexes column provides the number of reflections experienced by a photon as it reflects around a single trip around an inside circumference. Figs. PHO-11A and PHO-11B and Table 2 are not limiting to the scope of the present invention as real number beam angles, ellipsoidal cross-section PVs, and prismatic PVs are additional examples of workable geometries.

Figs. PHO-11A and PHO-11B provide two section views of PV target chamber polisher 1100. Fig. PHO-11A illustrates at least one photon source 1110. The photon source 1110 introduces a collimated or partially collimated ray, 1111, through an orifice 1180 into PV 1100 at about 61° from a tangent to a circular cross section. Ray 1111 reflects from a first vertex to become ray 1112. Ray 1112 reflects from a second vertex to become ray 1113. Ray 1113 reflects from a third vertex about 6° counterclockwise from orifice 1180 to become ray 1114. Orifice 1180 is in a virtual shadow as ray 1113 does not approach orifice 1180 within a photon width. The right view shows ray 1111 through ray 1156, with only ray 1156 being explicitly identified. Like ray 1113, ray 1156 is not incident upon orifice 1180 and hence orifice 1180 remains in a virtual shadow for at least 56 reflections. As ray 1156 continues to propagate it may eventually fall incident on orifice 1180. Careful angle selection and scores of reflections creates an attenuation shadow for orifice 1180 in addition to the previously described virtual shadow.

The area near the center of PV target chamber polisher 1100 and inside a nominal circle 1195 is virtual shadow 1190. This passive shadow is well suited for the introduction of designated air to be polished from flow path 10134 illustrated in Fig. APS-01A and a supply of polished air into flow path 10135 illustrated in Fig. APS-01A.

Fig. PHO-12 provides a graphical view of values in Table 2 and illustrates the trade-off between chord length and virtual shadow diameter. The beam angle from 0° to 90° is represented by X-axis 1210. Chord length 1230 and virtual shadow diameter 1240 are expressed as a fraction of a cylindrical diameter on Y-axis 1220. Photonic persistence determines target chamber performance and all else being equal an increase in photonic path length leads directly to a proportional improvement in photonic persistence. As illustrated in Fig. PHO-12 as beam angle increases from 0° (perpendicular) to 90° (glancing) the chord length decreases from one diameter to zero diameters with the sine of the AOI. Thus, it is advantageous for photonic path length to decrease beam angle.

Centered on a PV target chamber polisher axis a virtual shadow provides an ideal location for air handling equipment to introduce designated air to be polished from flow path 10134 illustrated in Fig. APS-01A and polished air into flow path 10135 illustrated in Fig. APS-01A. Virtual shadow diameter is impractically small at the right side of Fig. PHO-12 to move substantial quantities of air to be polished. A third consideration is reflectivity of the interior of a PV target chamber. Reflectivity is not constant for all AOIs; judicious compromises are required to optimize a PV target chamber polisher. In a preferred embodiment of the present invention (1) air ducts are made as small as practical to minimize a virtual shadow diameter, (2) Table 2 and Fig. PHO-12 are consulted to determine the greatest possible beam angle, (3) a slightly smaller beam angle is chosen to take advantage of virtual shadows and attenuation shadows, and (4) a specular reflective surface is optimized for the chosen maximum beam angle. A collimated beam is preferred so that all photons follow a single defined path. Partially collimated beams are also in the scope of the present invention.

Fig. PHO-13 illustrates the case of PV target chamber polisher 1300 where photon source 1310 and its associated optics do not produce a collimated beam. Edge rays 1302 and 1303 depict a diverging beam from photon source 1310 and orifice 1301. Preferably a photon source is judiciously aimed so that neither 1302, 1303, or any rays between 1302 and 1303, will be incident upon an orifice upon their third reflection, depicted as edge rays 1306 and 1307. Subsequent reflections will proceed analogously to those illustrated in Figs. PHO-11A and PHO-11B. After about 52 to 56 reflections a portion of the beam will be incident upon the orifice. The total photon loss from this interaction will be mitigated by a ratio of surface area of an orifice relative to surface area of a divergent beam and by an attenuation shadow, described in the next section. Preferably photonic loss can be further mitigated with a deflection shadow.

The area near the center of PV target chamber polisher 1100 and inside a nominal circle 1195 is virtual shadow 1190. This passive shadow is well suited for the introduction of designated air to be polished from flow path 10134 illustrated in Fig. APS-01A and a supply of polished air into flow path 10135 illustrated in Fig. APS-01A.

### Attenuation Shadow

An attenuation shadow is created by aiming photon beams or rays such that a large number of reflections occur before a photon might interact with an orifice or other non-reflective feature. For illustration purposes and referring to Fig. PHO-11B, suppose ray 1111 is comprised of 100 photons and the reflectivity of PV 1100 is about 99% at the AOI illustrated. With each photon-reflector interaction there is about a 99% probability that a photon will reflect and about a 1% probability that a photon will be absorbed. By the time that the 100 original photons undergo 56 reflections after ray 1156 and are incident near a photon source, about half will have attenuated. That is, ray 1156 includes about 58 of the original photons of ray 1111. Another 58 reflections, around PV 1100 puts those remaining photons still closer to but not incident upon orifice 1180 and about 31 photons remain. A ray after about 116 reflections is about 69% attenuated compared to ray 1111. Orifice 1180 is in an attenuation shadow, where S is about 31% or less.

In the discussion of passive shadowing and the virtual shadows illustrated in Fig. PHO-07 and Fig. PHO-10A, specular reflection directs photons on a geometrically influenced pre-determined path. In Fig. PHO-07 the annular space between the inside diameter of the target chamber and the event horizon is a photon rich zone. To encourage collisions between photons and targets air handling equipment must deliver air together with entrained targets into this photon rich zone. Photon rich zones can be shaped by the arrangement of mirrors and surfaces, the initial aiming of photon light source(s), and the uniformity or lack of uniformity of wave lengths and polarities of the photon source(s).

### Lambertian Cosine Shadow

Fig. PHO-14 illustrates Lambert's cosine law as 17 arbitrarily sized wedges of equal angles de measuring about 10.6° (i.e., the quotient of 180° and 17 wedges) of circle 1400. Wedge 1401 and wedge 1417 are at near glancing angles to Lambertian horizontal surface 1450 and are the smallest of the 17 wedges. For Lambertian surface 1450, a number of photons per unit time emitted into each wedge is proportional to each wedge area. A small fraction of photons reflects at near glancing angles to horizontal surface 1450.

Fig. PHO-14 together with Fig. PHO-03 illustrate a previously unrecognized opportunity to take advantage of shadows naturally created by Lambertian reflecting surfaces with certain advantageous geometrical arrangements. Referring first to Fig. PHO-03, the fraction of Lambertian reflectance and its complement, fractions of specular reflectance are presented as a function of AOI for four example Lambertian surfaces 0330, 0340, 0350, and 0360. At angles of incidence on X-axis 0310 from 0° to about 45° common diffuse reflective surfaces are dominated by Lambertian reflectance plotted on Y-axis 0320. As AOI increases above about 45° a specular reflection component plotted on Y-axis 0325 increases at the complimentary expense of Lambertian reflection plotted on Y-axis 0320. Referring to Fig. PHO-14 it is apparent that Lambertian reflection is heavily weighted toward low angles of incidence and importantly a very small fraction of incident photons is diffusely reflected at high angles of reflection, that is, angles that are close to parallel to the reflective surface.

### Shadow—Independent Claim

A system and method to increase photonic persistence inside a target chamber by locating at least one LRS in a shadow.
Shadow value is less than 90%, 75%, 50%, 25%, 10%, 5%.
Shadow creates a photon rich zone (PRZ)
Shadow/PRZ+clock shadow
Shadow/PRZ++teeth
Shadow/PRZ+−teeth
Shadow/PRZ+partial cylindroids
Shadow/PRZ+deflection shadow
Shadow/PRZ+virtual shadow
Shadow/PRZ+attenuation shadow
Shadow/PRZ+Lambertian cosine shadow
Above+any combination of passive and active shadows
Above+pre-polisher
Above+pre-polisher (HEPA)
Above+post polisher
Above+purge/dilution

### Photon Vortex (PV)

With very high specular reflectivity, particularly at high angles of incidence, photons can be introduced tangentially to the inside of a generally cylindrical or prismatic photon vortex target chamber polisher and at least some photons will reflect until attenuated. Preferably at least one of two ends of a target chamber cylinder are curved inwardly, preferably in a hyperbola with one asymptote defined by an inside diameter of a cylindrical or polygonal profile and a second asymptote orthogonal and along a radius of a cylindrical target chamber polisher. A hyperbola shaped surface nudges photons back toward an oval or prism cross-section nearer the center of a target chamber. In other embodiments a hyperbola-shaped surface can be replaced with parabolic or similarly curved surfaces, with a conical profile, with a flat plane approximately perpendicular to the axis, or combinations thereof. In each case such shapes urge photons that strike an inward facing or slanting surface to reflect toward a portion of a volume distal from both ends of an elliptical or prismatic target chamber. Figs. PHO-06 and PHO-09 provide sectional views of examples of photon vortex target chambers.

A preferred fluid inlet and outlet arrangement is tangential ingress and/or tangential egress in a sawtooth cylinder. See section entitled Clock Shadow. Limited to specular reflection an even more preferred fluid inlet and outlet location is located within a virtually shadowed central portion of a cylindrical or prism target chamber volume. Figs. PHO-07, PHO-10A PHO-11A and PHO-11B provide sectional views of examples of such shapes. Table 1 provides selected values (columns) for selected prismatic photon vortices (rows) with sidelength equal to L_{S}=1. See Fig. PHO-10A for an illustration of some terms of a prismatic regular hexagon. The column labeled “Adjacent non-acute faces” provides the number of adjacent faces that can be directly illuminated in a photon source cone without encountering an acute angle. The column labeled “Vertex to diagonal” provides a length from a midpoint of a diagonal to a closest vertex. The column labeled “Central virtual shadow polygon radius” provides a radius of a polygon formed from intersections of all vertex diagonals. A photon-sparse polygon is a shadow if a cone width of a photon source is about equal to an acute vertex angle and the most parallel edge-ray is approximately parallel to a face adjacent to a photon source.

Diffuse reflectors as illustrated in Fig. PHO 03 become more and more specular as angle of incidence approaches 90°. Accordingly, a photon vortex may be created with specular reflective materials, diffuse reflective materials, or a combination of specular and diffuse reflective materials.

### Photon Vortex (High Angle of Incidence)—Independent Claim

A system and method to increase photonic persistence by urging photons on paths that maintain high angles of incidence on reflective surfaces until photons strike a target or are otherwise attenuated.
Targets are T1K; Targets are oxidizable T3K
Photon Vortex+reflective surface=specular reflector
Photon Vortex+reflective surface=diffuse reflector
Photon Vortex+specular/diffuse reflector+cylindroid/prism
Above+any combination of passive and active shadows
Above+pre-polisher
Above+pre-polisher (HEPA)
Above+post polisher
Above+purge/dilution

### Photon Rich Cavity (Specular)

Another non-limiting example of a geometry which creates a photon rich zone is a pair of generally spherical mirrors that are aligned with some precision so that two reflecting surfaces point substantially at each other. The mirror profiles may be generally circular, elliptical, polygonal or any shape that meets the reflectivity requirements outlined below. In a first embodiment at least one laser introduced photon in a path that is close to parallel with the axis between the two mirrors but biased inwardly toward the central axis of a mirror pair to clear an edge of a first mirror and strike adjacent a periphery of a second mirror. Such a beam will reflect repeatedly between two mirrors until the photons are scattered, transmitted, or absorbed. This back-and-forth reflection process is sometimes referred to as ringing. The photon rich cavity (PRC) a subset of a photon rich zone (PRZ) of this first example would be nominally a cylinder stretching between the two opposing mirrors with a diameter approximately equal to a beam aperture. When operating, a PRC cavity encloses a PRC beam of bidirectional photons. That is, at any moment, about half of photons are moving toward a first mirror and the remainder of photons are moving away from a first mirror. The cylinder ends of a PRC beam are negatives of the two mirrors. Alternatively, at least two mirrors may be slightly off axis so that an introduced laser beam is parallel to an axis of focus of the two mirrors. In this arrangement the laser beam passes parallel but outside of the outside diameter of a first mirror and then strikes a peripheral edge of a second mirror which is offset to just intercept the incident laser beam. The two mirrors of a mirror pair need not be identical. The second mirror may be slightly larger and slightly more concave such that an incident beam feeding an optical cavity is essentially parallel to an axis of a mirror pair. More than two mirrors may be used to shepherd photons over more complex trajectories to create a folded cavity.

Although this narrative borrows some of the language used for cavity ring down (CRD) measurements, the present invention is not limited by the designs utilized for those CRD measurements. CRD mirrors are specifically designed for photons to be introduced at or near the center of their mutual optical focus. In the present invention, photons are introduced into a PRC on a path:
a. parallel to a mirror pair axis and outside the periphery of at least one of a plurality of mirrors, and/or
b. at close-to-parallel angles, where the angles of reflection from a first mirror surface cause at least some of the reflected photons to strike a reflective mirror surface of a second mirror and where subsequent reflections substantially remain within a PRC, and/or
c. at parallel or close to parallel angles of a mirror pair optical axis, within a cone of mutual optical focus and from an orifice, where said orifice is in a shadow.

Fig. PHO-15 provides a third example of a section view of optical cavity 1500, also called a focal cone, illuminated from photon source 1550 located outside focal cone 1500 of a typical concave-convex optical cavity mirror arrangement. The central portion of a smaller right mirror 1502 is generally spherical convex with a focal radius of L-R1, where L is the separation of a mirror pair and R1 is a focal radius of curvature of a larger second concave mirror 1501. The peripheral portion of mirror 1502 is concave to urge photons into a focal cone of optical cavity 1500. In Fig. PHO-15 first mirror 1502 possesses a lesser overall diameter than second mirror 1501 thus allowing a first angle of incidence 1510 from photon source 1510 to reflection point 1591, to be closer to 0° than would be the case if mirror 1502 were of a larger overall diameter. Photon source 1510 emits at least one photon along ray 1503 generally toward a first reflection point 1591. At least one photon's angle of reflection equals its AOI and reflects toward 1592. A peripheral portion of mirror 1502 is concave urging incident photon(s) into the focal cone 1500 and on a ringing trajectory toward point 1593 and onto point 1594. At least one photon is thus trapped in optical cavity 1500 and rings until it is attenuated through mirror encounters or when it strikes a target.

Cavity ring down or CRD is typically applied to imaging optics, where concavity and convexity is tightly prescribed as spherical mirrors. A mirror suitable for the present invention need not focus an image and hence its curvature is designed specifically to be forgiving of photons that enter a CRD focal cavity peripherally and/or at small acute angles. Further, relative flatness (a departure from sphericity) near the center of the mirror expands the diameter of a cavity facilitating the intersection of an air flow to be treated within said cavity.

The diameter of a photon rich cavity can be scaled to the limits of manufacturing technology. In one preferred embodiment a mirror pair resembles a pair of ordinary kitchen plates. The central part of such a mirror is essentially flat, and mirror edges are concave. Photons reflect back and forth between the two flat surfaces and walk to a periphery. Because two mirrors cannot be perfectly aligned and an incident light source cannot be perfectly parallel to an axis, after repeated reflections every photon will move or walk toward an edge. When a photon reaches the periphery, a concave shape at the periphery nudges said photon to veer back toward an opposite mirror edge. Photons are trapped on a plate mirror pair until they strike a target or are absorbed when incident upon a mirror.

Photonic efficiency of a laser fed PRC may obviate a need for optical shielding for protectees, if protectees are prevented from physically interacting with a PRC beam.

Another embodiment of a PRC substitutes approximately parallel photon rays for a laser. A section view of a right member 1602 of a pair of PRC mirrors together with a photon source 1605 and two non-imaging elements is illustrated in Fig. PHO-16. A first non-imaging element is a nominally parabolic reflector 1601. A second non-imaging element is conical diverting mirror 1603 which is back-to-back with mirror 1602.

Two exemplary ray traces are illustrated emanating from photon source 1605. Ray 1610 directly strikes nominally parabolic reflector 1601 and is reflected on an intercept path 1611 to an unseen left mirror of an optical cavity. Ray 1615 strikes conical diverter mirror 1603 and is deflected as ray 1616 to non-imaging nominally parabolic reflector 1601 where it is reflected as ray 1617 toward an unseen left mirror. While a first fraction of photons from photon source 1605 may miss an unseen left mirror entirely, and a second fraction may strike a left mirror at angles that prevent them from ringing within a PRC, a third fraction, which is unity less the sum of the first and second fractions, will ring within a PRC. Generally parallel streams of photons can be generated with optical elements such as but not limited to parabolic reflectors. Optionally other optical elements can be deployed around at least one PRC mirror to redirect at least some of the first and/or second fractions in almost parallel paths into a PRC and thus decrease an effective sum of first and second fractions.

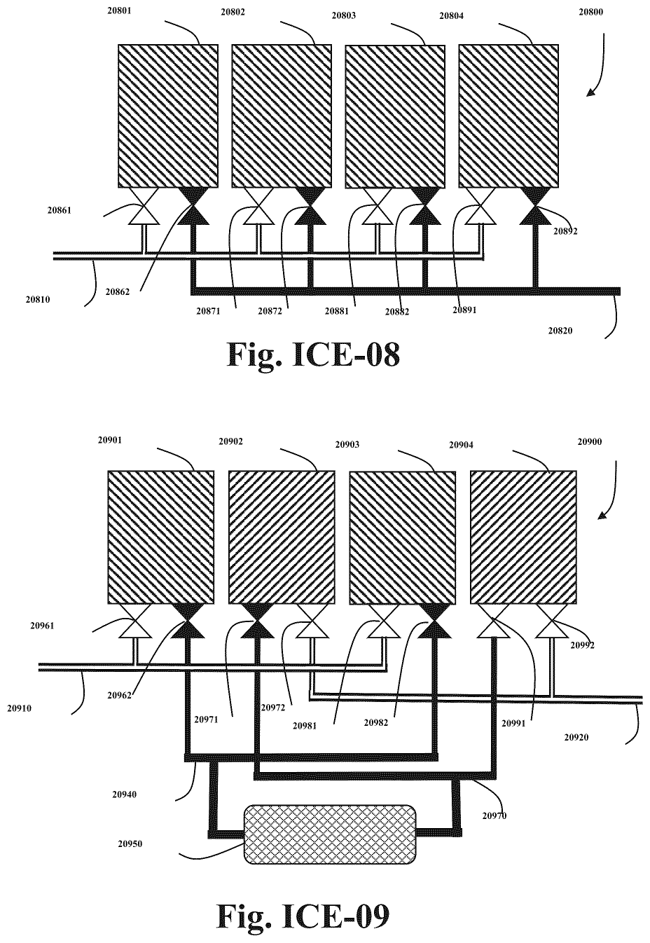

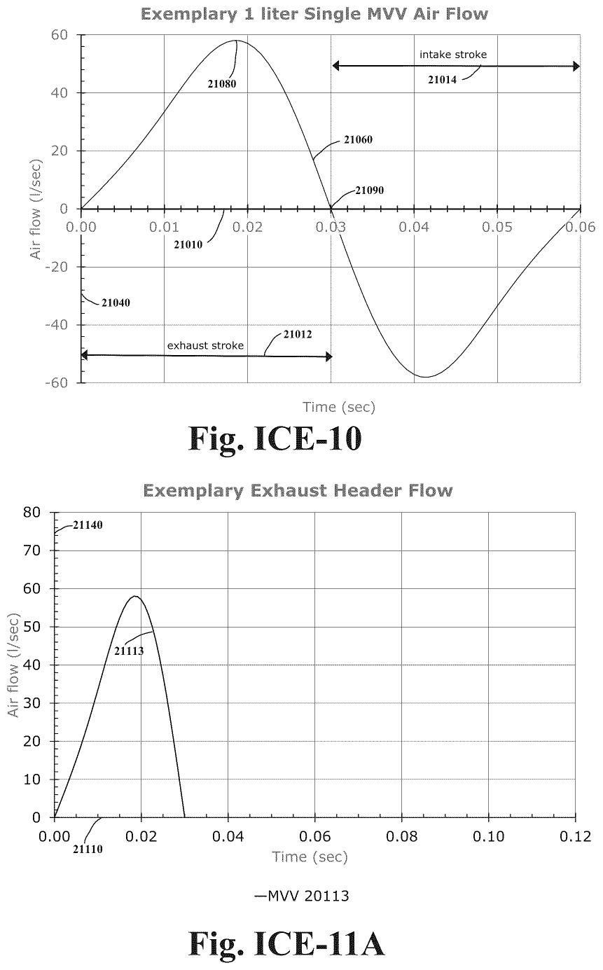

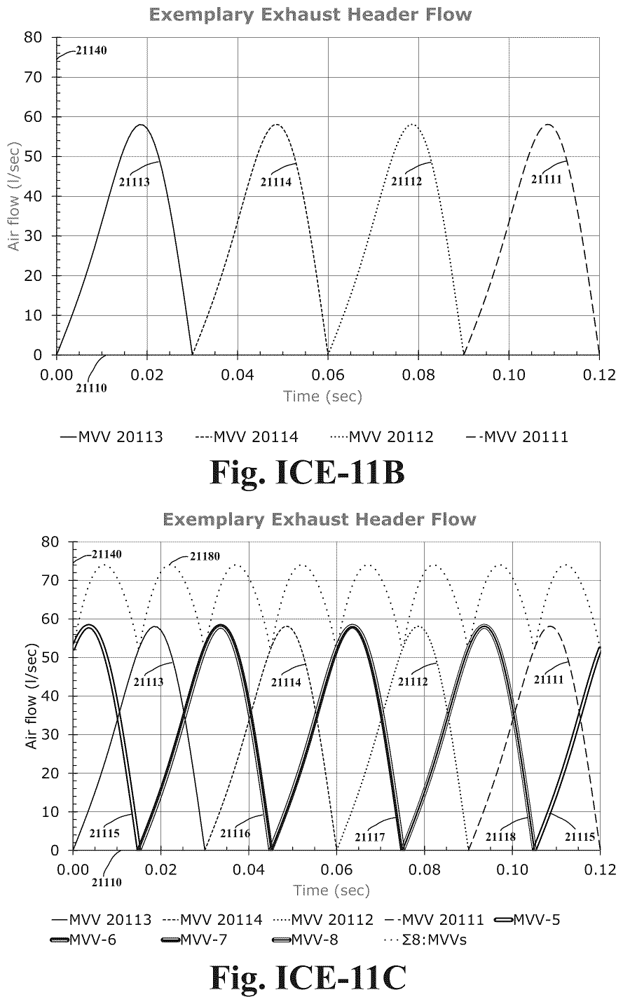

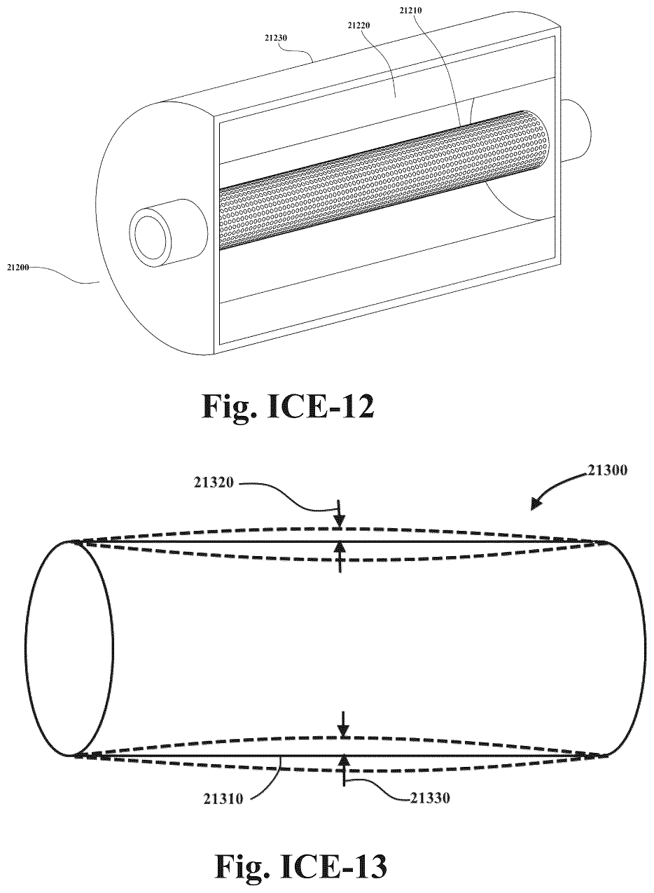

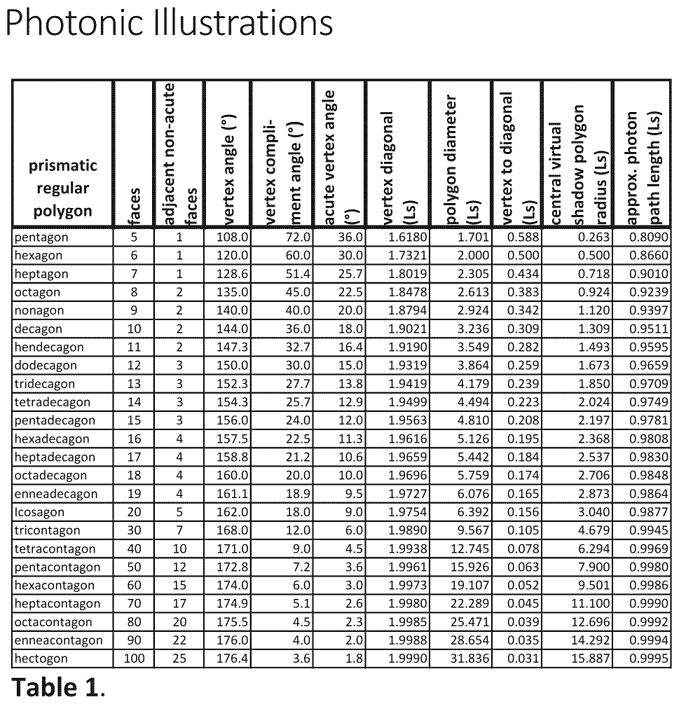

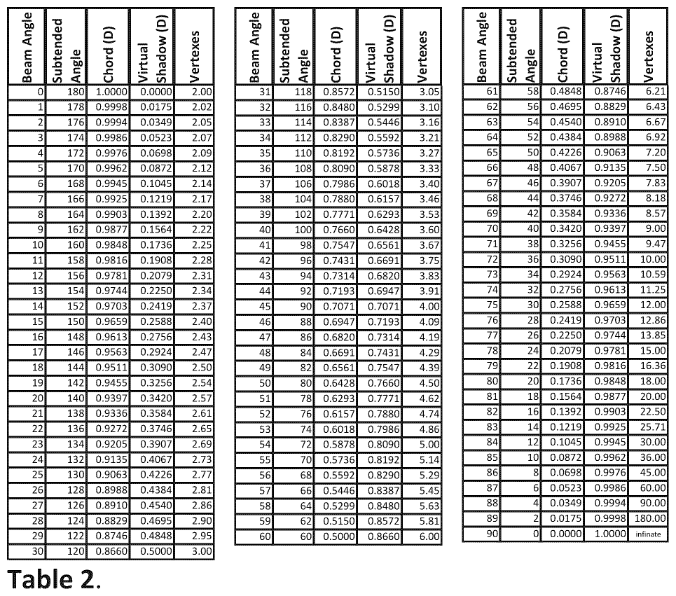

A third embodiment of a PRC introduces photons from at least one orifice in at least one mirror. Such orifices are disclosed by Prystupa and Pacak. See US 2022/0016306 A1 FIGS. 20A & 21B and their accompanying descriptions. This third embodiment improves upon the Prystupa and Pacak orifice by positioning an orifice within a passive optical shadow. In one embodiment of the present invention a deflection shadow is created by shaping at least one of at least two opposing mirrors to dissuade photons from returning to their source.

Figs. PHO-17A, PHO-17B, and PHO-17C present three sectional views of a non-limiting example of photon rich cavity 1700. Photon rich cavity 1700 is an annular volume between a frustum of an outer cone delineated on its outside by a surface of revolution of ray 1713 or ray 1715 and a frustum of an inner cone delineated on its outside by a surface of revolution of ray 1712 or ray 1714. Orifice 1790 near the center of mirror 1720 falls within passive optical shadow 1730. In Fig. PHO-17C passive optical shadow 1730 is a deflection shadow. Partially collimated photons from photon source 1701 are introduced into PRC 1700 through orifice 1790 in Fig. PHO-17A. The partially collimated beam is represented by two edge rays, 1703 and 1705, and two exemplary interior rays, 1702 and 1704. Interior rays 1702 and 1704 are adjacent to mirror pair axis 1750 in PHO-17A. Ray 1703, ray 1705, ray 1702 and ray 1704, reflect from the upper, convex mirror 1710 in Fig. PHO-17B and are reflected downward to a predominantly concave non-imaging mirror as ray 1713, ray 1715, ray 1712, and ray 1714 respectively. Predominantly concave mirror 1720 has a convex portion 1706, best illustrated in Detail B of Fig. PHO-17B which is concentric with mirror pair axis 1750 and PRC 1700. Convex mirror portion 1706 creates a deflection shadow proximate orifice 1790. Convex portion 1706 deflects incident ray 1712, incident ray 1714 and other incident rays proximate to but not between ray 1712 and ray 1714 slightly away from axis 1750 of mirror pair 1710 and 1720 and coaxial orifice 1790. Orifice 1790 is shadowed for all but photons incident from a first reflection of rays that lie between ray 1702 and ray 1704. The photon rich cavity 1700 is generally depicted as the shaded region in PHO-17C.

The only photons to strike orifice 1790 are those which lie between illustrated interior rays 1712 and 1714. For example, if an orifice diameter is 1 mm, an outer diameter of the photon rich zone 1700 is 100 mm proximate to mirror 1720, and a photon flux is distributed about equally throughout its radiation cone, 0.01% of the original photon flux will enter orifice 1790 and strike photon source 1701. Some photons that strike 1701 will be reflected and some absorbed. The single pass loss of less than 0.01% is inconsequential and even that loss is avoided on subsequent ringing reflections.

The PRC beams of the above described-mirror arrangements create essentially long cones or cylinders of photon rich volumes between mirror pairs. Substantially all designated air is carried by ductwork and constrained by bulkheads to flow through a PRC beam with methods well known in the art to create a target chamber polisher. Flow can be generally parallel to a PRC axis, perpendicular to a PRC axis, directed diagonally through a PRC axis, or directed to flow back and forth across at least one optical axis. For flow that has at least a component vector perpendicular to an optical axis, air baffles may be advantageously employed to equalize a cumulative fluence of photons on each portion of air that flows through a PRC. Non-limiting examples of fluence equalized flow may include concentrating baffles and vortex induction.

### Concentrating Baffles

Fig. PHO-18A illustrates a sectional view of an exemplary concentrating baffle design for perpendicular flow of air from a duct 1800 across conical PRC 1803. Photon flux in a PRC is approximately perpendicular to the sectional view of Fig. PHO-18A. A dose required to achieve a desired germicidal efficiency determines a desired fluid flow rate. The fluid flow may be continuous or intermittent. A cross section of duct 1800 may include but not be limited to rectilinear, trapezoidal, circular, polygonal, and ellipsoidal.

The area outside the circumference of PRC 1803 is in a passive shadow. This passive shadow is well suited for the introduction of designated air 1801 to be polished from flow path 10134 illustrated in Fig. APS-01A and a supply of polished air 1802 into flow path 10135 illustrated in Fig. APS-01A.

Baffles 1805, 1806, 1807, and 1808 direct incoming air 1801 such that a cumulative dose of each air velocity vector 1820, 1821, 1822, 1823, 1824, 1825, 1826, 1827, 1828, 1829, and 1830 within the PRC is approximately the same. This is accomplished by adjusting baffles such that each air velocity vector 1820, 1821, 1822, 1823, 1824, 1825, 1826, 1827, 1828, 1829, and 1830 divided by each individual chord length may be approximately equal, may be within a factor of 1.2, within a factor of 1.5, or within a factor of 2. Substantial departures including factors greater than two from equal quotients yield inconsistent single pass target treatment but does not depart from the spirit of the present invention as targets entrained in air within a PES may make multiple trips through a target chamber.

### Vortex Induction

A second method to achieve flux equalized flow is vortex induction and is illustrated in a mixed sectional-isometric view in Fig. PHO-18B. Vortex induction equalizes fluence by imparting angular momentum on designated air 1801 around flow axis 1850 employing a plurality of stationery turbine vanes 1860 through 1869. Each stationery turbine vane is fixedly attached to optional housing 1870 which nests fixedly and sealingly within circular duct 1890. Optional housing 1870 may be omitted and stationary turbine vanes may be fixedly attached directly to circular duct 1890. To assure somewhat uniform dosing the rotational speed of a vortex is preferred to be sufficient to complete at least one revolution while air flow within circular duct 1890 traverses along axis 1850 and collectively perpendicular to PRC axis 1804. While collective flow is perpendicular to PRC axis 1804, individual quanta of air each follow a generally helictical path. For example, if a PRC diameter is 0.2 length units and an air velocity is 1 length unit per second, an angular velocity of designated air is preferably at least 10π radians per second. To optimize the utilization of available photonic flux, inner diameter 1845 of fluid flow duct 1890 and inner diameter 1840 of PRC 1803 at their mutual crossing are preferably about the same. For example, a pair of plate-type mirrors having circular or polygonal diameters of about 0.5 length units could cross at about a right angle with a single circular or polygonal fluid duct of about the same diameter. While a vortex flow is best accommodated by a circular cross section, other cross section shapes remain within the scope of the present invention.

A mirror pair as illustrated in Fig. PHO-17C may have a mirror separation of 1 length unit and a conical diameter of 0.23 length unit near concave mirror 1720 and 0.2 length units near convex mirror 1710. In such a case a trapezoidal or rectilinear duct may deliver air to a substantial portion of PRC 1700. A second approach to encourage intersections of conical photon flux with air is to split designated air flow into a plurality of ducts and virtual ducts. A virtual duct is an approximate bulk flow path through a PRC unconstrained by walls. A virtual duct begins where an actual duct carrying designated air ends near a first PRC boundary and ends where an actual duct carrying polished air begins near a second PRC boundary. Fig. PHO-18C provides a non-limiting example where four designated air portions are allocated into four virtual ducts. Fig. PHO-18C retains all callouts from Fig. PHO-17C without number changes. A cross section parallel to flow axis 1850 of first duct 1890 and perpendicular to PRC axis 1804 is illustrated individually in Figs. PHO-18A and PHO-18B and has a diameter of about 0.23 length units. The same cross section 1890 is virtual duct 1890 and is illustrated in Fig. PHO-18C. The cross section of second virtual duct 1891 has a diameter of about 0.22 length units. The cross section of third virtual duct 1892 has a diameter of about 0.21 length units. The cross section of fourth virtual duct 1893 has a diameter of about 0.20 length units. Each of the four ducts preferably:
1. share approximately parallel axes,
2. are adjacent,
3. are stacked such that each duct terminates just short of PRC outer edges 1713 and 1715 at a PRC axial location that shares a similar diameter to that of the duct, and
4. has a flow rate of air that is proportional to its diameter to yield approximately equal dosing. This proportionality to diameter is a result of the air flow rate being proportional to the diameter squared and photon flux being inversely proportional to the diameter. The quotient of the diameter squared, and the diameter is equal to the diameter.

While some quanta of air flowing across PRC 1700 will move between a pair of virtual ducts between entering PRC 1700 and exiting PRC 1700, most air quanta will remain largely within the virtual duct from which it enters PRC 1700. Such exchanges between virtual ducts do not degrade the performance of a target chamber as in most cases these quanta remain in the PRC longer than an average quantum of air taking a shorter path.

### Photon Rich Cavity—Independent Claim

A system and method to create a photon rich cavity with at least one photon source and at least two mirrors where the focus of each mirror is directed at another and where air and air entrained targets are made to flow through photon rich cavity.
PRC where photon source is a laser.
PRC where photon source is an LED & focusing optics to create partially collimated rays.
PRC where photon source is an LED & focusing optics to create rays that diverge less than 10°.
PRC with photon source outside of at least one mirror diameter.
PRC with photon source inside of at least one mirror diameter via an orifice located in a deflection shadow.
PRC+>2 mirrors.
PRC+>1 mirrors+perpendicular air flow.
PRC+>1 mirrors+parallel air flow.
PRC+>1 mirrors+diagonal air flow.
Above+fluence equalized flow.
Above+fluence equalized flow-concentrating baffles.
Above+fluence equalized flow-vortex induction.
Above+pre-polisher
Above+pre-polisher (HEPA)
Above+post polisher
Above+purge/dilution

### Photonotron

### Annular Cylindroid Photonotron

Fig. PHO-19A provides a sectional view of an exemplary first type of photonotron 1900 with at least one photon source 1930. Photons produced by 1930 are collimated or partially collimated as ray 1901 which enter annular target chamber 1940 of photonotron 1900 via orifice 1950. Orifice 1950 is a negative sawtooth cylinder. Photons in this non-limiting example reflect between the inner cylindroid 1910 and outer cylindroid 1920 predominately in a counterclockwise direction. Inner cylindroid 1910 is suspended within outer cylindroid 1920 by axial member 1969 as shown best in Fig. PHO-19B.

Referring now to the ray details of Fig. PHO-19A, photon source 1930 preferably includes a non-imaging photon concentrator as described in Minimized Photon Source or a laser. Photon source 1930 delivers photons approximately tangential to the annular volume of a photonotron, and preferably in a shadow of a feature as described in Passive Shadowing. As taught in Angle of Incidence (Diffuse Reflection) even with diffuse reflectors a substantial portion of incident ray 1901 undergoes specular reflection and especially so with glancing and near glancing rays as documented with data compiled in Fig. PHO-03. As illustrated in Fig. PHO-19A, photon source ray 1901 experiences primarily specular reflection and this specular component is represented as ray 1902. Ray 1902 is properly represented in the two-dimensional view afforded by Fig. PHO-19A. Lessor portions of incident photons are scattered with a muted Lambertian cosine law in three dimensions. Almost all diffusely reflected photons reflect in a half-cone-shaped volume between 1902 (the center axis of a cone) and diffuse ray 1903 (a single line representing an edge of a cone in the plane of Fig. PHO-19A). The main ray, 1902, goes on to reflect again in a similar way. To avoid cluttering the illustration, subsequent reflections of 1902 are omitted. Ray 1903 strikes outer wall 1915 of inner cylindroid 1910 at a shallow glancing angle and again a majority of 1903 is propagated as specular ray 1905. A smaller portion, represented by ray 1904, is diffusely reflected slightly more outwardly than ray 1905. Again, to avoid cluttering the illustration we suspend illustrating future reflections of ray 1904, but it continues analogously to ray 1902. Finally, ray 1905, which is the largest portion of ray 1903 grazes inner wall 1925 of outer cylinder 1920 and a majority of 1905 undergoes specular reflection as ray 1906 and a minority of 1905 is conically distributed between 1906 and 1907. Hundreds or thousands of reflections like those described occur. Most of those reflections are specular and continue counterclockwise within annular volume 1940. Diffuse reflections maintain a generally counterclockwise flight path. The narrow annular clearance all but precludes photons from reversing clock direction and propagating clockwise in the plane of Fig. PHO-19A.

An annular cylindroid photonotron may be two nominal cylindroids and generally coaxial solids composed entirely of reflective materials or composed of a plurality of materials with laminations or coatings of reflective materials to improve reflectivity on surfaces proximate annular volume 1940. An annular volume of cylindroid ends may be enclosed with reflective rings, where surfaces are proximate to annular volume 1940. More preferred than reflective rings are an inward curvature of nominally cylindroid reflectors as illustrated in Fig. PHO-19B. Said inward curvature urges photons approaching cylindroid ends back toward a central portion of a photonotron as described in Photon Vortex, by other methods known in the art, or two cylindroids may merge into an ellipsoid as will be described in Toroidal Photonotron. Such curvatures on ends of two generally coaxial cylindroids are coordinated to maintain a clearance between opposing surfaces close enough to each other to prevent or inhibit photon clock reversal. Photon clock reversal occurs when a photon that is emitted in a first clock direction reverses to a second and opposite clock direction after at least one reflection. For example, a counterclockwise photon becomes a clockwise photon or vice-versa.

However, the plane depicted by Fig. PHO-19A does not illustrate the third dimension—the z-dimension perpendicular to the plane of Fig. PHO-19A. Consider for example a photon that undergoes repeated diffuse reflections always making a maximum +z-turn at each reflection and imagine that a maximum z-turn was 18°. This +z-biased photon would reverse its direction 180° after 10 such consecutive reflections if a cylindroid axis was very long and hence a photon failed to encounter a cylindroid wall or an inward surface curvature that urged a photon back towards its original counterclockwise direction. A photon that undergoes a clock reversal may partially illuminate a shadow. A feature of photons cycling around in a prescribed clock direction (counterclockwise in Fig. PHO-19A, but clockwise is equally as effective) allows passive clock shadows to be created.

In Fig. PHO-19B and proximate ends of photonotron 1900, at least one fluid inlet orifice 1971 delivers designated air from flow path 10134 illustrated in Fig. APS-01A and at least one fluid outlet orifice 1972 provides polished air into flow path 10135 illustrated in Fig. APS-01A. Inner cylindroid 1910 is suspended in an interior of outer cylindroid 1920 with axial member 1969. Axial member 1969 and outer cylindroid 1920 are tied to at least one common bulkhead 1917 and preferably to a second bulkhead 1918. Bulkhead 1917 is in airtight fluid communication with flow path 10134 of Fig. APS-01A and delivers designated air to target chamber 1930. Bulkhead 1917 includes at least one fluid pathway such as perforation 1973 to facilitate fluid communication through orifice 1971. Bulkhead 1918 is in airtight fluid communication with flow path 10134 of Fig. APS-01A and delivers polished air from target chamber 1930. Bulkhead 1918 includes at least one fluid pathway such as perforation 1974 to facilitate fluid communication through orifice 1972.

Photon source 1930 provides a cone-like dispersion of photons represented by edge rays 1975 and 1990 in the plane of Fig. PHO-19B. Because edge rays 1975 and 1990 are introduced at high angles of incidence in the plane perpendicular to Fig. PHO-19B to diffuse reflective surfaces 1915 and 1925 a substantial portion of reflected photons undergo specular reflections. Edge ray 1990 is predominantly reflects as ray 1991 toward orifice 1971. Ray 1991 predominately reflects as ray 1992 toward orifice 1971. Ray 1992 predominately reflects as ray 1993 away from orifice 1971. Ray 1993 undergoes subsequent reflections and becomes ray 1994, 1995, 1996, 1997, 1998 and 1999. In each case, heading away from orifice 1971 and toward orifice 1972. Edge ray 1975 is tracked in a similar way from photon source 1930, however edge ray 1975 has an initial trajectory toward orifice 1972. Reflection angles between rays 1976 through ray 1979 become larger. After ray 1975 passes axis center 1970 each subsequent reflection angle between ray 1980 through 1986 becomes smaller while still moving toward orifice 1972 until ray 1987 reverses direction and reflects away from orifice 1972. The curvatures of annular interior surface 1915 and annular interior surface 1925 are judiciously chosen to cause each reflection pair between annular interior surface 1915 and annular interior surface 1925 to nudge a photonic trajectory away from orifice 1971 and away from orifice 1972 and toward axis center 1970 as photons approach orifice 1971 and orifice 1972 respectively. These reflections continue until photons are absorbed during a reflection or they strike a target.

At least one photon source 1930 is preferably located outside of photonotron 1900 and includes optics to introduce photons in a collimated or partially collimated beam. A photon beam is introduced into target chamber 1940 through a minimally sized orifice 1950. Orifice 1950 is located in a clock shadow best illustrated in Fig. PHO-19A and a virtual shadow best illustrated in Fig. PHO-19B as photons are urged to concentrate near axis center 1970.

A photonotron may be used instead of Photon Vortex utilizing predominantly diffuse reflective surfaces. For predominantly specular reflection there may not be a need for the inner cylindroid. Highly specular reflection may require elaborate manufacturing processes, such as reflective coating deposition on substrates such as glass or plastic, utilizing process including but not limited to ion-beam deposition (IBD), ion-assisted deposition (IAD), and ion-plating. Diffuse reflective surfaces or lower tolerance specular reflective surfaces in large, curved shapes enjoy certain economic advantages.

### Cylindroid Photonotron

A cross section of an exemplary second type of photonotron, a cylindroid photonotron 2000, is illustrated in Fig. PHO-20A. Photon source 2002 located outside cylindroid photonotron 2000 communicates via negative sawtooth orifice 2005 to interior target chamber 2001. A collimated or partially collimated beam 2010 is incident upon an inner reflective wall 2020 of cylindroid photonotron 2000. When beam 2010 interacts with inner reflective wall 2020 at point 2021 a first larger fraction of incident photons undergoes specular reflection to an extent illustrated by Fig. PHO-03, a second smaller fraction may be transmitted through a reflective material, a third fraction may be absorbed or attenuated by a reflective material, and a fourth, remaining fraction, will be reflected generally following Lambert's cosine law illustrated in Fig. PHO-20A and represented as a diffuse distribution or pie slice d1. The first portion is represented in Fig. PHO-20A as specular ray s1. Ray s1 continues near the inside surface 2020 of photonotron 2000 until it strikes inside surface 2020 at point 2022. Ray s1 splits into its four portions and two of those portions are illustrated in Fig. PHO-20A analogously to interactions described infra at reflection point 2021.

Returning to the diffuse reflectance at point 2021 and the fourth portion of a reflection of ray 2010, is represented as pie slice d1. A single exemplary vector of diffuse distribution d1 is illustrated as d1*. The diffusely reflected light ray d1* interacts with inner reflective wall 2020 at point 2023. Ray d1* interacts with inner reflective wall 2020 at reflection point 2023 and is divided into four portions analogous to a distribution at reflection point 2021, namely a specular portion s3, a non-illustrated transmitted portion, a non-illustrated absorbed portion, and a diffuse portion d3 with an exemplary ray d3*. Each of these rays and all rays of Fig. PHO-20A propagate indefinitely until attenuated to zero by interactions with inner wall 2020, target strikes, or leakage from an orifice.

The orifice 2005 enjoys three shadow effects. Due to specular reflection of a high AOI beam on inside surface 2020 photons circulate predominantly in a counterclockwise direction in cylindroid photonotron 2000 yielding a clock shadow at orifice 2005. The counterclockwise bias of photons results at least in part from an attenuation shadow. Diffuse reflections tend toward angles normal to an inner circumference of reflective wall 2020, but such reflections generally occur after at least some specular reflections and thus are attenuated. A third shadow effect is a Lambertian cosine shadow. Reflective flux tangential to the inner surface is much less intense than reflective flux at lower angles of incidence.

In Fig. PHO-20B and proximate ends of photonotron 2000, at least one fluid inlet orifice 2071 provides fluid communication with designated air from flow path 10134 illustrated in Fig. APS-01A and at least one fluid outlet orifice 2072 provides polished air into flow path 10135 illustrated in Fig. APS-01A. Photonotron 2000 is a essentially a solid of revolution around axis 2069.

Photon source 2002 provides a cone like dispersion of photons represented by edge rays 2050 and 2060 in an axial dimension. Because edge rays 2050 and 2060 are introduced at high angles of incidence to diffuse reflective surface 2020 in the plane of Fig. PHO-20A, a substantial portion of reflected photons undergo specular reflections. Edge ray 2050 predominantly reflects as edge ray 2051 and is biased toward orifice 2072. Ray 2051 predominately reflects as ray 2052 and has reversed its axial bias and is now heading toward orifice 2071. Ray 2052 predominately reflects as ray 2053 and remains heading toward orifice 2071. Ray 2053 predominately reflects as ray 2054 and remains heading toward orifice 2071. Ray 2054 predominately reflects as ray 2055 and has reversed its axial bias again and is now heading toward orifice 2072. Except for a ray perpendicular to axis 2069 at axis midpoint 2070, the curvature of interior surface 2020 causes each reflection to nudge a photonic trajectory away from orifice 2071 and away from orifice 2072 and toward axis midpoint 2070. These reflections continue until photons are absorbed during a reflection, leak from an orifice, transmit through a reflective surface, or strike a target. Edge ray 2060 takes a slightly different path than edge ray 2050 but follows a similar pattern and is nudged away from orifice 2071 and orifice 2072 and toward axial mid-point 2070.

### Toroidal Photonotron

Fig. PHO-21A is a simplified section view of toroidal photonotron 2100. In one embodiment of the present invention photons are constrained in two dimensions within toroidal photonotron 2100 to all but eliminate photonic clock reversals ensuring photons cycle predominately in prescribed clock direction 2104. Sectional detail (Section A-A) of toroidal photonotron 2100 illustrates its elliptical cross section 2140. Reflecting surfaces 2150 of toroidal photonotron 2100 may be predominately diffuse, predominately specular, or partially diffuse and partially specular. Outer housing 2155, inner housing 2156, bulkhead 2157 together with housing annular end caps 2160 and 2161 and bulkhead 2157 illustrated in Fig. PHO-21B enclose toroidal target chamber 2130 to shepherd designated air from flow path 10134 illustrated in Fig. APS-01A into treatment chamber 2130 via at least one inlet 2115 and at least one manifold 2110 and polished air from at least one manifold 2120 and at least one orifice 2125 into flow path 10135 illustrated in Fig. APS-01A.

Driven by a pressure differential, air is urged to follow flow path 2101 and is deflected by baffle assembly 2105 to flow counterclockwise through toroidal interior 2130 until it again encounters baffle assembly 2105 and exits into manifold 2120 as illustrated by flow path 2102. Manifold 2110 and manifold 2120 are not in direct fluid communication with each other except as just described. Outer housing 2155 and inner housing 2156 together with housing annular end caps 2160 and 2161 create a non-concentric annular cylindrical volume. Said non-concentric annular volume is divided into two portions, namely manifold 2110 and manifold 2120 by bulkhead 2157, baffle assembly 2105, and toroidal target chamber 2130. Elliptical cross section 2140 of photonotron 2100 makes an airtight seal with housing annular end caps 2160 and 2161. Exterior elliptical cross section 2140 of photonotron 2100 makes an airtight seal with bulkhead 2157. Baffle assembly 2105 is illustrated in detail in Fig. PHO-21C.

Baffle assembly 2015 includes three components. Optically clear window 2108 is sized to match the inside dimensions of toroidal interior 2130 and allows photons to pass through with minimal absorption. Window 2018 is supported by airtight connection 2174 with first baffle support 2106 and by its airtight connection 2172 with toroid interior surface 2150. Baffle support 2106 is in airtight connection with inside surface of housing 2155 at surface 2176. Baffle support 2106 is in airtight connection with housing annular end caps 2160 and 2161 at edges 2175 and 2179. Second baffle support 2107 completes fluid-tight connections with interior housing 2156 at edge 2170, housing annular end caps 2160 and 2161 at edges 2177 and 2178, first baffle support 2107 and edge 2173, and with toroid outward-facing exterior 2151.

Manifold 2110 and manifold 2120 are in indirect fluid communication through a common volume of PES 10115 in Fig. APS-01A. Manifold 2110 receives designated air from at least one orifice 2115 from flow path 10134 illustrated in Fig. APS-01A. From manifold 2120 air is withdrawn from at least one orifice 2125 providing polished air into flow path 10135 illustrated in Fig. APS-01A.

Photons are tangentially introduced at photon source 2103 and travel indefinitely through toroidal volume 2130 until photons are absorbed by a target, during transmission through window portion 2108 of baffle 2105 or reflecting surface 2150. Section A-A of Fig. PHO-21A illustrates a section of the toroid—an ellipse with a major axis and a minor axis. A major axis is greater than or equal to a minor axis. The ratio of a major axis to a minor axis is greater than or equal to unity and is a toroidal profile. A toroidal profile and its nominal cross-sectional area are chosen to satisfy two criteria.
1. Photon clock reversal is less than 25%, 10%, 1%, 0.1%, or 0.01%.
2. Desired air flow rates are achieved without excessive fluidic power loss or unacceptable noise.

Window portion 2108 of baffle 2105 is optically transparent to photon wavelengths created by at least one photon source 2103. Exemplary transparent materials for UV photons include polymers like acrylic and silicone, ceramics and glasses like quartz and fused silica, artificial diamond, and specialized UV glass compositions well known in the art. Optical transparency greater than about 90% is desirable. The baffle angle relative to an average photonic direction 2104 is chosen such that incident photons from photon source 2103 and from photonotron toroid volume 2130 are not reflected. An optically transparent baffle is airtight. While Fig. PHO-21A illustrates a single sawtooth where photons are introduced, air is supplied, and air is withdrawn, other configurations with more than a single sawtooth are included in the scope of the present invention.

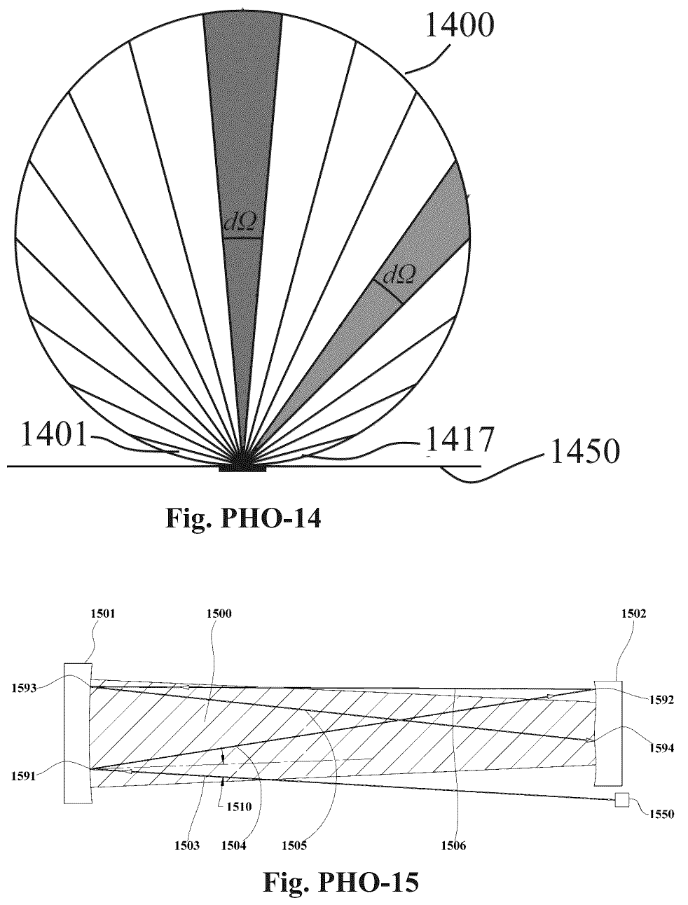

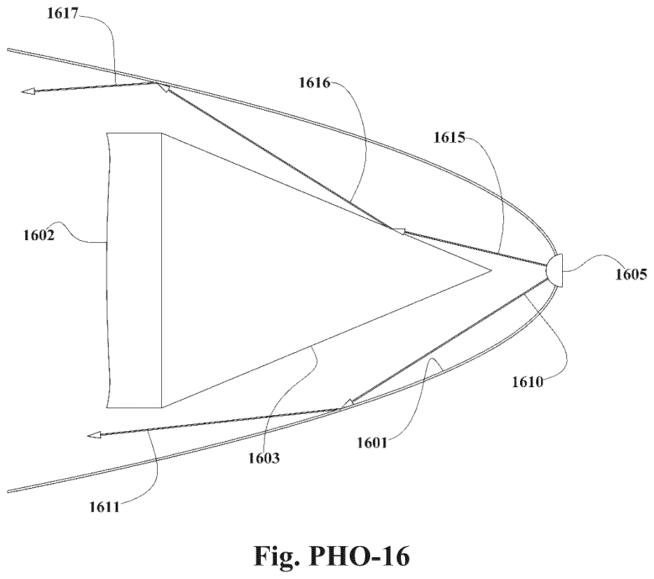

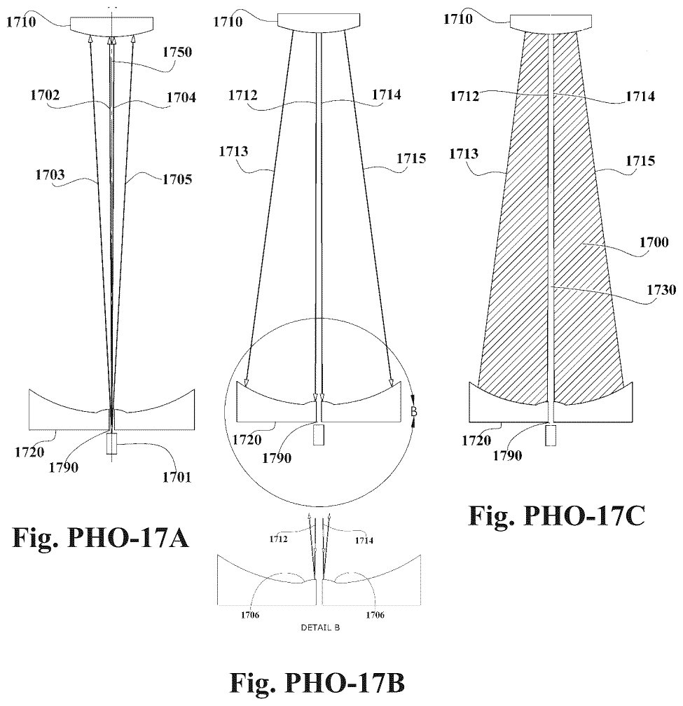

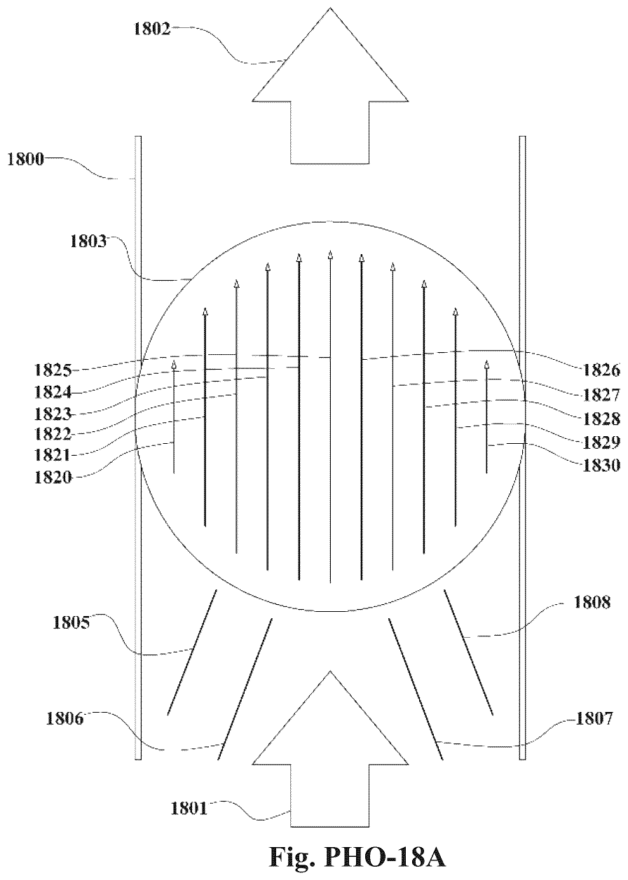

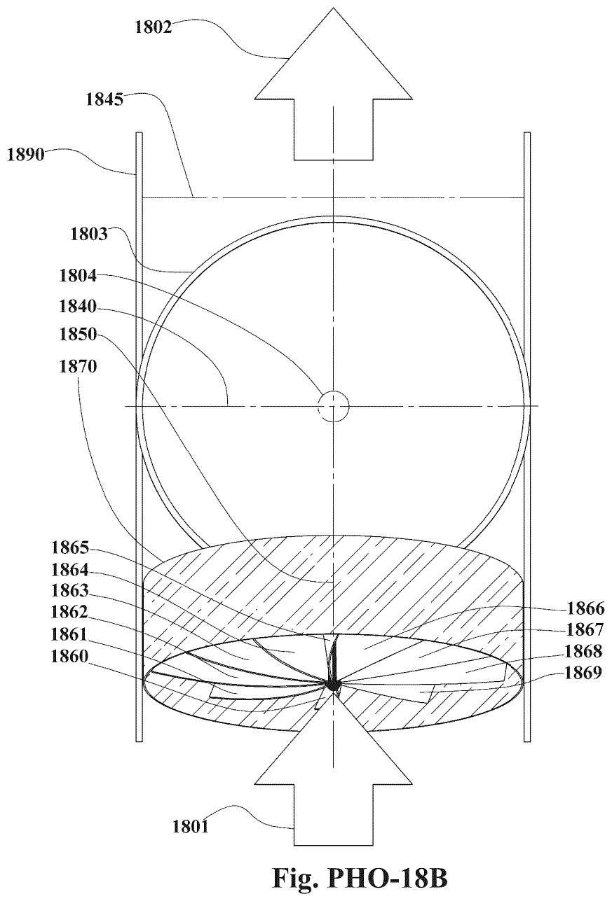

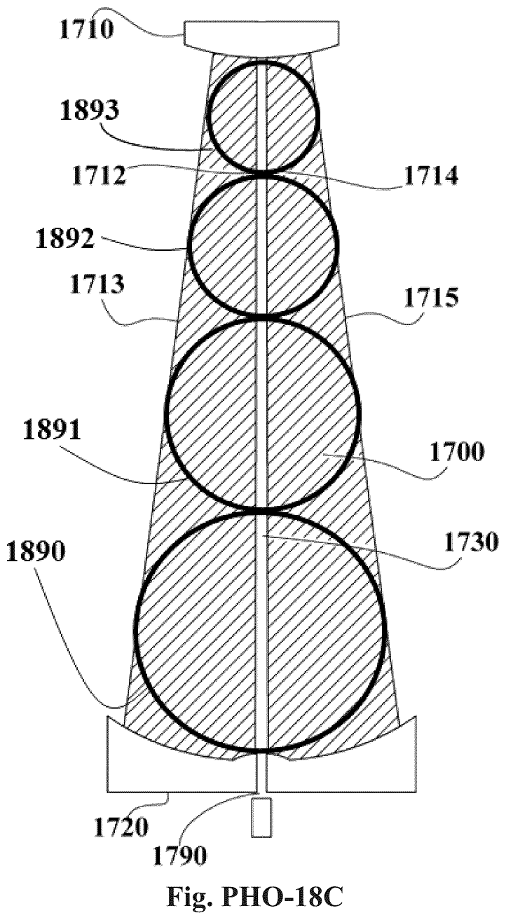

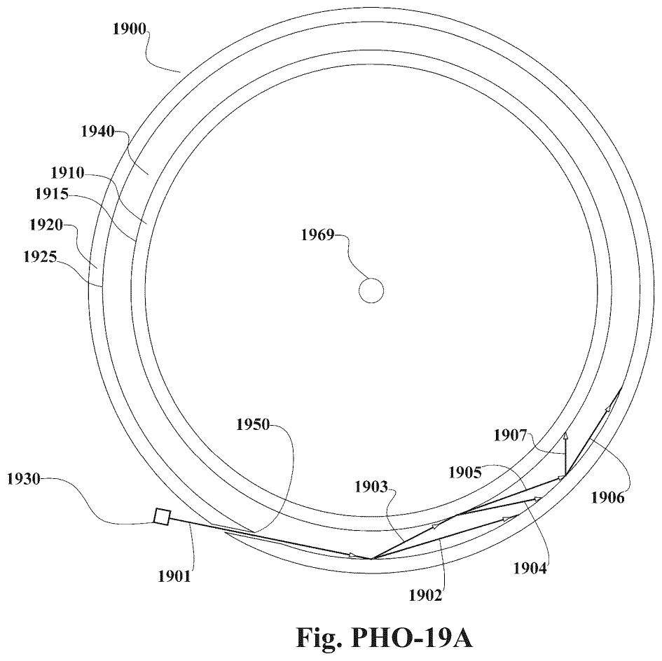

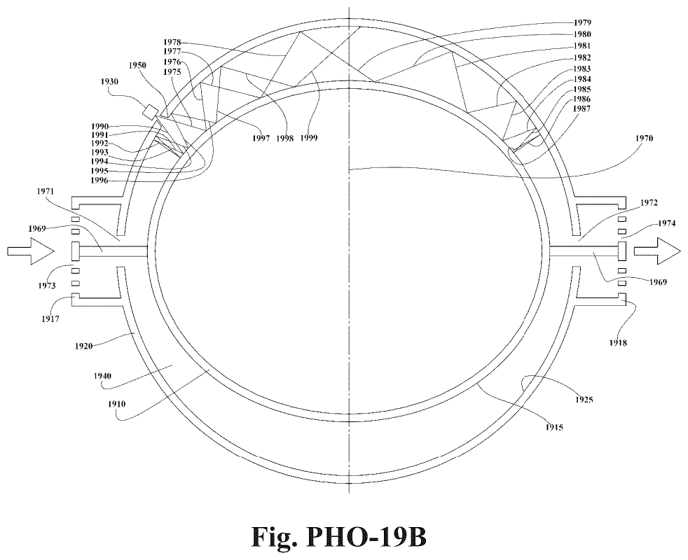

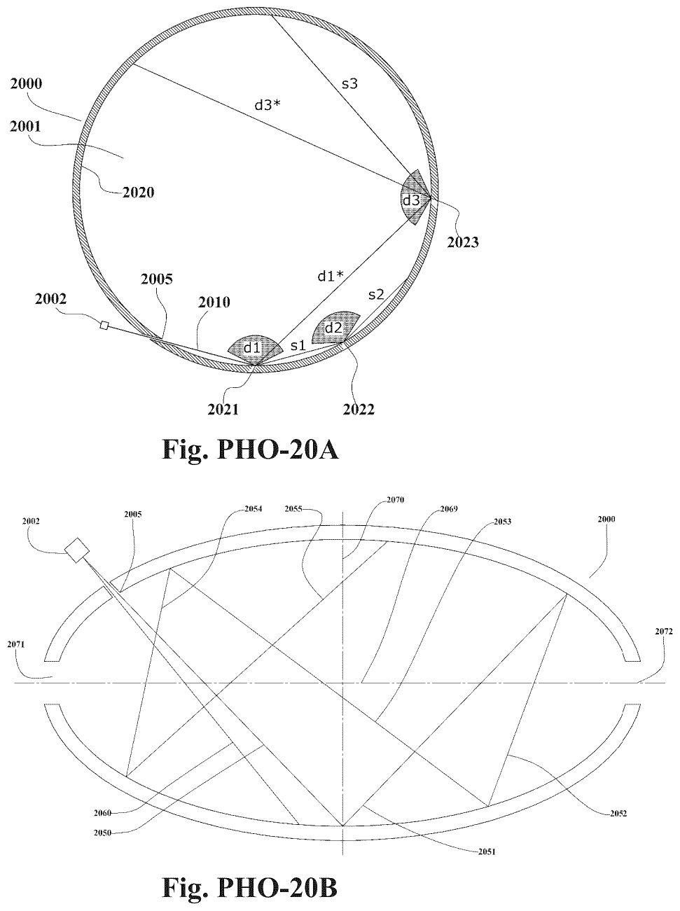

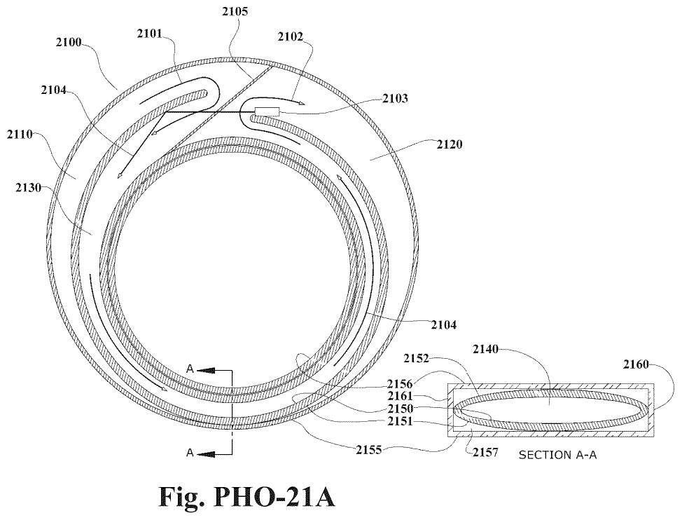

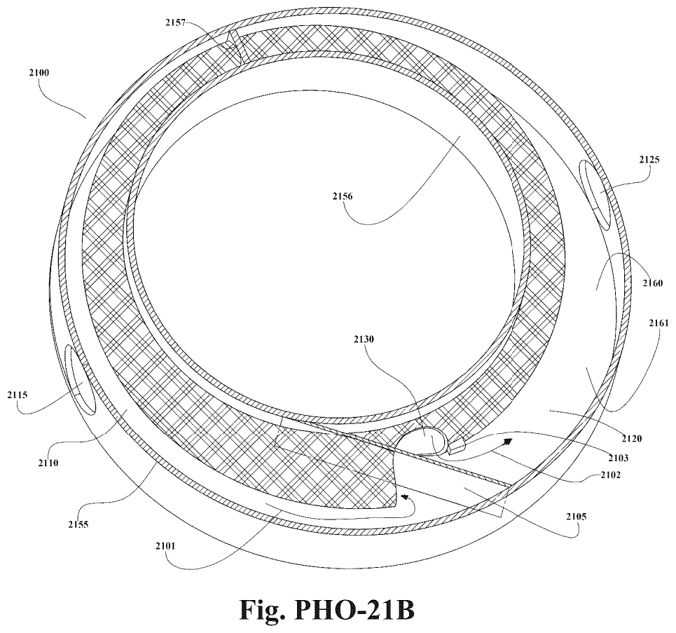

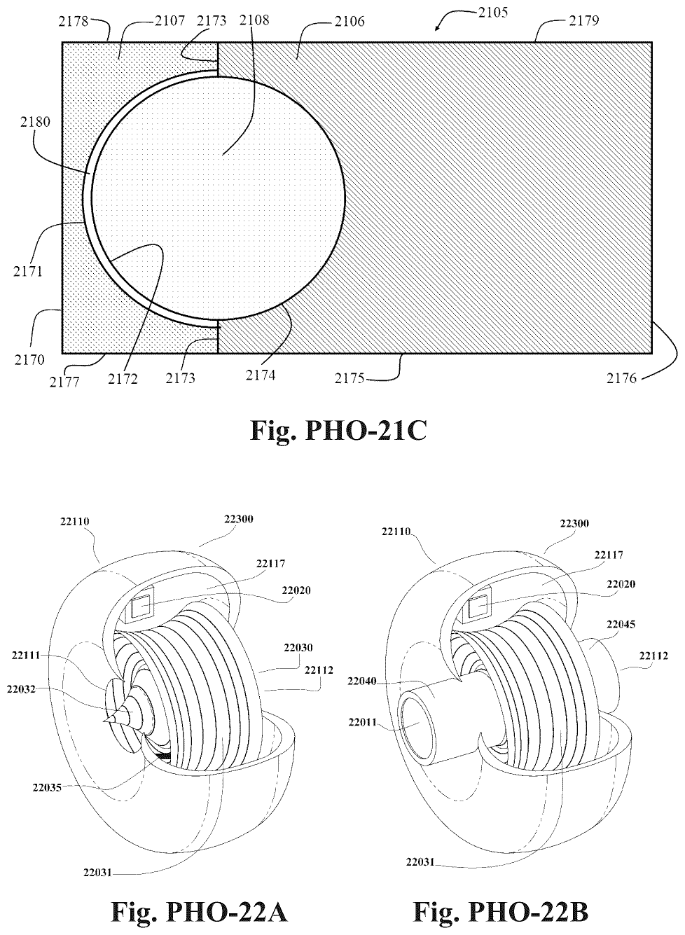

A second example of a toroidal photonotron 22300 with an alternate means to introduce and withdraw air is illustrated by Efrati (GB2593827A, Fig. 4 and Fig. 5) in Figs. PHO-22A and PHO-22B where each numeric call out has been provided a prefix of “22” for three-digit callouts and “220” for two-digit callouts. There are three differences between Efrati's illustration and the present invention.
1. Efrati's “compact light-shielded trap” 22300 utilizes specular reflective surfaces at 22117; the present invention utilizes diffuse reflection.
2. Efrati's photon source 22020 is exposed to photon flux within his target chamber, the present invention places photon sources outside of a treatment chamber.
3. Efrati's photon source 22020 essentially illuminates a hemisphere of volume perpendicular to the face of photon source 22020 and emits about half of its photons clockwise and half counterclockwise within a generally toroidal treatment volume; the present invention utilizes a narrow cone of photons directed in a singular or predominant clock direction.

Fig. PHO-22A and PHO-22B exhibit compact light-shielded trap 22300 with a trap frame 22110. Frame 22110 contains a reflective inner surface 22117, capable of substantially reflecting the light of light source 22020. Trap 22300 has inlet aperture 22111 and outlet aperture 22112. Trap 22300 is interconnected to light shield 22030 by connectors 22035.

Surfaces facing the interior of trap 22300 including surface 22031 can at least partially reflect light. An optional deflecting section 22032 of shield 22032 is aerodynamically designed to allow more linear air flow through the light trap. Additional inlet flange 22040 and outlet flange 22045 are added and shown in Fig. PHO-22B. These flanges allow easier and tighter connections to an HVAC system. Said light-shielded trap 22300 may be elongated as well and might also be shaped in other oval or cylindrical shapes. In this case, the light shield 22030 may be adjusted to the overall length of the trap. Alternatively, two separated light shields can cover an inner aperture and an outlet aperture.

The area outside of frame 22110 is in a passive shadow. This passive shadow is well suited for introduction of designated air 22011 through inlet flange 22040 to be polished from flow path 10134 illustrated in Fig. APS-01A and a supply of polished air 22112 from outlet flange 22045 into flow path 10135 illustrated in Fig. APS-01A.

Photonotron (Annular Cylindroid)—Independent claim

A system and method to increase photonic range of diffuse reflectors by confining photons on paths that maintain high angles of incidence and photonic cycling in a predominate clock direction within an annular cylindroid target chamber.
Photonotron (Annular Cylindroid)+predominate clock direction>90%, 99%, or 99.9%
Above+pre-polisher
Above+pre-polisher (HEPA)
Above+post polisher
Above+purge/dilution

### Photonotron (Cylindroid)—Independent Claim

A system and method to increase photonic range of diffuse reflectors by confining photons on paths predominated by high angles of incidence resulting in photonic cycling in a predominate clock direction within a cylindroid target chamber.
Photonotron (Cylindroid)+Lambertian cosine shadow @ orifice.
Above+pre-polisher
Above+pre-polisher (HEPA)
Above+post polisher
Above+purge/dilution

### Photonotron (Toroid)—Independent Claim

A system and method to increase photonic range of diffuse reflectors by confining photons on paths that maintain high angles of incidence and photonic cycling in a predominate clock direction within a toroidal target chamber.
Photonotron (Toroid)+predominate clock direction>90%, 99%, or 99.9%
Above+pre-polisher
Above+pre-polisher (HEPA)
Above+post polisher
Above+purge/dilution

### Attenuation Shadow—Independent Claim

A system and method to increase photonic range within a target chamber of photons emitted by at least one photon source by combining highly reflective materials and attenuation shadow(s) at least one LRS.
Above+pre-polisher
Above+pre-polisher (HEPA)
Above+post polisher
Above+purge/dilution

### Hybrid Target Chamber Reflection

Specular reflection systems and diffuse reflection systems may be combined to take advantage of their respective strengths and to overcome their respective weaknesses. Hybrid Composite Diffuse Reflection (HCDR) is one important example of such a combination and is described in its own section by that name. Another non-limiting example is a specular guardrail photon trap 2305 near orifice 2302 in an otherwise predominantly diffuse reflective target chamber. Orifice 2302 allows communication through target chamber wall 2300 between target chamber interior 2320 and target chamber exterior 2330. Orifices allow fluidic, photonic, or electronic communication between a target chamber and an exterior (Including but not limited to air inlets, air outlets, photon inlets, instruments, and instrument wiring). Considering again, Fig. PHO-06, but where an inner reflective surface is predominately diffusive, at least one photon is likely to strike locations adjacent to an orifice and might then be reflected into a low reflectivity orifice. Incident photon 2306 illustrated in Fig. PHO-23 would likely reflect into orifice 2302 absent guardrail photon trap 2305. Guardrail photon trap 2305 may include a step-like (i.e., rectilinear surface geometry) specular reflective structure 2305 counterclockwise from sawtooth 2301 and stepping counterclockwise into cylindroid wall 2304. A step dimension for at least one step 2305 is preferably larger than a wavelength of incident photon 2306. An individual step or a plurality of steps preferably covers a sufficient distance counterclockwise from sawtooth 2301 to minimize photons which might otherwise be directed into orifice 2302 in sawtooth 2301. A step-like structure employing right angles such as 2305 is well known in the art to reflect photons back in a direction, about parallel to an AOI from which they came. A step-like structure can be created through a manufacturing process such as, but not limited to, machining, 3D printing, molding, or by chemical processes such as crystals that intrinsically include nearly right angles. Specular guardrail photon traps extend counterclockwise from a counterclockwise sawtooth far enough such that a substantial portion of photons that would otherwise be funneled into orifice 2302 are deflected back into target chamber 0610 of Fig. PHO-06. Of course, a clockwise sawtooth is accompanied by a clockwise guardrail photon trap.

A second non-limiting example of hybrid system reflection, backstop photon trap (BPT) 2350, is illustrated in Fig. PHO-23. No matter the efficiency of passive shadowing and guardrail photon trap 2305, at least one stray photon 2351 is bound to enter the interior end of orifice 2302. Such stray photons would be of two types, (a) those with trajectories approximately parallel to orifice walls 2310 that do not strike orifice wall 2310 for an entire length of orifice 2302, and (b) those photons that do strike at least one wall 2310 of orifice 2302 as exemplified by ray 2351. For the latter case a preferred feature would be for an orifice to have specular reflective wall 2310 such that photon 2351 is encouraged to travel down a complete length of orifice 2302. A second implementation would use diffusive reflective wall 2310. As shown by Fig. PHO-03 a substantial portion of near glancing rays (i.e., high AOI) will undergo specular reflection. In either case (a) or case (b) when said photons reach the outside terminus of orifice 2302 at least one of four kinds of features may be encountered:
1) a photon source, and/or
2) a photon sensor, and/or
3) a non-photon sensor, and/or
4) a fluid inlet or outlet.

For feature (1) a direct path or a prismatic path is required, and its reflectivity is what it is and does not benefit from a BPT. For feature (2) there are two possible cases. If backstop 2350 has some transparency a photon detector may be located behind BPT mirror 2350. As a non-limiting example, a dielectric mirror on a quartz substrate might reflect 99.5% of incident ray 2352 and allow about 0.5% to pass. A photon detector behind such a mirror would measure about 1/200th of a photon flux incident upon BPT mirror 2350. In a second embodiment BPT 2350 is a mirror that allows zero photon transmission. Non-limiting examples include metal mirrors, where metals include aluminum, silver, and gold. If for any reason the detector is placed between orifice 2302 and BPT mirror 2350, detector reflectivity is what it is and no benefit of a BPT will be recognized. For features (3) and (4), BPT 2350 mirrored surface 2349 is generally perpendicular to orifice walls 2310, is located as close to the outside terminus of orifice 2302 without unduly restricting a function of said orifice (i.e., air flow or sensor function). In order of preference said mirror surface 2349 is:
1. A specular concave mirror which reflects incident photon 2352 back into orifice 2302 regardless of AOI. For example, incident ray 2352 strikes concave BPT 2350 and reflects ray 2354.
2. A specular photon reflector employing rectilinear surface geometry reflects incident photons on a parallel path to AOI (i.e., rectilinear surface geometry means all surface features are about) 90°. For example, incident ray 2352 strikes rectilinear reflector BPT 2350 and reflects as a new ray almost directly onto ray 2352 but in the opposite direction.
3. A specular flat mirror which reflects at least some incident photons back into the orifice. For example, incident ray 2352 strikes flat mirror BPT 2350 and reflects as ray 2353 and almost immediately strikes target chamber wall 2303. Ray 2353 is thus lost. Rays more parallel to walls 2310 and/or closer to the centerline of orifice 2302 are more likely to reflect into orifice 2302 and return to target chamber 2320.
4. A diffusive reflector which reflects at least some incident photons back in orifice 2302. Incident rays like ray 2352 will scatter in approximate Lambertian profile as illustrated in Fig. PHO-14. A non-zero fraction of ray 2352 will reflect into orifice 2302.

Orifice 2302 has a first end proximate target chamber 2320 and a second end distal to target chamber 2320. The performance of these 4 embodiments and particularly embodiments 3 and 4 improve if a second end of orifice 2302 was flared or chamfered (e.g., conically, parabolically, hyperbolically, catenary, etc.) to create a funneling effect to facilitate reentry of photons into orifice distal end of orifice 2302 which would otherwise strike outside an unchamfered distal end of orifice 2302.

### Guardrail Photon Trap (GPT)—Independent Claim

A system and method to increase photonic range by diverting at least some photons from striking a LRC in a diffuse reflective target chamber.
GPT+at least 1 specular reflective step
Above+pre-polisher
Above+pre-polisher (HEPA)
Above+post polisher
Above+purge/dilution

### Backstop Photon Trap (BPT)—Independent Claim

A system and method to improve system reflectivity by reflecting photons which have entered orifices back into the same orifices at about 180 degrees from their angle of incidence.
BPT+specular orifice inner walls
BPT+specular orifice inner walls+mirror to reflect some photons into orifice.
BPT+specular orifice inner walls+mirror to reflect some photons into orifice+concave mirror.
BPT+specular orifice inner walls+mirror to reflect some photons into orifice+rectilinear geometry mirror.
BPT+specular orifice inner walls+mirror to reflect some photons into orifice+rectilinear geometry mirror+chamfered orifice second end.
Above+pre-polisher
Above+pre-polisher (HEPA)
Above+post polisher
Above+purge/dilution

### Improved Photon Source Reflectivity

Any photon source such as a laser diode, an LED or a lamp is a poor photon reflector. Passively shadowing a photon source and providing guardrail photon traps can reduce the incident photon flux onto a photon source but cannot altogether eliminate incident photons. As system reflectivity is improved to the high 90% range, even a small drain of photons by a photon source may be problematic as illustrated by Fig. PHO-02. While prior art teaches an increase in the quantity of photon sources, the present invention reverses that teaching and seeks to minimize the number and size of photon sources. The following devices and methods seek to decrease surface area required for photon sources or improve the reflectivity of those surfaces that must remain:
1. Non-emitting surfaces and edges of any circuit board or other electrical connection on which a photon source is mounted or from which the photon source derives power may be covered in high reflectivity material. The choice of specular or diffusive reflectivity depends on a combination of design decisions in the target chamber as outlined in this document. All exposed surfaces of a photon source printed circuit board (PCB), other mounting structures, and including connections and wires, except emitting surfaces and associated emission cones that must be in an incident photon stream are covered in an enclosure, where an exterior surface of the enclosure is highly reflective.
2. Injection optics components can include reflective, refractive, TIR, or hybrid components, as well as imaging (e.g., lenses, parabola, ellipses, other conics) or non-imaging (e.g., edge-ray type including tailored-edge, non-edge, Compound parabolic concentrator (CPC), or other types of concentrators).
3. Heat must be transferred away from the photon source. The life of a photon source and the power which can be supplied to a photon source are limited by the temperature of a photon source. Air flow at typical room temperature is available in abundance as the present invention is designed to circulate air in a protectee-occupied volume. Methods to accomplish a high thermal flux include one or more of the following:
   a. A heat sink
   b. Air cooling (passive or active)
   c. A boiling/condensing fluid where boiling occurs at or near the photon source or an intimately connect heat sink thus cooling a photon source. The vaporized liquid (gaseous fluid) then condenses in a condenser situated in an ambient air flow. The fluid may be transported by gravity (i.e., boiled vapor rises and condensed fluid falls in a gravity field) or actively with a pump/compressor/fan. Active fluid transport allows the photon source to reach a lower temperature and operate in any orientation with respect to gravity or other gravity-like fields.
   d. A Peltier effect device (a.k.a. thermoelectric cooling).

### Minimized Photon Source—Independent Claim

A system and a method to improve target chamber reflectivity to minimize the surface area of poorly reflective photon sources.

### Reflective PCB Enclosure—Independent Claim

A system and method to improve target chamber reflectivity by covering non-emitting surfaces of a photon source with reflective surfaces.
Above+HEPA
Above+purge/dilution
Above+sorption

### Photonic ICE

Doubling the density of a compressible fluid including targets to be exposed to photons doubles photonic efficiency. At every temperature and pressure condition where a compressible fluid at least roughly follows the ideal gas law, there is a one-to-one relationship between density and photonic efficiency. At very high pressure and/or very low temperature, the one-to-one relationship falters, but a positive correlation continues until a compressible fluid liquifies. There are two independent ways to increase the density of a compressible fluid: (1) Increase pressure, and (2) decrease temperature. Either may be used alone or the two ways may be combined.

### Pressure

There are dozens of common compressor designs available on the market to increase the pressure of compressible gas at normal atmospheric pressure to values from 1.1- to 100-times a starting value. Any of these pumps provide a commensurate increase in photonic efficiency. There are certain applications (e.g., spacecraft, aircraft, submarines, scuba/scba) where compressed gas is required for breathing. Prior art compressors are appropriate for these cases.

These prior art pumps suffer a common shortcoming for applications where pressurized air is not required, namely there is no provision to recover at least a portion of the energy required to compress a gas. An ICE machine includes a provision to compress a next portion of gas at atmospheric pressure using energy stored in a previous portion of compressed gas.

Referring to Figs. ICE-01A and ICE-01B, a modified ICE machine follows these unique steps:
1. Designated air at about atmospheric pressure is drawn into at least one MVV, cylinder 0101 in Fig. ICE-01A.
2. Up to three power sources are applied to a MVV to compress gas in cylinder 0102 (Fig. ICE-01A) at nominally atmospheric pressure.
   a. Power provided by torque source 0142. E.g., electrical, combustion, steam, etc.
   b. Power harvested from expansion of previously compressed gas in cylinder 0104 (Fig. ICE-01A).
   c. Power added to compressed gas in cylinder 0104 (Fig. ICE-01A) by photon source 0134.
3. At least one of pre-compressed volume, compressed volume, and post-compressed volume is exposed to photons within an MVV. Photons may act directly on targets and/or may induce a photocatalytic reaction. Photon sources 0131, 0132, 0133, and 0134, together with respective orifices 0136, 0137, 0138, and 0139 provide photons to MVVs between heads 0106, 0107, 0108 and 0109, respective pistons 0111, 0112, 0113, and 0114, and respective cylinders 0101, 0102, 0103, and 0104. Photonic energy converts to head and may contribute to powering the ICE machine as indicated at step 2c.
4. A compressed volume in cylinder 0104 (Fig. ICE-01A) is allowed to return to atmospheric pressure by displacing piston 0114 and power so produced is harvested and returned at step 2b.
   a. Direct harvesting by mechanically linking at least two compression volumes such that one compression volume is undergoing compression while the other is undergoing expansion.
   b. Indirect harvesting by mechanically linking at least one compression volume to at least one mechanical energy storage device such as flywheel 0141. Other mechanical energy storage devices are possible and included in the scope of the present invention.
   c. Indirect harvesting by mechanically linking at least one compression volume to a dynamo to generate electricity. Said electricity may be stored to be used indirectly to power torque source 0142 of paragraph 2a, used directly to power torque source 0142 of paragraph 2a, or used for some other useful purpose (e.g., returned to the grid).
5. At the end of an expansion stroke, a decompressed and at least disinfected volume of polished air is pumped at about atmospheric pressure to flow path 10135 (Fig. APS-01A).
6. Return to step 1.

In one embodiment analogous to but not limited by an analogy to an internal combustion engine a compression volume is a piston-cylinder assembly. Step 1 is an intake stroke. Step 2 is a compression stroke. Step 3 occurs concurrently or at least a portion of Steps 1, 2, 4, and 5. Step 4 is an expansion stroke. Step 5 is an exhaust stroke. Preferably there are at least two compression volumes, and those two compression volumes are in opposing cycles of steps 1, 2, 4, and 5. That is, when a first compression volume is in steps 1, 2, 4, and 5, a second compression volume is in steps 4, 5, 1, and 2 respectively. Flywheels and valves like those used in four-stroke internal combustion engines smooth the operation and direct designated air flow and polished air flow. Preferably a compression volume includes at least one target chamber.

Compressed volumes may be created in structures analogous to piston engines, rotary engines, or turbines and with an integrated cycle or a split-cycle without departing from the spirit of the present invention. See section entitled Rotary ICE Machine.

In a second exemplary embodiment, a target chamber may be an oxidizer in a split-cycle ICE machine as illustrated in Fig. ICE-07. Optional internal components serpentine oxidizer 0751, three-way valve 0752, and three-way valve 0753 are omitted. In this embodiment oxidizer 0750 may be any practical volume and thus take advantage of longer mean photon flight paths. At least one photon source 0760 provides a stream of collimated or partially collimated photons into dual mechanism target chamber/oxidizer 0750 through optical orifice 0755. Optical orifice 0755 may include an optically clear cover to protect photon source 0760 from above ambient temperature and pressure within target chamber/oxidizer 0750.

The introduction of photons from photon source 0760 into target chamber/oxidizer 0750 creates a small departure from isothermal and isentropic operation. Photons convert to thermal energy when attenuated by reflection or when they interact with a target. In any case the supplied photonic energy increases the temperature of gas in oxidizer 0750. Photonic warming is a departure from ideal isothermal and isentropic operation but remains distinguished from engines that burn fuel to raise the temperature, not only by the source of the energy, but also in magnitude. Photonic warming is generally less than 1° C. and aways less than 5° C. This small departure from isothermal and isentropic operation does not have any material disadvantages. At least a portion of the photonic energy is recovered in the expansion step, which reduced the need for power from torque source 0790.

As a non-limiting example of potential improvement in photonic efficiency, a split-cycle ICE machine operates at a static compression ratio of 20 and an oxidizer diameter was about 20 times the diameter of a piston-cylinder arrangement, photonic efficiency would improve about 400-fold (i.e., the product of 20 and 20). Additionally, oxidizer 0750 shape may be optimized for photonic efficiency and reflective material choices for a static oxidizer may be broader than material choices for an MVV. These added benefits may generate an additional 2-times to about 10-times photonic efficiency. Finally, an ICE machine itself operates synergistically with irradiation on T1K utilizing IIL and IIO destroying T1K and oxidizable T3K with independent and complementary mechanisms.

### Temperature

In certain applications cryogenic air is stored to be released in protectee occupied areas. Some pathogens are known to survive indefinitely at cryogenic temperatures. As air, nitrogen, oxygen, helium and other gases are cooled, their density increases. When the absolute temperature of a gas is half of ambient, its density is twice as high. The doubling in density doubles photonic efficiency. Conventional cooling apparatus is utilized, and a target chamber is employed prior to gas liquefaction.

Where there is no desire to liquify gases, a gas can be cooled to increase its density to improve the photonic efficiency of a pathogen neutralizing device. To recover at least a portion of the energy required to cool a gas, a gas-to-gas heat exchanger may be provided to warm a gas after it has been treated in a target chamber and to partially cool untreated gas before it is delivered to a second stage of cooling powered by an external energy source and then to a target chamber.

### Density Boost—Independent Claim

A system and method to increase photonic efficiency of a target chamber by increasing density of a compressible fluid.
Increase density by increasing pressure.
Above+ICE to increase pressure, MVV=generic.
Above+ICE, MVV=piston-cylinder.
Above+ICE, MVV=cam guided rotary
Above+Isochoric split-cycle ICE
Above+Isochoric split-cycle ICE, MVV=piston-cylinder.
Above+Isochoric split-cycle ICE, MVV=cam guided rotary.
Above+shadow.
Above+shadow=clock.
Above+shadow=deflection.
Above+shadow=virtual.
Above+shadow=attenuation.
Above+shadow=Lambertian cosine.
Above+PRC.
Above+pre-polisher
Above+pre-polisher (HEPA)
Above+post polisher
Above+purge/dilution
Increase density by cooling.
Recover at least a portion of cooling energy with a heat exchanger.

### Hybrid Composite Diffuse Reflector (HCDR)

Reflective plastics such as native PTFE enjoy a property of reflection due to the duality of their makeup. They include a portion of their matrix that is amorphous and a portion that is crystalline. While the amorphous portion can fill any volume in any shape and are nominally transparent to light, crystalline portions form platelets and act as nano-scale mirrors. Manufactures of PTFE reflective materials add another variable to impact PTFE reflective behavior. Using sintering, expanding, and like processes, manufactures add occluded air spaces within a PTFE matrix creating bubble-like to dendritic micro-structure. Collectively these nanoscale and micro-scale structures yield generally Lambertian reflection for photons with low angles of incidence. Figure PHO-03 illustrates some relationships between AOI and a fraction of Lambertian and specular reflection for some diffuse reflective materials including PTFE. Reflective efficiency of such materials generally increases with thickness as some photons may be transmitted by numerous random reflections through the entirety of their thickness. As these materials can be costly and undue thickness adds weight or compromises other design considerations, layering, laminating, sputtering, or otherwise applying a metal such as aluminum, silver, or gold to an exterior surface of an inwardly reflective 3D surface is potentially useful and may increase a diffuse surface reflectivity as no photons are allowed to leak from an outer surface.

In Fig. PHO-25 hybrid composite diffuse reflector 2500 is composed of aluminum metal laminate 2510 and sintered PTFE laminate 2520. Six exemplary photons, 2501 through 2506 illustrate six exemplary but non-exhaustive behavior types. Photon 2506 strikes upper surface 2530 of PTFE laminate 2520 at a high AOI and undergoes specular reflection. Photon 2501 penetrates upper surface 2530 and reflects and refracts multiple times before it exits 2520 through surface 2530. Photon 2502 penetrates upper surface 2530 and reflects and refracts multiple times before it is absorbed by laminate 2520 and converted to heat. Photon 2503 penetrates upper surface 2530 and reflects and refracts multiple times including a single specular reflection from surface 2515 of aluminum laminate 2510 before it is subsequently absorbed by laminate 2520 and converted to heat. Photon 2504 penetrates upper surface 2530 and reflects and refracts multiple times including a single specular reflection from surface 2515 of aluminum laminate 2510 before it exits 2520 through surface 2530. Photon 2505 penetrates upper surface 2530 and reflects and refracts multiple times including two specular reflections from surface 2515 of aluminum laminate 2510 before it is absorbed by laminate 2520 and converted to heat. Photon 2504 would be lost if not for the presence of aluminum laminate 2510.

The benefit of this approach can be estimated with reference to Fig. PHO-24 which summarizes data from U.S. Pat. No. 10,800,672. Various thicknesses of a proprietary PTFE formulation are arrayed along X-axis 2410 from about 1 mm to about 10 mm. Reflectivity 2430, transmission 2440, and loss 2450 each expressed as a percentage of incident 285 nm wavelength UV light are plotted against lower Y-axis 2420 (0% to 15%) and upper Y-axis 2421 (about 75% to 100%).

For example, using a thickness of 3 mm of proprietary PTFE resin, approximately 5% of incident photons would transit a thickness to be reflected inward by an opaque reflector. Exemplary opaque reflectors include aluminum with about an 80-90% reflectance or a high-performance dielectric reflector at about 99% reflectance. Inwardly (i.e., toward a target chamber) reflected photons from aluminum surface 2515 must find their way back through PTFE laminate 2520 and about 5% would make the trip on a first interaction with opaque reflector 2510, but about 93% of those would be reflected outward (i.e., away from a target chamber), those 93% are reflected again inward by opaque reflector 2510. This process continues indefinitely and a gain from each iteration asymptotically approaches zero. With an 80% opaque reflector, performance increases from 93% to about 95.9%. With a 99% opaque reflector the performance increases from 93% to about 96.5%. In addition to the benefit of increased material and target chamber reflectivity a hybrid composite reflector utilizing an opaque reflector also precludes the escape of photons which might otherwise leak into an environment and create safety issues.

### Hybrid Composite Diffuse Reflector—Independent Claim

A system and method to increase component reflectance by improving diffuse reflectance of a polymeric material by including a specular reflector proximal and generally parallel to at least a portion of its exterior surface.
HCDR+specular reflector=opaque
HCDR+specular reflector=metal
HCDR+metal specular reflector=Al
HCDR+metal specular reflector=Al; PTFE is diffuse reflector
Above+pre-polisher
Above+pre-polisher (HEPA)
Above+post polisher
Above+purge/dilution

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms. For example, the use of the terms “a,” “an,” and “the” and similar referents in the context of describing the disclosed embodiments (especially in the context of the following claims) are to be construed to cover both the singular and the plural, unless otherwise indicated or clearly contradicted by context. Similarly, use of the term “or” is to be construed to mean “and/or” unless contradicted explicitly or by context. The terms “comprising,” “having,” “including,” and “containing” are to be construed as open-ended terms (i.e., meaning “including, but not limited to,”) unless otherwise noted.

The term “connected,” where unmodified and referring to physical connections, is to be construed as partly or wholly contained within, attached to, or joined together, even if there is something intervening. Recitation of ranges of values are merely intended to serve as a shorthand method of referring individually to each separate value falling within the range, unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal. The use of the phrase “based on,” unless otherwise explicitly stated or clear from context, means “based at least in part on” and is not limited to “based solely on.”

Conjunctive language, such as phrases of the form “at least one of A, B, and C,” or “at least one of A, B and C,” (i.e., the same phrase with or without the Oxford comma) unless specifically stated or otherwise clearly contradicted by context, is otherwise understood with the context as used in general to present that an item, term, etc., may be either A or B or C, any nonempty subset of the set of A and B and C, or any set not contradicted by context or otherwise excluded that contains at least one A, at least one B, or at least one C. For instance, in the illustrative example of a set that has three members, the conjunctive phrases “at least one of A, B, and C” and “at least one of A, B and C” refer to any of the following sets: {A}, {B}, {C}, {A, B}, {A, C}, {B, C}, {A, B, C}, and, if not contradicted explicitly or by context, any set that has {A}, {B}, and/or {C} as a subset (e.g., sets with multiple “A”). Thus, such conjunctive language is not generally intended to imply that certain embodiments require at least one of A, at least one of B and at least one of C each to be present. Similarly, phrases such as “at least one of A, B, or C” and “at least one of A, B or C” refer to the same as “at least one of A, B, and C” and “at least one of A, B and C” refer to any of the following sets: {A}, {B}, {C}, {A, B}, {A, C}, {B, C}, {A, B, C}, unless differing meaning is explicitly stated or clear from context. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

It should be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” or “one or more” to introduce claim recitations. However, the use of such phrases do not imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Accordingly, the invention is not limited except as by the appended claims.

The use of any examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

What is claimed is:

1. An air polisher photonic target chamber for cleaning an airflow having entrained targets with photons from a photon source, comprising:
an input aperture positioned to receive the airflow into the target chamber;
an output aperture positioned to exhaust the airflow from the target chamber, with an internal target chamber airflow extending between the input and output apertures;
a target chamber interior having a target chamber reflectance (R_{TC}), at least one discrete unshadowed portion (m) with an area (Au,m) having a homogenous material reflectance (R_{u,m}), and at least one discrete shadowed portion (n) with an area (As,n) having a homogenous reflectance (Rs,n) and a homogenous shadow (Sn), where the areas Au,m and As,n together comprise the total interior area of the target chamber; and
a chamber structure causing the chamber to have a relative improvement of greater than 1% in the variance from ideal that is greater than 1%,
where R_{TC}={[ΣA_{u,m}+Σ(A_{s,n}·(1−Sₙ))]·Rᵤ+Σ(A_{s,n}·Rₙ·Sₙ)}/(ΣA_{u,m}+ΣA_{s,n}),
and
where Rᵤ=Σ(A_{u,m}·R_{u,m})/ΣA_{u,m}
and
where summations include all discrete areas from 1 to m and from 1 to n, as appropriate to respective subscripts m and n.

2. The air polisher photonic target chamber of claim 1, wherein the chamber is structured to cause the chamber to have a relative improvement that is greater than 5%.

3. The air polisher photonic target chamber of claim 1, wherein the chamber is structured to cause the chamber to have a relative improvement that is greater than 50%.

4. The air polisher photonic target chamber of claim 1, wherein the chamber is structured to cause the chamber to have a relative improvement that is greater than 75%.

5. An air polisher system usable with an air source providing an airflow having entrained targets, comprising:
a photon source operable to generate photons;
a pre-polisher positioned to receive the airflow from the air source, the pre-polisher being structured to at least partially remove from the airflow one or more of radioactive aerosols, biological substances, vapor phase chemicals, aerosolized chemicals, carbon dioxide, and particulate matter from the airflow and produce a partially cleaned output airflow; and
a photonic target chamber, having:
an input aperture positioned to receive the partially cleaned output airflow from the pre-polisher into the target chamber to be further cleaned with photons from the photon source;
an output aperture positioned to exhaust the airflow from the target chamber, with an internal target chamber airflow extending between the input and output apertures;
a target chamber interior having a target chamber reflectance (R_{TC}), at least one discrete unshadowed portion (m) with an area (A_{u,m}) having a homogenous material reflectance (R_{u,m}), and at least one discrete shadowed portion (n) with an area (A_{s,n}) having a homogenous reflectance (R_{s,n}) and a homogenous shadow (Sn), where the areas A_{u,m} and A_{s,n} together comprise the total interior area of the target chamber; and
a chamber structure causing the chamber to have a relative improvement that is greater than 1%,
where R_{TC}={[ΣA_{u,m}+Σ(A_{s,n}·(1−Sₙ))]·Rᵤ+Σ(A_{s,n}·Rₙ·Sₙ)}/(ΣA_{u,m}+ΣA_{s,n}),
and
where Rᵤ=Σ(A_{u,m}·R_{u,m})/ΣA_{u,m}
and
where summations include all discrete areas from 1 to m and from 1 to n, as appropriate to respective subscripts m and n.

6. The air polisher system of claim 5, further including a post-polisher positioned to receive the airflow from the output aperture of the photonic target chamber, the post-polisher being structured to at least partially remove from the received airflow one or more of radioactive aerosols, biological substances, vapor phase chemicals, aerosolized chemicals, and carbon dioxide from the received airflow and produce a further cleaned output airflow.

7. The air polisher system of claim 5, wherein the photonic target chamber is structured to cause the chamber to have a relative improvement that is greater than 5%.

8. The air polisher system of claim 7, further including a post-polisher positioned to receive the airflow from the output aperture of the photonic target chamber, the post-polisher being structured to at least partially remove from the received airflow one or more of radioactive aerosols, biological substances, vapor phase chemicals, aerosolized chemicals, and carbon dioxide from the received airflow and produce a further cleaned output airflow.

9. The air polisher system of claim 5, wherein the photonic target chamber is structured to cause the chamber to have a relative improvement that is greater than 50%.

10. The air polisher system of claim 9, further including a post-polisher positioned to receive the airflow from the output aperture of the photonic target chamber, the post-polisher being structured to at least partially remove from the received airflow one or more of radioactive aerosols, biological substances, vapor phase chemicals, aerosolized chemicals, and carbon dioxide from the received airflow and produce a further cleaned output airflow.

11. The air polisher system of claim 5, wherein the photonic target chamber is structured to cause the chamber to have a relative improvement that is greater than 75%.

12. The air polisher system of claim 11, further including a post-polisher positioned to receive the airflow from the output aperture of the photonic target chamber, the post-polisher being structured to at least partially remove from the received airflow one or more of radioactive aerosols, biological substances, vapor phase chemicals, aerosolized chemicals, and carbon dioxide from the received airflow and produce a further cleaned output airflow.

13. The air polisher system of claim 5, wherein the pre-polisher is a HEPA filter.

14. An air polisher system, comprising:
an at least partially enclosed space containing air having at least one type of entrained target at a first concentration;
a source of air having the at least one type of entrained target at a second concentration less than the first concentration, the source of air being in controlled fluid communication with the enclosed space to supply air with the second concentration to the enclosed space;
a valve positioned to control the flow of supply air from the source with the second concentration to the enclosed space to reduce the concentration of the at least one type of entrained target in the air of the enclosed space to a third concentration less than the first concentration;
a photon source operable to generate photons; and
a photonic target chamber, having:
an input aperture in fluid communication with the enclosed space to receive into the target chamber an airflow from the enclosed space with the third concentration of the at least one type of entrained target to be cleaned with photons from the photon source;
an output aperture positioned to exhaust the airflow from the target chamber, with an internal target chamber airflow extending between the input and output apertures;
a target chamber interior having a target chamber reflectance (R_{TC}), at least one discrete unshadowed portion (m) with an area (Au,m) having a homogenous material reflectance (Ru,m), and at least one discrete shadowed portion (n) with an area (As,n) having a homogenous reflectance (Rs,n) and a homogenous shadow (Sn), where the areas Au,m and As,n together comprise the total interior area of the target chamber; and
a chamber structure causing the chamber to have a relative improvement that is greater than 1%,
where R_{TC}={[ΣA_{u,m}+Σ(A_{s,n}·(1−Sₙ))]·Rᵤ+Σ(A_{s,n}·Rₙ·Sₙ)}/(ΣA_{u,m}+ΣA_{s,n}),
and
where Rᵤ=Σ(A_{u,m}·R_{u,m})/ΣA_{u,m}
and
where summations include all discrete areas from 1 to m and from 1 to n, as appropriate to respective subscripts m and n.

15. The air polisher system of claim 14, wherein the photonic target chamber is structured to cause the chamber to have a relative improvement that is greater than 5%.

16. The air polisher system of claim 14, wherein the photonic target chamber is structured to cause the chamber to have a relative improvement that is greater than 50%.

17. The air polisher system of claim 14, wherein the photonic target chamber is structured to cause the chamber to have a relative improvement that is greater than 75%.

18. An air polisher photonic target chamber for cleaning an airflow having entrained targets with photons from a photon source, comprising:
an input aperture positioned to receive the airflow into the target chamber;
an output aperture positioned to exhaust the airflow from the target chamber, with an internal target chamber airflow extending between the input and output apertures; and
a target chamber interior having at least one movable interior chamber reflective wall surface selectively movable within the target chamber interior between a first position and a second position, in the first position the movable wall surface allows a first rate of internal target chamber airflow, and in the second position the movable wall surface allows a second rate of internal chamber airflow larger than the first rate of internal chamber airflow, such that when the movable wall surface is in the first position the air within the target chamber interior gets an enhanced photon exposure than when the movable wall surface is in the second position.

19. The air polisher photonic target chamber of claim 18, wherein when in the first position the movable wall surface at least partially covers at least one of the input aperture and output aperture.

20. The air polisher photonic chamber of claim 18, wherein at least one of the inlet aperture and outlet aperture is a virtual wall.

21. The air polisher photonic target chamber of claim 18, wherein when in the first position the movable wall surface at least partially covers a portion of the target chamber interior that has a lower reflectance than the reflectance of the reflective wall surface of the target chamber interior.

22. The air polisher photonic target chamber of claim 21, wherein at least a portion of the target chamber interior that has the lower reflectance is a virtual wall.

23. The air polisher photonic target chamber of claim 18, wherein at least a portion of the of the movable wall surface has a reflective wall surface portion with a first reflectance and at least a portion of the target chamber interior has a second reflectance lower than the first reflectance, and when in the first position the reflective wall surface with the first reflectance at least partially covers the portion of the target chamber with the second reflectance.

24. An air polisher system usable for cleaning air having entrained targets with photons, comprising:
at least first and second photonic target chambers, each of the first and second target chambers having:
an input aperture positioned to receive the airflow into the target chamber;
an output aperture positioned to exhaust the airflow from the target chamber, with an internal target chamber airflow extending between the input and output apertures; and
a target chamber interior having a movable interior chamber reflective wall surface selectively movable within the target chamber interior between a first position and a second position, in the first position the movable wall surface allows a first rate of internal target chamber airflow, and in the second position the movable wall surface allows a second rate of internal chamber airflow larger than the first rate of internal chamber airflow, such that when the movable wall surface is in the first position the air within the target chamber interior gets an enhanced photon exposure than when the movable wall surface is in the second position; and
at least one actuator operable to move the movable wall surfaces of the first and second target chambers between the first and second positions thereof, and to sequentially initiate the movements of the movable wall surfaces of the first and second target chambers between the first and second positions thereof.

25. The air polisher system of claim 24, further including an air polisher system output aperture in fluid communication with the output aperture of the first target chamber and the output aperture of the second target chamber to exhaust air from the air polisher system.

26. The air polisher system of claim 24, further including:
at least one photon source operable to generate pulses of photons for illumination of the target chamber interior of the first and second target chambers when the movable wall surface of the first target chamber is in the first position thereof and the movable wall surface of the second target chamber is in the first position thereof.

27. The air polisher system of claim 24, wherein the at least one actuator is configured such that during a single cycle the air volume flowing through the first target chamber is less than the total interior volume of the first target chamber and the air volume flowing through the second target chamber is less than the total interior volume of the second target chamber.

28. The air polisher system of claim 27, wherein the at least one actuator includes a rotatable cam wheel operable in a single full rotation to move each of the movable wall surfaces of the first and second target chambers between the first and second positions thereof, the angular velocity of the cam wheel being selected such that during the single rotation of the cam wheel the air volume flowing through the first target chamber is less than the total interior volume of the first target chamber and the air volume flowing through the second target chamber is less than the total interior volume of the second target chamber.

29. An air polisher system usable with an air source providing an airflow having entrained targets, comprising:
a photon source operable to generate photons;
a pre-polisher positioned to receive the airflow from the air source, the pre-polisher being structured to at least partially remove from the airflow one or more of radioactive aerosols, biological substances, vapor phase chemicals, aerosolized chemicals, carbon dioxide, and particulate matter from the airflow and produce a partially cleaned output airflow; and
a photonic target chamber, having:
an input aperture positioned to receive the partially cleaned output airflow from the pre-polisher into the target chamber to be further cleaned with photons from the photon source;
an output aperture positioned to exhaust the airflow from the target chamber, with an internal target chamber airflow extending between the input and output apertures; and
a target chamber interior having a movable interior chamber reflective wall surface selectively movable within the target chamber interior between a first position and a second position, in the first position the movable wall surface allows a first rate of internal target chamber airflow, and in the second position the movable wall surface allows a second rate of internal chamber airflow larger than the first rate of internal chamber airflow, such that when the movable wall surface is in the first position the air within the target chamber interior gets an enhanced photon exposure than when the movable wall surface is in the second position.

30. The air polisher system of claim 29, wherein the movable wall surface changes the geometry of a virtual wall as the movable wall surface moves.

31. The air polisher system of claim 29, wherein the pre-polisher is a HEPA filter.

32. The air polisher system of claim 29, further including a post-polisher positioned to receive the airflow from the output aperture of the photonic target chamber, the post-polisher being structured to at least partially remove from the received airflow one or more of radioactive aerosols, biological substances, vapor phase chemicals, aerosolized chemicals, and carbon dioxide from the received airflow and produce a further cleaned output airflow.

33. An air polisher system, comprising:
an at least partially enclosed space containing air having at least one type of entrained target at a first concentration;
a source of air having the at least one type of entrained target at a second concentration less than the first concentration, the source of air being in controlled fluid communication with the enclosed space to supply air with the second concentration to the enclosed space;
a valve positioned to control the flow of supply air from the source with the second concentration to the enclosed space to reduce the concentration of the at least one type of entrained target in the air of the enclosed space to a third concentration less than the first concentration;
a photon source operable to generate photons; and
a photonic target chamber, having:
an input aperture in fluid communication with the enclosed space to receive into the target chamber an airflow from the enclosed space with the third concentration of the at least one type of entrained target to be cleaned with photons from the photon source;
an output aperture positioned to exhaust the airflow from the target chamber, with an internal target chamber airflow extending between the input and output apertures; and
a target chamber interior having a movable interior chamber reflective wall surface selectively movable within the target chamber interior between a first position and a second position, in the first position the movable wall surface allows a first rate of internal target chamber airflow, and in the second position the movable wall surface allows a second rate of internal chamber airflow larger than the first rate of internal chamber airflow, such that when the movable wall surface is in the first position the air within the target chamber interior gets an enhanced photon exposure than when the movable wall surface is in the second position.

34. The air polisher system of claim 33, wherein the movable wall surface changes the geometry of a virtual wall as the moveable wall surface moves.
